(19) United States
(12) Patent Application Publication
(10) Pub. No.: US 2014/0171357 A1
(43) Pub. Date: Jun. 19, 2014

(54) VANCOMYCIN DERIVATIVES

(71) Applicant: Seachald Pharmaceuticals, Inc., Durham, NC (US)

(72) Inventors: Balasingam Radhakrishnan, Chapel Hill, NC (US); Ronald George Sherrill, Cary, NC (US); Kenneth Duke James, JR., Mebane, NC (US)

(73) Assignee: Seachaid Pharmaceuticals, Inc., Durham, NC (US)

(21) Appl. No.: 14/006,868

(22) PCT Filed: Mar. 23, 2012
(86) PCT No.: PCT/US2012/030323

§ 371 Date: Mar. 4, 2014

Related U.S. Application Data
(60) Provisional application No. 61/467,082, filed on Mar. 24, 2011.

(51) Int. Cl. C07K 9/00

(52) U.S. Cl. CPC C07K 9/008
USPC 514/1.3; 530/322; 514/2.7; 514/2.6; 514/2.9

(57) ABSTRACT

The invention features vancomycin class compounds modified to be suitable for oral delivery or to possess increased antimicrobial potency, formulations for the oral administration of vancomycin class compounds, and synthetic methods for making vancomycin class compounds.

### BACKGROUND OF THE INVENTION

This invention relates to the field of treatment of bacterial infections.

Vancomycin is a naturally occurring glycopeptide antibiotic that is active against Gram-positive bacteria. It is produced extraribosomally and consists of two pyranose residues and seven amino acids, the latter of which are significantly cross-linked to maintain the structural integrity of the molecule. Though broadly active against Gram-positive bacteria, its best known use is against strains of methicillin resistant Staphylococcus aureus (MRSA). Despite having been discovered over fifty years ago, vancomycin remains a very important therapeutic in the antibacterial armamentarium. Because vancomycin is poorly absorbed after oral administration, it is currently dosed intravenously to treat systemic infections.

New compounds and formulation technologies are needed to improve on the existing therapies. The present invention addresses these problems and features compounds suitable for oral delivery, formulations for the oral administration of vancomycin class compounds, and synthetic methods for making vancomycin class compounds.

### SUMMARY OF THE INVENTION

The invention features vancomycin class compounds modified to be suitable for oral delivery or to possess increased antimicrobial potency, formulations for the oral administration of vancomycin class compounds, and synthetic methods for making vancomycin class compounds.

A compound of formula (I), or a salt or prodrug thereof:

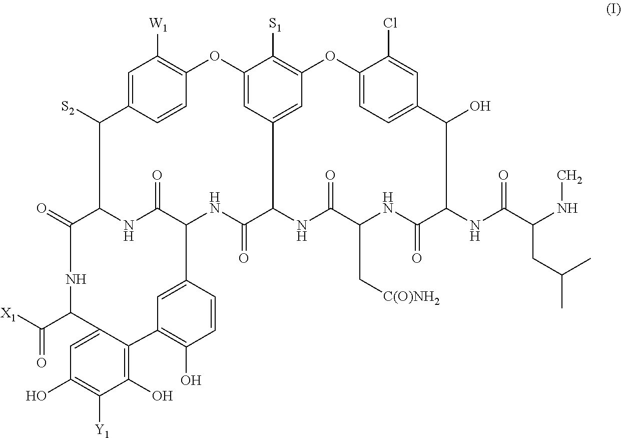

In formula (I), W₁ is H or Cl; X₁ is selected from N(R^{A})(CH₂CH₂O)ₐCH₂CH₂Z₁, OH, NH₂, NHR^{A1}, NR^{A1}R^{A2}, and OR^{A1}; Y₁ is selected from CH₂N(R^{B})(CH₂CH₂O)_{b}CH₂CH₂Z₂, H, CH₂NH₂, CH₂NHCOR^{B1}, CH₂NHCONHR^{B1}, CH₂NHCONR^{B1}R^{B2}, CH₂NHC(O)OR^{B1}, CH₂NHR^{B1}, CH₂NR^{B1}R^{B2}; CH₂NHSO₂R^{B1}, CH₂NHSO₂NHR^{B1}, CH₂NHSO₂NR^{B1}R^{B2}, and CH₂NHCH₂PO(OH)₂; S₁ is a saccharide group selected from:

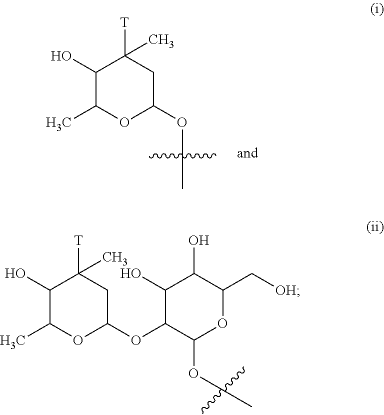

T is selected from —NH₂, —NH(CH₂)_{c}NHR^{T1}, —NHCO(CH₂)_{c}NHR^{T1}, —NHR^{T1}, —NH(CH₂)_{c}R^{T1}, and —NHCH₂—(C₆H₄)_{c}—O—R^{T1}; S₂ is OH or

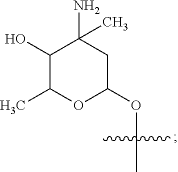 [SMILES: *OC1CC(C)(N)C(O)C(C)O1]

a is an integer from 1 to 20 (e.g., 1, 2, 3, 4, 5, 6, 7, or 8, from 1 to 4, from 2 to 5, from 2 to 12, from 3 to 6, or from 3 to 20); b is an integer from 1 to 20 (e.g., 1, 2, 3, 4, 5, 6, 7, or 8, from 1 to 4, from 2 to 5, from 2 to 12, from 3 to 6, or from 3 to 20); c is an integer from 1 to 3 (e.g., 1, 2, or 3); each of R^{A} and R^{B} is, independently, selected from H and C₁₋₄ alkyl; each of R^{A1} and R^{A2} is, independently, selected from C₁₋₁₂ alkyl,
C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; each of R^{B1} and R^{B2} is, independently, selected from C₁₋₁₂ alkyl, C₂₋₁₂ alkenyl, C₂₋₁₂ alkynyl, C₆₋₁₂ aryl, alkaryl, C₃₋₁₀ alkheterocyclyl, and C₁₋₁₂ heteroalkyl; R^{T1} is selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; each of Z₁ and Z₂ is, independently, selected from NH₂, NHR^{C1}, NR^{C1}R^{C2}; and NR^{C1}R^{C2}R^{C3}; each of R^{C1}, R^{C2}, and R^{C3} is, independently, selected from C₁₋₄ alkyl, C₂₋₄ alkenyl, and C₂₋₄ alkynyl, provided that either X₁ is N(R^{A})(CH₂CH₂O)ₐCH₂CH₂Z₁ or Y₁ is CH₂N(R^{B})(CH₂CH₂O)_{b}CH₂CH₂Z₂. In certain embodiments, T is selected from —NH₂, —NH(CH₂)₉CH₃, —NHCH₂CH₂NH(CH₂)₉CH₃, p-(p-chlorophenyl)benzyl-NH—, 4-phenylbenzyl-NH—, and 4-[(3,4-dichlorophenyl)methoxy]benzyl-NH—. In still other embodiments, at least one of Z₁ and Z₂ is a quaternary amine. In particular embodiments, each of Z₁ and Z₂ is, independently, selected from —NH₂, —N(CH₃)₂, and —N(CH₃)₃.

The compounds of formula (I) can further be described by formula (II), or a salt or prodrug thereof:

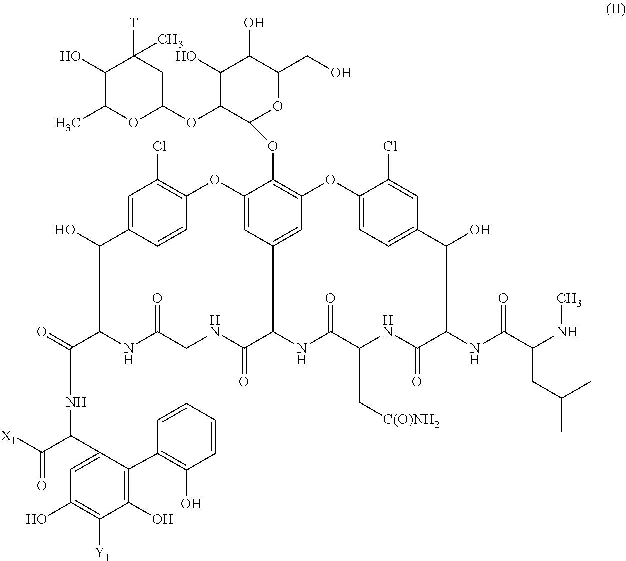

In formula (II), X₁, Y₁, and T are as defined in formula (I). For example, in certain embodiments of the compound of formula (II) T is —NH₂, X₁ is OH, NH₂, NHR^{A1}, NR_{A1}R^{A2}; and OR^{A1}; Y₁ is CH₂N(R^{B})(CH₂CH₂O)_{b}CH₂CH₂Z₂; each of R^{A1} and R^{A2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{B} is H or C₁₋₄ alkyl; b is an integer from 1 to 10 (e.g., 1, 2, 3, 4, 5, 6, 7, or 8, from 1 to 4, from 2 to 5, from 2 to 10, or from 3 to 10); Z₂ is NH₂, NHR^{C1}, NR^{C1}R^{C2}, or NR^{C1}R^{C2}R^{C3}; and each of R^{C1}, R^{C2}, and R^{C3} is, independently, selected from C₁₋₄ alkyl, C₂₋₄ alkenyl, and C₂₋₄ alkynyl, or a salt or prodrug thereof. In some embodiments of the compound of formula (II), T is —NH(CH₂)₉CH₃, X₁ is OH, NH₂, NHR^{A1}, NR^{A1}R^{A2}, and OR^{A1}; Y₁ is CH₂N(R^{B})(CH₂CH₂O)_{b}CH₂CH₂Z₂; each of R^{A1} and R^{A2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{B} is H or C₁₋₄ alkyl; b is an integer from 1 to 10 (e.g., 1, 2, 3, 4, 5, 6, 7, or 8, from 1 to 4, from 2 to 5, from 2 to 10, or from 3 to 10); Z₂ is NH₂, NHR^{C1}, NR^{C1}R^{C2}, or NR^{C1}R^{C2}R^{C3}; and each of R^{C1}, R^{C2}, and R^{C3} is, independently, selected from C₁₋₄ alkyl, C₂₋₄ alkenyl, and C₂₋₄ alkynyl, or a salt or prodrug thereof. In still other embodiments of the compound of formula (II), T is —NHCH₂CH₂NH(CH₂)₉CH₃, X₁ is OH, NH₂, NHR^{A1}, NR^{A1}R^{A2}, and OR^{A1}; Y₁ is CH₂N(R^{B})(CH₂CH₂O)_{b}CH₂CH₂Z₂; each of R^{A1} and R^{A2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{B} is H or C₁₋₄ alkyl; b is an integer from 1 to 10 (e.g., 1, 2, 3, 4, 5, 6, 7, or 8, from 1 to 4, from 2 to 5, from 2 to 10, or from 3 to 10); Z₂ is NH₂, NHR^{C1}, NR^{C1}R^{C2}; or NR^{C1}R^{C2}R^{C3}; and each of R^{C1}, R^{C2}, and R^{C3} is, independently, selected from C₁₋₄ alkyl, C₂₋₄ alkenyl, and C₂₋₄ alkynyl, or a salt or prodrug thereof. In particular embodiments of the compound of formula (II), T is p-(p-chlorophenyl)benzyl-NH—, X₁ is OH, NH₂, NHR^{A1}, NR^{A1}R^{A2}, and OR^{A1}; Y₁ is CH₂N(R^{B})(CH₂CH₂O)_{b}CH₂CH₂Z₂; each of R^{A1} and R^{A2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{B} is H or C₁₋₄ alkyl; b is an integer from 1 to 10 (e.g., 1, 2, 3, 4, 5, 6, 7, or 8, from 1 to 4, from 2 to 5, from 2 to 10, or from 3 to 10); Z₂ is NH₂, NHR^{C1}, NR^{C1}R^{C2}, or NR^{C1}R^{C2}R^{C3}; and each of R^{C1}, R^{C2}, and R^{C3} is, independently, selected from C₁₋₄ alkyl, C₂₋₄ alkenyl, and C₂₋₄ alkynyl, or a salt or prodrug thereof. In certain embodiments of the compound of formula (II), T is 4-phenylbenzyl-NH—, X₁ is OH, NH₂, NHR^{A1}, NR^{A1}R^{A2}, and OR^{A1}; Y₁ is CH₂N(R^{B})(CH₂CH₂O)_{b}CH₂CH₂Z₂; each of R^{A1} and R^{A2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{B} is H or C₁₋₄ alkyl; b is an integer from 1 to 10 (e.g., 1, 2, 3, 4, 5, 6, 7, or 8, from 1 to 4, from 2 to 5, from 2 to 10, or from 3 to 10); Z₂ is NH₂, NHR^{C1}, NR^{C1}R^{C2}, or NR^{C1}R^{C2}R^{C3}; and each of R^{C1}, R^{C2}, and R^{C3} is, independently, selected from C₁₋₄ alkyl, C₂₋₄ alkenyl, and C₂₋₄ alkynyl, or a salt or prodrug thereof. In still other embodiments of the compound of formula (II), T is 4-[(3,4-dichlorophenyl)methoxy]benzyl-NH—, X₁ is OH, NH₂, NHR^{A1}, NR^{A1}R^{A2}, and OR^{A1}; Y₁ is CH₂N(R^{B})(CH₂CH₂O)_{b}CH₂CH₂Z₂; each of R^{A1} and R^{A2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{B} is H or C₁₋₄ alkyl; b is an integer from 1 to 10 (e.g., 1, 2, 3, 4, 5, 6, 7, or 8, from 1 to 4, from 2 to 5, from 2 to 10, or from 3 to 10); Z₂ is NH₂, NHR^{C1}, NR^{C1}R^{C2}, or NR^{C1}R^{C2}R^{C3}; and each of R^{C1}, R^{C2}, and R^{C3} is, independently, selected from C₁₋₄ alkyl, C₂₋₄ alkenyl, and C₂₋₄ alkynyl, or a salt or prodrug thereof. In particular embodiments of the compound of formula (II), T is —NH₂, X₁ is N(R^{A})(CH₂CH₂O)ₐCH₂CH₂Z₁, Y₁ is selected from H, CH₂NH₂, CH₂NHCOR^{B1}, CH₂NHCONHR^{B1}, CH₂NHCONR^{B1}R^{B2}, CH₂NHC(O)OR^{B1}, CH₂NHR^{B1}, CH₂NR^{B1}R^{B2}; CH₂NHSO₂R^{B1}, CH₂NHSO₂NHR^{B1}, CH₂NHSO₂NR^{B1}R^{B2}, and CH₂NHCH₂PO(OH)₂; each of R^{B1} and R^{B2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{A} is H or C₁₋₄ alkyl; a is an integer from 1 to 10 (e.g., 1, 2, 3, 4, 5, 6, 7, or 8, from 1 to 4, from 2 to 5, from 2 to 10, or from 3 to 10); Z₂ is NH₂, NHR^{C1}, NR^{C1}R^{C2}; or NR^{C1}R^{C2}R^{C3}; and each of R^{C1}, R^{C2} and R^{C3} is, independently, selected from C₁₋₄ alkyl, C₂₋₄ alkenyl, and C₂₋₄ alkynyl, or a salt or prodrug thereof. In still other embodiments of the compound of formula (II), T is —NH(CH₂)₉CH₃, X₁ is N(R^{A})(CH₂CH₂O)ₐCH₂CH₂Z₁, Y₁ is selected from H, CH₂NH₂, CH₂NHCOR^{B1}, CH₂NHCONHR^{B1}, CH₂NHCONR^{B1}R^{B2}, CH₂NHC(O)OR^{B1}, CH₂NHR^{B1}, CH₂NR^{B1}R^{B2}; CH₂NHSO₂R^{B1}, CH₂NHSO₂NHR^{B1}, CH₂NHSO₂NR^{B1}R^{B2}, and CH₂NHCH₂PO(OH)₂; each of R^{B1} and R^{B2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{A} is H or C₁₋₄ alkyl; a is an integer from 1 to 10 (e.g., 1, 2, 3, 4, 5, 6, 7, or 8, from 1 to 4, from 2 to 5, from 2 to 10, or from 3 to 10); Z₂ is NH₂, NHR^{C1}, NR^{C1}R^{C2}, or NR^{C1}R^{C2}R^{C3}; and each of R^{C1}, R^{C2} and R^{C3} is, independently, selected from C₁₋₄ alkyl, C₂₋₄ alkenyl, and C₂₋₄ alkynyl, or a salt or prodrug thereof. In certain embodiments of the compound of formula (II), T is —NHCH₂CH₂NH(CH₂)₉CH₃, X₁ is N(R^{A})(CH₂CH₂O)ₐCH₂CH₂Z₁, Y₁ is selected from H, CH₂NH₂, CH₂NHCOR^{B1}, CH₂NHCONHR^{B1}, CH₂NHCONR^{B1}R^{B2}, CH₂NHC(O)OR^{B1}, CH₂NHR^{B1}, CH₂NR^{B1}R^{B2}; CH₂NHSO₂R^{B1}, CH₂NHSO₂NHR^{B1}, CH₂NHSO₂NR^{B1}R^{B2}, and CH₂NHCH₂PO(OH)₂; each of R^{B1} and R^{B2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{A} is H or C₁₋₄ alkyl; a is an integer from 1 to 10 (e.g., 1, 2, 3, 4, 5, 6, 7, or 8, from 1 to 4, from 2 to 5, from 2 to 10, or from 3 to 10); Z₂ is NH₂, NHR^{C1}, NR^{C1}R^{C2}, or NR^{C1}R^{C2}R^{C3}; and each of R^{C1}, R^{C2} and R^{C3} is, independently, selected from C₁ alkyl, C₂₋₄ alkenyl, and C₂₋₄ alkynyl, or a salt or prodrug thereof. In particular embodiments of the compound of formula (II), T is p-(p-chlorophenyl)benzyl-NH—, X₁ is N(R^{A})(CH₂CH₂O)ₐCH₂CH₂Z₁, Y₁ is selected from H, CH₂NH₂, CH₂NHCOR^{B1}, CH₂NHCONHR^{B1}, CH₂NHCONR^{B1}R^{B2}, CH₂NHC(O)OR^{B1}, CH₂NHR^{B1}, CH₂NR^{B1}R^{B2}; CH₂NHSO₂R^{B1}, CH₂NHSO₂NHR^{B1}, CH₂NHSO₂NR^{B1}R^{B2}, and CH₂NHCH₂PO(OH)₂; each of R^{B1} and R^{B2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{A} is H or C₁₋₄ alkyl; a is an integer from 1 to 10 (e.g., 1, 2, 3, 4, 5, 6, 7, or 8, from 1 to 4, from 2 to 5, from 2 to 10, or from 3 to 10); Z₂ is NH₂, NHR^{C1}, NR^{C1}R^{C2}, or NR^{C1}R^{C2}R^{C3}; and each of R^{C1}, R^{C2} and R^{C3} is, independently, selected from C₁₋₄ alkyl, C₂₋₄ alkenyl, and C₂₋₄ alkynyl, or a salt or prodrug thereof. In still other embodiments of the compound of formula (II), T is 4-phenylbenzyl-NH—, X₁ is N(R^{A})(CH₂CH₂O)ₐCH₂CH₂Z₁, Y₁ is selected from H, CH₂NH₂, CH₂NHCOR^{B1}, CH₂NHCONHR^{B1}, CH₂NHCONR^{B1}R^{B2}, CH₂NHC(O)OR^{B1}, CH₂NHR^{B1}, CH₂NR^{B1}R^{B2}; CH₂NHSO₂R^{B1}, CH₂NHSO₂NHR^{B1}, CH₂NHSO₂NR^{B1}R^{B2}, and CH₂NHCH₂PO(OH)₂; each of R^{B1} and R^{B2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{A} is H or C₁₋₄ alkyl; a is an integer from 1 to 10 (e.g., 1, 2, 3, 4, 5, 6, 7, or 8, from 1 to 4, from 2 to 5, from 2 to 10, or from 3 to 10); Z₂ is NH₂, NHR^{C1}, NR^{C1}R^{C2}, or NR^{C1}R^{C2}R^{C3}; and each of R^{C1}, R^{C2} and R^{C3} is, independently, selected from C₁₋₄ alkyl, C₂₋₄ alkenyl, and C₂₋₄ alkynyl, or a salt or prodrug thereof. In certain embodiments of the compound of formula (II), T is 4-[(3,4-dichlorophenyl)methoxy]benzyl-NH—, X₁ is N(R^{A})(CH₂CH₂O)ₐCH₂CH₂Z₁, Y₁ is selected from H, CH₂NH₂, CH₂NHCOR^{B1}, CH₂NHCONHR^{B1}, CH₂NHCONR^{B1}R^{B2}, CH₂NHC(O)OR^{B1}, CH₂NHR^{B1}, CH₂NR^{B1}R^{B2}; CH₂NHSO₂R^{B1}, CH₂NHSO₂NHR^{B1}, CH₂NHSO₂NR^{B1}R^{B2}, and CH₂NHCH₂PO(OH)₂; each of R^{B1} and R^{B2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{A} is H or C₁₋₄ alkyl; a is an integer from 1 to 10 (e.g., 1, 2, 3, 4, 5, 6, 7, or 8, from 1 to 4, from 2 to 5, from 2 to 10, or from 3 to 10); Z₂ is NH₂, NHR^{C1}, NR^{C1}R^{C2}, or NR^{C1}R^{C2}R^{C3}; and each of R^{C1}, R^{C2} and R^{C3} is, independently, selected from C₁₋₄ alkyl, C₂₋₄ alkenyl, and C₂₋₄ alkynyl, or a salt or prodrug thereof.

In particular embodiments of the compound of formula (II), T is —NH₂, X₁ is N(R^{A})(CH₂CH₂O)ₐCH₂CH₂Z₁, Y₁ is CH₂N(R^{B})(CH₂CH₂O)_{b}CH₂CH₂Z₂, each of R^{A} and R^{B} is, independently, selected from H and C₁₋₄ alkyl, a is an integer from 1 to 10 (e.g., 1, 2, 3, 4, 5, 6, 7, or 8, from 1 to 4, from 2 to 5, from 2 to 10, or from 3 to 10), b is an integer from 1 to 10 (e.g., 1, 2, 3, 4, 5, 6, 7, or 8, from 1 to 4, from 2 to 5, from 2 to 10, or from 3 to 10), each of Z₁ and Z₂ is, independently, selected from NH₂, NHR^{C1}, NR^{C1}R^{C2}, and NR^{C1}R^{C2}R^{C3} and each of R^{C1}, R^{C2}, and R^{C3} is, independently, selected from C₁₋₄ alkyl, C₂₋₄ alkenyl, and C₂₋₄ alkynyl, or a salt or prodrug thereof. In certain embodiments of the compound of formula (II), T is —NH(CH₂)₉CH₃, X₁ is N(R^{A})(CH₂CH₂O)ₐCH₂CH₂Z₁, Y₁ is CH₂N(R^{B})(CH₂CH₂O)_{b}CH₂CH₂Z₂, each of R^{A} and R^{B} is, independently, selected from H and C₁₋₄ alkyl, a is an integer from 1 to 10 (e.g., 1, 2, 3, 4, 5, 6, 7, or 8, from 1 to 4, from 2 to 5, from 2 to 10, or from 3 to 10), b is an integer from 1 to 10 (e.g., 1, 2, 3, 4, 5, 6, 7, or 8, from 1 to 4, from 2 to 5, from 2 to 10, or from 3 to 10), each of Z₁ and Z₂ is, independently, selected from NH₂, NHR^{C1}, NR^{C1}R^{C2}, and NR^{C1}R^{C2}R^{C3}, and each of R^{C1}, R^{C2}, and R^{C3} is, independently, selected from C₁₋₄ alkyl, C₂₋₄ alkenyl, and C₂₋₄ alkynyl, or a salt or prodrug thereof. In still other embodiments of the compound of formula (II), T is —NHCH₂CH₂NH(CH₂)₉CH₃, X₁ is N(R^{A})(CH₂CH₂O)ₐCH₂CH₂Z₁, Y₁ is CH₂N(R^{B})(CH₂CH₂O)_{b}CH₂CH₂Z₂, each of R^{A} and R^{B} is, independently, selected from H and C₁₋₄ alkyl, a is an integer from 1 to 10 (e.g., 1, 2, 3, 4, 5, 6, 7, or 8, from 1 to 4, from 2 to 5, from 2 to 10, or from 3 to 10), b is an integer from 1 to 10 (e.g., 1, 2, 3, 4, 5, 6, 7, or 8, from 1 to 4, from 2 to 5, from 2 to 10, or from 3 to 10), each of Z₁ and Z₂ is, independently, selected from NH₂, NHR^{C1}, NR^{C1}R^{C2}, and NR^{C1}R^{C2}R^{C3}, and each of R^{C1}, R^{C2}, and R^{C3} is, independently, selected from C₁₋₄ alkyl, C₂₋₄ alkenyl, and C₂₋₄ alkynyl, or a salt or prodrug thereof. In particular embodiments of the compound of formula (II), T is p-(p-chlorophenyl)benzyl-NH—, X₁ is N(R^{A})(CH₂CH₂O)ₐCH₂CH₂Z₁, Y₁ is CH₂N(R^{B})(CH₂CH₂O)_{b}CH₂CH₂Z₂, each of R^{A} and R^{B} is, independently, selected from H and C₁₋₄ alkyl, a is an integer from 1 to 10 (e.g., 1, 2, 3, 4, 5, 6, 7, or 8, from 1 to 4, from 2 to 5, from 2 to 10, or from 3 to 10), b is an integer from 1 to 10 (e.g., 1, 2, 3, 4, 5, 6, 7, or 8, from 1 to 4, from 2 to 5, from 2 to 10, or from 3 to 10), each of Z₁ and Z₂ is, independently, selected from NH₂, NHR^{C1}, NR^{C1}R^{C2}, and NR^{C1}R^{C2}R^{C3}, and each of R^{C1}, R^{C2}, and R^{C3} is, independently, selected from C₁₋₄ alkyl, C₂₋₄ alkenyl, and C₂₋₄ alkynyl, or a salt or prodrug thereof. In certain embodiments of the compound of formula (II), T is 4-phenylbenzyl-NH—, X₁ is N(R^{A})(CH₂CH₂O)ₐCH₂CH₂Z₁, Y₁ is CH₂N(R^{B})(CH₂CH₂O)_{b}CH₂CH₂Z₂, each of R^{A} and R^{B} is, independently, selected from H and C₁₋₄ alkyl, a is an integer from 1 to 10 (e.g., 1, 2, 3, 4, 5, 6, 7, or 8, from 1 to 4, from 2 to 5, from 2 to 10, or from 3 to 10), b is an integer from 1 to 10 (e.g., 1, 2, 3, 4, 5, 6, 7, or 8, from 1 to 4, from 2 to 5, from 2 to 10, or from 3 to 10), each of Z₁ and Z₂ is, independently, selected from NH₂, NHR^{C1}, NR^{C1}R^{C2}, and NR^{C1}R^{C2}R^{C3} and each of R^{C1}, R^{C2}, and R^{C3} is, independently, selected from C₁₋₄ alkyl, C₂₋₄ alkenyl, and C₂₋₄ alkynyl, or a salt or prodrug thereof. In still other embodiments of the compound of formula (II), T is 4-[(3,4-dichlorophenyl)methoxy]benzyl-NH—, X₁ is N(R^{A})(CH₂CH₂O)ₐCH₂CH₂Z₁, Y₁ is CH₂N(R^{B})(CH₂CH₂O)_{b}CH₂CH₂Z₂, each of R^{A} and R^{B} is, independently, selected from H and C₁₋₄ alkyl, a is an integer from 1 to 10 (e.g., 1, 2, 3, 4, 5, 6, 7, or 8, from 1 to 4, from 2 to 5, from 2 to 10, or from 3 to 10), b is an integer from 1 to 10 (e.g., 1, 2, 3, 4, 5, 6, 7, or 8, from 1 to 4, from 2 to 5, from 2 to 10, or from 3 to 10), each of Z₁ and Z₂ is, independently, selected from NH₂, NHR^{C1}, NR^{C1}R^{C2}, and NR^{C1}R^{C2}R^{C3} and each of R^{C1}, R^{C2}, and R^{C3} is, independently, selected from C₁₋₄ alkyl, C₂₋₄ alkenyl, and C₂₋₄ alkynyl, or a salt or prodrug thereof.

The compounds of formula (I) can further be described by formula (VI), or a salt or prodrug thereof:

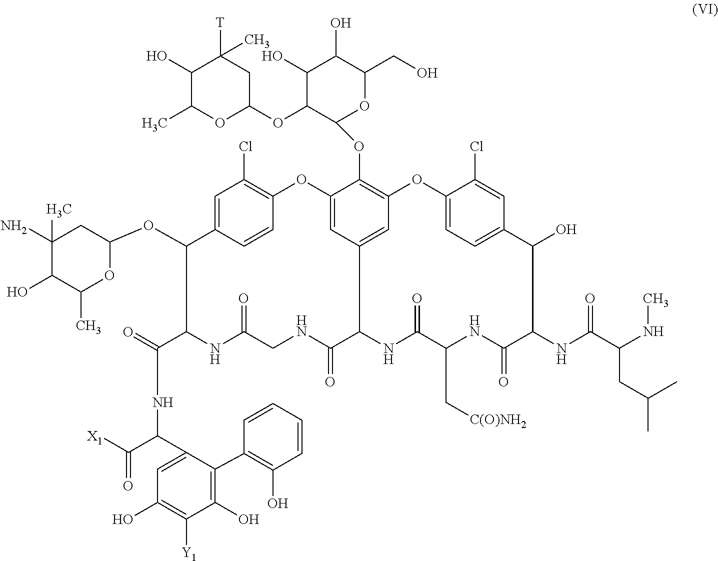

In formula (VI), X₁, Y₁, and T are as defined in formula (I). In particular embodiments, T is selected from —NH₂, —NH(CH₂)₉CH₃, —NHCH₂CH₂NH(CH₂)₉CH₃, p-(p-chlorophenyl)benzyl-NH—, 4-phenylbenzyl-NH—, and 4-[(3,4-dichlorophenyl)methoxy]benzyl-NH—. For example, in certain embodiments of the compound of formula (VI) T is —NH₂, X₁ is OH, NH₂, NHR^{A1}, NR^{A1}R^{A2}, and OR^{A1}; Y₁ is CH₂N(R^{B})(CH₂CH₂O)_{b}CH₂CH₂Z₂; each of R^{A1} and R^{A2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{B} is H or C₁₋₄ alkyl; b is an integer from 1 to 10 (e.g., 1, 2, 3, 4, 5, 6, 7, or 8, from 1 to 4, from 2 to 5, from 2 to 10, or from 3 to 10); Z₂ is NH₂, NHR^{C1}, NR^{C1}R^{C2}, or NR^{C1}R^{C2}R^{C3}, and each of R^{C1}, R^{C2}, and R^{C3} is, independently, selected from C₁₋₄ alkyl, C₂₋₄ alkenyl, and C₂₋₄ alkynyl, or a salt or prodrug thereof. In particular embodiments of the compound of formula (VI), T is —NH₂, X₁ is N(R^{A})(CH₂CH₂O)ₐCH₂CH₂Z₁, Y₁ is selected from H, CH₂NH₂, CH₂NHCOR^{B1}, CH₂NHCONHR^{B1}, CH₂NHCONR^{B1}R^{B2}, CH₂NHC(O)OR^{B1}, CH₂NHR^{B1}, CH₂NR^{B1}R^{B2}; CH₂NHSO₂R^{B1}, CH₂NHSO₂NHR^{B1}, CH₂NHSO₂NR^{B1}R^{B2}, and CH₂NHCH₂PO(OH)₂; each of R^{B1} and R^{B2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{A} is H or C₁₋₄ alkyl; a is an integer from 1 to 10 (e.g., 1, 2, 3, 4, 5, 6, 7, or 8, from 1 to 4, from 2 to 5, from 2 to 10, or from 3 to 10); Z₂ is NH₂, NHR^{C1}, NR^{C1}R^{C2}; or NR^{C1}R^{C2}R^{C3}; and each of R^{C1}, R^{C2} and R^{C3} is, independently, selected from C₁₋₄ alkyl, C₂₋₄ alkenyl, and C₂₋₄ alkynyl, or a salt or prodrug thereof. In some embodiments of the compound of formula (VI), T is —NH₂, X₁ is N(R^{A})(CH₂CH₂O)ₐCH₂CH₂Z₁, Y₁ is CH₂N(R^{B})(CH₂CH₂O)_{b}CH₂CH₂Z₂, each of R^{A} and R^{B} is, independently, selected from H and C₁₋₄ alkyl, a is an integer from 1 to 10 (e.g., 1, 2, 3, 4, 5, 6, 7, or 8, from 1 to 4, from 2 to 5, from 2 to 10, or from 3 to 10), b is an integer from 1 to 10 (e.g., 1, 2, 3, 4, 5, 6, 7, or 8, from 1 to 4, from 2 to 5, from 2 to 10, or from 3 to 10), each of Z₁ and Z₂ is, independently, selected from NH₂, NHR^{C1}, NR^{C1}R^{C2}, and NR^{C1}R^{C2}R^{C3}, and each of R^{C1}, R^{C2}, and R^{C3} is, independently, selected from C₁₋₄ alkyl, C₂₋₄ alkenyl, and C₂₋₄ alkynyl, or a salt or prodrug thereof. In certain embodiments of the compound of formula (VI), T is p-(p-chlorophenyl)benzyl-NH—, X₁ is OH, NH₂, NHR^{A1}, NR^{A1}R^{A2}; and OR^{A1}; Y₁ is CH₂N(R^{B})(CH₂CH₂O)_{b}CH₂CH₂Z₂; each of R^{A1} and R^{A2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{B} is H or C₁₋₄ alkyl; b is an integer from 1 to 10 (e.g., 1, 2, 3, 4, 5, 6, 7, or 8, from 1 to 4, from 2 to 5, from 2 to 10, or from 3 to 10); Z₂ is NH₂, NHR^{C1}, NR^{C1}R^{C2}, NR^{C1}R^{C2}R^{C3}; and each of R^{C1}, R^{C2}, and R^{C3} is, independently, selected from C₁₋₄ alkyl, C₂₋₄ alkenyl, and C₂₋₄ alkynyl, or a salt or prodrug thereof. In particular embodiments of the compound of formula (VI), T is p-(p-chlorophenyl)benzyl-NH—, X₁ is N(R^{A})(CH₂CH₂O)ₐCH₂CH₂Z₁, Y₁ is selected from H, CH₂NH₂, CH₂NHCOR^{B1}, CH₂NHCONHR^{B1}, CH₂NHCONR^{B1}R^{B2}; CH₂NHC(O)OR^{B1}, CH₂NHR^{B1}, CH₂NR^{B1}R^{B2}; CH₂NHSO₂R^{B1}, CH₂NHSO₂NHR^{B1}, CH₂NHSO₂NR^{B1}R^{B2}; and CH₂NHCH₂PO(OH)₂; each of R^{B1} and R^{B2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{A} is H or C₁₋₄ alkyl; a is an integer from 1 to 10 (e.g., 1, 2, 3, 4, 5, 6, 7, or 8, from 1 to 4, from 2 to 5, from 2 to 10, or from 3 to 10); Z₂ is NH₂, NHR^{C1}, NR^{C1}R^{C2}, or NR^{C1}R^{C2}R^{C3}; and each of R^{C1}, R^{C2} and R^{C3} is, independently, selected from C₁₋₄ alkyl, C₂₋₄ alkenyl, and C₂₋₄ alkynyl, or a salt or prodrug thereof. In some embodiments of the compound of formula (VI), T is p-(p-chlorophenyl)benzyl-NH—, X₁ is N(R^{A})(CH₂CH₂O)ₐCH₂CH₂Z₁, Y₁ is CH₂N(R^{B})(CH₂CH₂O)_{b}CH₂CH₂Z₂, each of R^{A} and R^{B} is, independently, selected from H and C₁₋₄ alkyl, a is an integer from 1 to 10 (e.g., 1, 2, 3, 4, 5, 6, 7, or 8, from 1 to 4, from 2 to 5, from 2 to 10, or from 3 to 10), b is an integer from 1 to 10 (e.g., 1, 2, 3, 4, 5, 6, 7, or 8, from 1 to 4, from 2 to 5, from 2 to 10, or from 3 to 10), each of Z₁ and Z₂ is, independently, selected from NH₂, NHR^{C1}, NR^{C1}R^{C2}, and NR^{C1}R^{C2}R^{C3}, and each of R^{C1}, R^{C2}, and R^{C3} is, independently, selected from C₁₋₄ alkyl, C₂₋₄ alkenyl, and C₂₋₄ alkynyl, or a salt or prodrug thereof.

The compounds of formula (I) can further be described by formula (VII), or a salt or prodrug thereof:

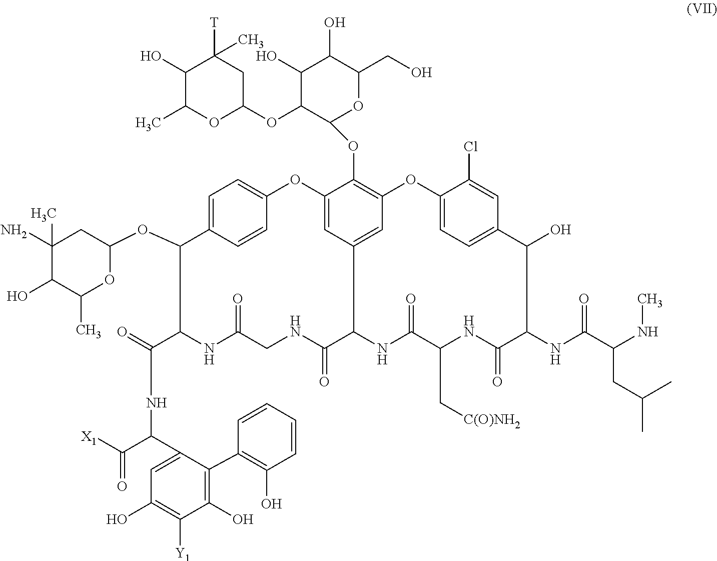

In formula (VII), X₁, Y₁, and T are as defined in formula (I). In particular embodiments, T is selected from —NH₂, —NH(CH₂)₉CH₃, —NHCH₂CH₂NH(CH₂)₉CH₃, p-(p-chlorophenyl)benzyl-NH—, 4-phenylbenzyl-NH—, and 4-[(3,4-dichlorophenyl)methoxy]benzyl-NH—. For example, in certain embodiments of the compound of formula (VII) T is —NH₂, X₁ is OH, NH₂, NHR^{A1}, NR^{A1}R^{A2}, and OR^{A1}; Y₁ is CH₂N(R^{B})(CH₂CH₂O)_{b}CH₂CH₂Z₂; each of R^{A1} and R^{A2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{B} is H or C₁₋₄ alkyl; b is an integer from 1 to 10 (e.g., 1, 2, 3, 4, 5, 6, 7, or 8, from 1 to 4, from 2 to 5, from 2 to 10, or from 3 to 10); Z₂ is NH₂, NHR^{C1}, NR^{C1}R^{C2}, or NR^{C1}R^{C2}R^{C3}; and each of R^{C1}, R^{C2}, and R^{C3} is, independently, selected from C₁₋₄ alkyl, C₂₋₄ alkenyl, and C₂₋₄ alkynyl, or a salt or prodrug thereof. In particular embodiments of the compound of formula (VII), T is —NH₂, X₁ is N(R^{A})(CH₂CH₂O)ₐCH₂CH₂Z₁, Y₁ is selected from H, CH₂NH₂, CH₂NHCOR^{B1}, CH₂NHCONHR^{B1}, CH₂NHCONR^{B1}R^{B2}, CH₂NHC(O)OR^{B1}, CH₂NHR^{B1}, CH₂NR^{B1}R^{B2}; CH₂NHSO₂R^{B1}, CH₂NHSO₂NHR^{B1}, CH₂NHSO₂NR^{B1}R^{B2}, and CH₂NHCH₂PO(OH)₂; each of R^{B1} and R^{B2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{A} is H or C₁₋₄ alkyl; a is an integer from 1 to 10 (e.g., 1, 2, 3, 4, 5, 6, 7, or 8, from 1 to 4, from 2 to 5, from 2 to 10, or from 3 to 10); Z₂ is NH₂, NHR^{C1}, NR^{C1}R^{C2}, or NR^{C1}R^{C2}R^{C3}; and each of R^{C1}, R^{C2} and R^{C3} is, independently, selected from C₁₋₄ alkyl, C₂₋₄ alkenyl, and C₂₋₄ alkynyl, or a salt or prodrug thereof. In particular embodiments of the compound of formula (VII), T is —NH₂, X₁ is N(R^{A})(CH₂CH₂O)ₐCH₂CH₂Z₁, Y₁ is CH₂N(R^{B})(CH₂CH₂O)_{b}CH₂CH₂Z₂, each of R^{A} and R^{B} is, independently, selected from H and C₁₋₄ alkyl, a is an integer from 1 to 10 (e.g., 1, 2, 3, 4, 5, 6, 7, or 8, from 1 to 4, from 2 to 5, from 2 to 10, or from 3 to 10), b is an integer from 1 to 10 (e.g., 1, 2, 3, 4, 5, 6, 7, or 8, from 1 to 4, from 2 to 5, from 2 to 10, or from 3 to 10), each of Z₁ and Z₂ is, independently, selected from NH₂, NHR^{C1}, NR^{C1}R^{C2}, and NR^{C1}R^{C2}R^{C3}, and each of R^{C1}, R^{C2}, and R^{C3} is, independently, selected from C₁₋₄ alkyl, C₂₋₄ alkenyl, and C₂₋₄ alkynyl, or a salt or prodrug thereof.

The compounds of formula (I) can further be described by formula (VIII), or a salt or prodrug thereof:

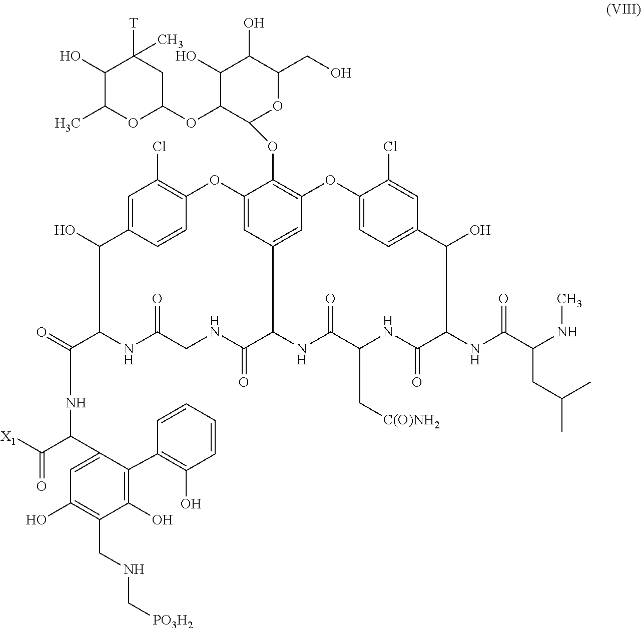

In formula (VIII), X₁ and T are as defined in formula (I). In particular embodiments, T is selected from —NH₂, —NH(CH₂)₉CH₃, —NHCH₂CH₂NH(CH₂)₉CH₃, p-(p-chlorophenyl)benzyl-NH—, 4-phenylbenzyl-NH—, and 4-[(3,4-dichlorophenyl)methoxy]benzyl-NH—. In certain embodiments of the compound of formula (VIII), T is —NHCH₂CH₂NH(CH₂)₉CH₃, X₁ is N(R^{A})(CH₂CH₂O)ₐCH₂CH₂Z₁, R^{A} is H or C₁₋₄ alkyl; a is an integer from 1 to 10 (e.g., 1, 2, 3, 4, 5, 6, 7, or 8, from 1 to 4, from 2 to 5, from 2 to 10, or from 3 to 10); Z₂ is NH₂, NHR^{C1}, NR^{C1}R^{C2}, NR^{C1}R^{C2}R^{C3}; and each of R^{C1}, R^{C2}, and R^{C3} is, independently, selected from C₁₋₄ alkyl, C₂₋₄ alkenyl, and C₂₋₄ alkynyl, or a salt or prodrug thereof.

In a related aspect, the invention features a pharmaceutical composition including a compound of any of formulas (I), (II), (IIIa)-(IIIf), (IVa)-(IVf), (Va)-(Vf), (VI), (VII), or (VIII), or a salt or prodrug thereof, and a pharmaceutically acceptable excipient.

The invention features a pharmaceutical composition in oral dosage form including a vancomycin class compound, or a salt or prodrug thereof, and an additive selected from sugar esters, alkyl saccharides, acyl carnitines, glycerides, chitosan and derivatives thereof, amido fatty acids, fatty acids and salts or esters thereof, polyethylene glycol alkyl ethers, poly-D-lysine, N-acetyl-L-cystine, and combinations thereof, wherein the additive is present in an amount sufficient to increase the oral bioavailability of the vancomycin class compound. The pharmaceutical composition can be an oral dosage form that is a liquid dosage form or a solid dosage form, optionally in a unit dosage form. In particular embodiments, the pharmaceutical composition includes from 15% to 90% (w/w) of the additive (e.g., from 15% to 35%, 25% to 50%, 40% to 60%, 55% to 75%, or from 70% to 90% (w/w) additive) and from 5% to 30% (w/w) of the vancomycin class compound (e.g., from 5% to 10%, 7.5% to 15%, 10% to 20%, 15% to 25%, or from 20% to 30% (w/w) vancomycin class compound). In certain embodiments, the oral dosage form includes a (w/w) ratio of the vancomycin class compound to the additive of from 1:0.5 to 1:16 (e.g., a (w/w) ratio of from 1:1 to 1:16, 1:1.5 to 1:10, 1:2 to 1:12, 1:1.5 to 1:5, or 1:3 to 1:10). In particular embodiments, the additive is the sugar ester sucrose monolaurate or sucrose monocaprate. In still other embodiments, the additive is a alkyl saccharide selected from octyl maltoside, decyl maltoside, dodecyl maltoside, tetradecyl maltoside, dodecyl glucoside, and decyl glucoside. The additive can be an acyl carnitine selected from palmitoyl carnitine, decanoyl carnitine, and dodecanoyl carnitine. In certain embodiment, the additive is a glyceride formed from a mixture of fatty acids or salts or esters thereof, a mixture of monoglycerides, and/or a mixture of diglycerides, and/or a mixture of triglycerides. In particular embodiments, the additive is a chitosan, or a derivative thereof, selected from chitosan, trimethylchitosan, and chitosan-4-thio-butylamidine. The additive can be the amido fatty acid sodium N-[8-(2-hydroxybenzoyl)amino]caprylate. The additive can be the fatty acid salt sodium caprylate, sodium caprate, or sodium laurate. In certain other embodiments, the additive is a polyethylene glycol alkyl ether selected from Laureth 9, Laureth 12, and Laureth 20. In still other embodiments, the additive is poly-D-lysine or N-acetyl-L-cystine. The vancomycin class compound can be a compound of formula (I), or a vancomycin class compound selected from vancomycin, teicoplanin, dalbavancin, telavancin, oritavancin, eremomycin, and chloroeremomycin. In particular embodiments, the additive is a combination of the components described herein (e.g., acyl carnitines with chitosan or derivatives thereof, such as palmitoyl carnitine with trimethyl chitosan; poly-D-Lysine with chitosan or derivatives thereof; amido fatty acids with glycerides; sugar ester with alkyl saccharides; polyethylene glycol alky ethers with N-acetyl L-cystine; or polyethylene glycol alky ethers with sugar esters or alkyl saccharides).

The invention features a method of treating a bacterial infection in a subject by administering a compound of any of formulas (I), (II), (IIIa)-(IIIf), (IVa)-(IVf), (Va)-(Vf), (VI), (VII), or (VIII), or a salt or prodrug thereof, or a pharmaceutical composition in oral dosage form including a vancomycin class compound to the subject a compound in an amount sufficient to treat the infection. The bacterial infection to be treated can be selected from community-acquired pneumonia, upper and lower respiratory tract infection, skin and soft tissue infection, bone and joint infection, hospital-acquired lung infection, acute bacterial otitis media, bacterial pneumonia, complicated infection, noncomplicated infection, pyelonephritis, intra-abdominal infection, deep-seated abcess, bacterial sepsis, central nervous system infection, bacteremia, wound infection, peritonitis, meningitis, infections after burn, urogenital tract infection, gastro-intestinal tract infection, pelvic inflammatory disease, endocarditis, intravascular infection, complicated skin and skin structure infection, complicated intra-abdominal infection, hospital acquired pneumonia, ventilator associated pneumonia, pseudomembranous colitis, enterocolitis, infections associated with prosthetics or dialysis, and any other infection described herein. The compound can also be administered for prophylaxis against an infection associated with a surgical procedure or implantation of a prosthetic device (e.g., preoperative antimicrobial prophylaxis). In particular embodiments, the compound is administered orally. In still other embodiments, the compound is administered intravenously. The compounds can be used to treat infections caused by, for example, Staphylococcus spp; Streptococcus spp; Enterococcus spp; Clostridium spp; Bacillus spp; Staphylococcus aureus, including methicillin-susceptible (MSSA), methicillin-resistant (MRSA), vancomycin-intermediate (VISA), heterogeneous VISA (hVISA), and vancomycin-resistant (VRSA) strains; Staphylococcus epidermidis, including methicillin susceptible and resistant strains; Enterococcus faecium, including VanA-type (VRE) and VanB-type (VRE) resistant strains; Enterococcus faecalis, including VanA-type (VRE) and VanB-type (VRE) resistant strains; Enterococcus casseliflavus and Enterococcus gallinarum, including vanC-carrying strains; Streptococcus pneumoniae, including multi-drug resistant strains; Streptococcus pyogenes and Streptococcus agalactiae, including 3-hemolytic strains; and Bacillus anthracis, or any other bacterial species described herein.

The invention also features a method of killing a bacterial cell by contacting the cell with a compound of any of formulas (I), (II), (IIIa)-(IIIf), (IVa)-(IVf), (Va)-(Vf), (VI), (VII), or (VIII), or a salt or prodrug thereof, in an amount sufficient to kill the bacterial cell. The bacterial cell can be selected from Staphylococcus spp; Streptococcus spp; Enterococcus spp; Clostridium spp; Bacillus spp; Staphylococcus aureus, including methicillin-susceptible (MSSA), methicillin-resistant (MRSA), vancomycin-intermediate (VISA), heterogeneous VISA (hVISA), and vancomycin-resistant (VRSA) strains; Staphylococcus epidermidis, including methicillin susceptible and resistant strains; Enterococcus faecium, including VanA-type (VRE) and VanB-type (VRE) resistant strains; Enterococcus faecalis, including VanA-type (VRE) and VanB-type (VRE) resistant strains; Enterococcus casseliflavus and Enterococcus gallinarum, including vanC-carrying strains; Streptococcus pneumoniae, including multi-drug resistant strains; Streptococcus pyogenes and Streptococcus agalactiae, including β-hemolytic strains; and Bacillus anthracis, or any other bacterial species described herein.

The invention further features a method of synthesizing the acid addition salt of a compound of formula (X):

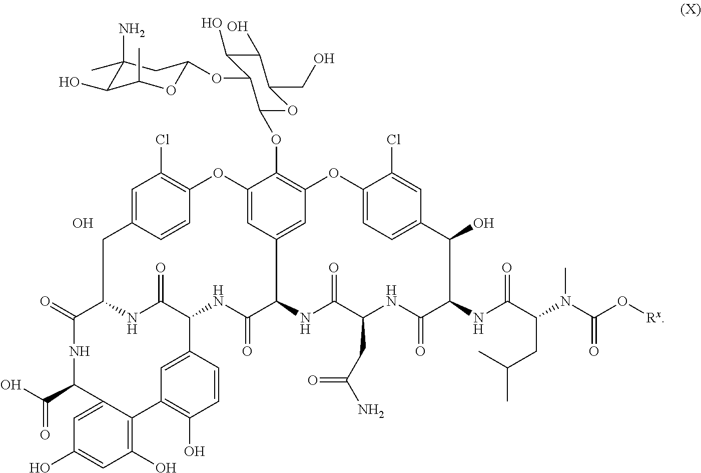

In formula (X), R^{X} is selected from C₁₋₁₂ alkyl, C₂₋₁₂ alkenyl, C₂₋₁₂ alkynyl, and C₇₋₁₆ alkaryl. The method includes the step of reacting the mono acid addition salt of vancomycin with a dicarbonate in an organic solvent to form an acid addition salt of a compound of formula (X), the dicarbonate having the formula
R^{X}—OC(O)—O—C(O)O—R^{X} (wherein R^{X} is as defined above in formula (X)), and wherein the ratio of acid to vancomycin is from about 0.85:1 to 1.15:1 (e.g., from 0.90:1 to 1.10:1, from 0.95:1 to 1.05:1, from 0.97:1 to 1.03:1, or from 0.98:1 to 1.02:1). In certain embodiments, the method further includes the steps of (i) dissolving vancomycin, or an acid addition salt thereof, in an organic solvent and (ii) adjusting the pH of the solution with base or acid to produce a ratio of acid to vancomycin of from about 0.85:1 to 1.15:1 (e.g., from 0.90:1 to 1.10:1, from 0.95:1 to 1.05:1, from 0.97:1 to 1.03:1, or from 0.98:1 to 1.02:1) prior to reaction with the dicarbonate. In particular embodiments of the method, the acid addition salt of vancomycin is selected from vancomycin hydrochloride, vancomycin hydrobromide, vancomycin hydroiodide, vancomycin sulfate, vancomycin phosphate and vancomycin methansulfonate. In certain embodiments of the method, the dicarbonate is selected from di-tert-butyl dicarbonate, dibenzyl dicarbonate, and diallyl dicarbonate.

In a related aspect, the invention features a method of synthesizing a vancomycin class compound by (i) synthesizing the carbamate-protected vancomycin of formula (X), or a salt thereof, (ii) alkylating the amine bearing saccharide group of the carbamate-protected vancomycin, coupling an amine to the C-terminal carboxylate of the carbamate-protected vancomycin, and/or adding an aminomethyl substituent the resorcinol ring of the carbamate-protected vancomycin via a Mannich reaction, and (iii) removing the carbamate protecting group to produce a vancomycin class compound having antibacterial activity. In particular embodiments, the vancomycin class compound is telavancin. In other embodiments the vancomycin class compound is a compound of formula (I).

The compounds of the invention are described in formulas in which hydrogen atoms are sometimes indicated with the letter H. These formulas include hydrogen isotopes in their naturally occurring abundances, as well as compounds in which one or more hydrogen atoms of the compound is isotopically enriched (e.g., 85%, 90%, 95%, or 98%) with deuterium. Such enrichments can be made, for example, using the semi-synthetic approaches described herein wherein the starting material is extracted from an organism grown in the presence of deuterated water, or feed with deuterated amino acids. Alternatively, isotopic enrichment can be achieved by employing a deuterated substituent in one or more reactions of any precursor to the compound of the invention.

By “acyl carnitine” is meant a chemical moiety with the formula:

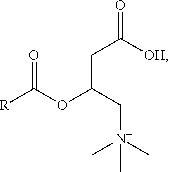

and salts thereof, wherein R is a partially-saturated straight chain or branched hydrocarbon group having between 8 and 26 carbon atoms. Acyl carnitines are derived carnitine (D or L form, or a mixture thereof) and a fatty acid. The acyl carnitine can be an ester of a fatty acid having 16 carbon atoms and 0, 1 or 2 double bonds (C16:0; C16:1 and C16:2), those with 18 carbon atoms and 1, 2 or 3 double bonds (C18:1; C18:2; and C18:3), those with 20 carbon atoms and 1, 2 or 4 double bonds (C20:1; C20:2; and C20:4), or those with 22 carbon atoms and 4, 5 or 6 double bonds (C22:4; C22:5 and C22:6). Acyl carnitines include, without limitation, 4, 7, 10, 13, 16, 19 docosahexanoyl carnitine, oleoyl carnitine, palmitoyl carnitine, decanoyl carnitine, dodecanoyl carnitine, myristoyl carnitine, and stearoyl carnitine.

By “additive” is meant those components of a pharmaceutical composition containing a vancomycin class compound in oral dosage form which increase the oral bioavailability of the drug when orally administered simultaneously with the drug. Additives of the invention include sugar esters, alkyl saccharides, acyl carnitines, glycerides, chitosan and derivatives thereof, amido fatty acids, fatty acids and salts or esters thereof, polyethylene glycol alkyl ethers, poly-D-lysine, N-acetyl-L-cystine, and combinations thereof.

As used herein, the term “vancomycin class compound” refers to an antibiotic glycopeptide including a backbone formed from a heptapeptide in which the amino acid residues at positions 2, 4, and 6 are cross-linked via two biaryl ether linkages to form two 16-membered macrocycles and the amino acid residues at positions 5 and 7 are cross-linked via a biphenyl ring to form a 12-membered macrocycle. The backbone for this class of compound is shown below. Vancomycin class compounds

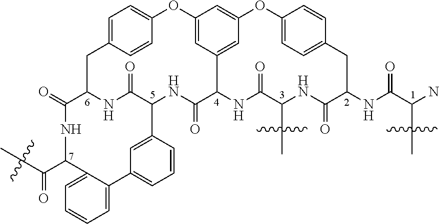 [SMILES: *C(=O)C1NC(=O)C2Cc3ccc(cc3)Oc3cc4cc(c3)C(NC(=O)C(*)NC(=O)C(NC(=O)C(*)N)Cc3ccc(cc3)O4)C(=O)NC(C(=O)N2)c2cccc(c2)-c2ccccc21]

backbone for a vancomycin class compounds
include, without limitation, vancomycin, oritavancin, teicoplanin, dalbavancin, telavancin, eremomycin, and chloroeremomycin.

As used herein, by “increase the oral bioavailability” is meant at least 25%, 50%, 75%, 100%, or 300% greater bioavailability of an orally administered vancomycin class compound, as a measured average of AUC in canine subjects for an oral dosage form of the invention including a vancomycin class compound formulated with one or more additives in comparison to the same vancomycin class compound formulated without any additives. For these studies the subjects have gastrointestinal tracts that have not been surgically manipulated in a manner that would alter the oral bioavailability of a vancomycin class compound.

In the generic descriptions of compounds of this invention, the number of atoms of a particular type in a substituent group is generally given as a range. For example, an alkyl group containing from 1 to 10 carbon atoms. Reference to such a range is intended to include specific references to groups having each of the integer number of atoms within the specified range. For example, an alkyl group from 1 to 10 carbon atoms includes each of C₁, C₂, C₃, C₄, C₅, C₆, C₇, C₈, C₉, and C₁₀. Other numbers of atoms and other types of atoms are indicated in a similar manner.

By “C₁₋₁₀ alkyl” is meant a branched or unbranched hydrocarbon group having from 1 to 10 carbon atoms. A C₁₋₁₀ alkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl. C₁₋₁₀ alkyls include, without limitation, adamantyl, methyl, ethyl, n-propyl, isopropyl, cyclopropyl, cyclopropylmethyl, n-butyl, iso-butyl, sec-butyl, tert-butyl, cyclobutyl, n-pentyl, cyclopentyl, n-hexyl, cyclohexyl, heptyl, and octyl, among others. Alkyl groups of other lengths are similarly branched or unbranched and substituted or unsubstituted.

By “C₂₋₁₀ alkenyl” is meant a branched or unbranched hydrocarbon group containing one or more double bonds and having from 2 to 10 carbon atoms. A C₂₋₁₀ alkenyl may optionally include monocyclic or polycyclic rings, in which each ring desirably has from three to six members. The C₂₋₁₀ alkenyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl. C₂₋₁₀ alkenyls include, without limitation, vinyl, allyl, 2-cyclopropyl-1-ethenyl, 1-propenyl, 1-butenyl, 2-butenyl, 3-butenyl, 2-methyl-1-propenyl, and 2-methyl-2-propenyl. Alkenyl groups of other lengths are similarly branched or unbranched and substituted or unsubstituted.

By “C₂₋₁₀ alkynyl” is meant a branched or unbranched hydrocarbon group containing one or more triple bonds and having from 2 to 10 carbon atoms. A C₂₋₁₀ alkynyl may optionally include monocyclic, bicyclic, or tricyclic rings, in which each ring desirably has seven or eight members. The C₂₋₁₀ alkynyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl. C₂₋₁₀ alkynyls include, without limitation, ethynyl, 1-propynyl, 2-propynyl, 1-butynyl, 2-butynyl, and 3-butynyl. Alkynyl groups of other lengths are similarly branched or unbranched and substituted or unsubstituted.

By “C₂₋₁₀ heterocyclyl” is meant a stable 5- to 7-membered monocyclic or 7- to 14-membered bicyclic heterocyclic ring which is saturated partially unsaturated or unsaturated (aromatic), and which consists of 2 to 6 carbon atoms and 1, 2, 3 or 4 heteroatoms independently selected from N, O, and S and including any bicyclic group in which any of the above-defined heterocyclic rings is fused to a benzene ring. The heterocyclyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl. The nitrogen and sulfur heteroatoms may optionally be oxidized. The heterocyclic ring may be covalently attached via any heteroatom or carbon atom which results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. Preferred 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, and tetrazolyl.

By “C₆₋₁₂ aryl” is meant an aromatic group having a ring system comprised of carbon atoms with conjugated π electrons (e.g., phenyl, biphenyl, napthyl, etc.). The aryl group has from 6 to 12 carbon atoms. Aryl groups may optionally include monocyclic, bicyclic, or tricyclic rings, in which each ring desirably has five or six members. The aryl group may be substituted or unsubstituted. Exemplary substituents include alkyl, hydroxy, alkoxy, aryloxy, sulfhydryl, alkylthio, arylthio, halide, fluoroalkyl, carboxyl, hydroxyalkyl, carboxyalkyl, amino, aminoalkyl, monosubstituted amino, disubstituted amino, and quaternary amino. Aryl groups of other sizes are similarly substituted or unsubstituted.

By “C₇₋₁₆ alkaryl” is meant a C₁₋₄ alkyl substituted by a C₆₋₁₂ aryl group (e.g., benzyl, phenethyl, or 3,4-dichlorophenethyl) having from 7 to 16 carbon atoms. Alkaryl groups of other lengths are similarly branched or unbranched and substituted or unsubstituted.

By “C₃₋₁₀ alkheterocyclyl” is meant an alkyl substituted heterocyclic group having from 3 to 10 carbon atoms in addition to one or more heteroatoms (e.g., 3-furanylmethyl, 2-furanylmethyl, 3-tetrahydrofuranylmethyl, or 2-tetrahydrofuranylmethyl).

By “C₁₋₁₀ heteroalkyl” is meant a branched or unbranched alkyl, alkenyl, or alkynyl group having from 1 to 10 carbon atoms in addition to 1, 2, 3 or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, hydroxyalkyl, carboxyalkyl, and carboxyl. Examples of C₁₋₁₀ heteroalkyls include, without limitation, polyamines, methoxymethyl, and ethoxyethyl. Heteroalkyl groups of other lengths are similarly branched or unbranched and substituted or unsubstituted.

By “halide” is meant bromide, chloride, iodide, or fluoride. By “fluoroalkyl” is meant an alkyl group that is substituted with a fluorine atom.

By “perfluoroalkyl” is meant an alkyl group consisting of only carbon and fluorine atoms.

By “carboxyalkyl” is meant a chemical moiety with the formula —(R)—COOH, wherein R is selected from C₁₋₁₀ alkyl, C₂₋₁₀ alkenyl, C₂₋₁₀ alkynyl, C₂₋₁₀ heterocyclyl, C₆₋₁₂ aryl, C₇₋₁₆ alkaryl, C₃₋₁₀ alkheterocyclyl, or C₁₋₁₀ heteroalkyl.

By “hydroxyalkyl” is meant a chemical moiety with the formula —(R)—OH, wherein R is selected from C₁₋₁₀ alkyl, C₂₋₁₀ alkenyl, C₂₋₁₀ alkynyl, C₂₋₁₀ heterocyclyl, C₆₋₁₂ aryl, C₇₋₁₆ alkaryl, C₃₋₁₀ alkheterocyclyl, or C₁₋₁₀ heteroalkyl.

By “alkoxy” is meant a chemical substituent of the formula —OR, wherein R is selected from C₁₋₁₀ alkyl, C₂₋₁₀ alkenyl, C₂₋₁₀ alkynyl, C₂₋₁₀ heterocyclyl, C₆₋₁₂ aryl, C₇₋₁₆ alkaryl, C₃₋₁₀ alkheterocyclyl, or C₁₋₁₀ heteroalkyl.

By “aryloxy” is meant a chemical substituent of the formula —OR, wherein R is a C₆₋₁₂ aryl group.

By “alkylthio” is meant a chemical substituent of the formula —SR, wherein R is selected from C₁₋₁₀ to alkyl, C₂₋₁₀ alkenyl, C₂₋₁₀ alkynyl, C₂₋₁₀ heterocyclyl, C₆₋₁₂ aryl, C₇₋₁₆ alkaryl, C₃₋₁₀ alkheterocyclyl, and C₁₋₁₀ heteroalkyl.

By “arylthio” is meant a chemical substituent of the formula —SR, wherein R is a C₆₋₁₂ aryl group.

By “quaternary amino” is meant a chemical substituent of the formula —(R)—N(R′)(R″)(R′″), wherein R, R′, R″, and R′″ are each independently an alkyl, alkenyl, alkynyl, or aryl group. R may be an alkyl group linking the quaternary amino nitrogen atom, as a substituent, to another moiety. The nitrogen atom, N, is covalently attached to four carbon atoms of alkyl and/or aryl groups, resulting in a positive charge at the nitrogen atom.

By “increased oral bioavailability” is meant the fraction of drug absorbed following oral administration to a subject is increased for the compound of the invention in comparison to vancomycin orally administered under the same conditions (e.g., fasted or fed). The compounds of the invention can exhibit at least 25%, 50%, 100%, 200%, or 300% greater oral bioavailability than vancomycin.

As used herein, the term “treating” refers to administering a pharmaceutical composition for prophylactic and/or therapeutic purposes. To “prevent disease” refers to prophylactic treatment of a subject who is not yet ill, but who is susceptible to, or otherwise at risk of, a particular disease. To “treat disease” or use for “therapeutic treatment” refers to administering treatment to a subject already suffering from a disease to improve or stabilize the subject's condition. Thus, in the claims and embodiments, treating is the administration to a subject either for therapeutic or prophylactic purposes.

As used herein, the terms “an amount sufficient” and “sufficient amount” refer to the amount of a vancomycin class compound required to treat or prevent an infection. The sufficient amount used to practice the invention for therapeutic or prophylactic treatment of conditions caused by or contributed to by an infection varies depending upon the manner of administration, the type of infection, the age, body weight, and general health of the subject. Ultimately, the attending physician or veterinarian will decide the appropriate amount and dosage regimen. Such amount is referred to as a “sufficient” amount.

The term “unit dosage form” refers to physically discrete units suitable as unitary dosages, such as a pill, tablet, caplet, hard capsule, soft capsule, a premeasured reconstitutable powder or liquid, or sachet, each unit containing a predetermined quantity of a vancomycin class compound of the invention. By “hard capsule” is meant a capsule that includes a membrane that forms a two-part, capsule-shaped, container capable of carrying a solid or liquid payload of drug and excipients. By “soft capsule” is meant a capsule molded into a single container carrying a liquid or semisolid payload of drug and excipients.

By “bacterial infection” is meant the invasion of a host by pathogenic bacteria. For example, the infection may include the excessive growth of bacteria that are normally present in or on the body of a subject or growth of bacteria that are not normally present in or on a subject. More generally, a bacterial infection can be any situation in which the presence of a bacterial population(s) is damaging to a host body. Thus, a subject is “suffering” from a bacterial infection when an excessive amount of a bacterial population is present in or on the subject's body, or when the presence of a bacterial population(s) is damaging the cells or other tissue of the subject.

As used herein, the term “prodrug” refers to prodrugs of compounds of the invention that include one or more labile groups which are removed following administration to a subject, resulting in a compound of formula (I). Prodrugs include hydrolysable groups, such as esters and carbonates, among other hydrolyzable bonds.

Other features and advantages of the invention will be apparent from the following detailed description, the drawings, and the claims.

### BRIEF DESCRIPTION OF THE DRAWINGS

FIG. 1 is a table depicting the MIC₅₀ values for test compounds when tested against a well-characterized collection of Gram-positive organisms.

FIG. 2 is a scheme depicting how the compounds of the invention can be synthesized.

FIG. 3 is a scheme depicting how the compounds of the invention can be synthesized.

### DETAILED DESCRIPTION

The invention features compounds which have been modified to be suitable for oral administration and/or modified to increase their antimicrobial potency.

### Compounds

Compounds of the invention include compounds of formula (I), formula (II), formulas (IIIa)-(IIIf) (shown below), formulas (IVa)-(IVf) (shown below), formulas (Va)-(Vf) (shown below), compounds of formula (VI), compounds of formula (VII), and compounds of formula (VIII). These compounds can be synthesized, for example, as described in the examples by coupling functionalized or unfunctionalized glycopeptides with the appropriate acyl, alkyl and/or amino groups under standard reaction conditions.

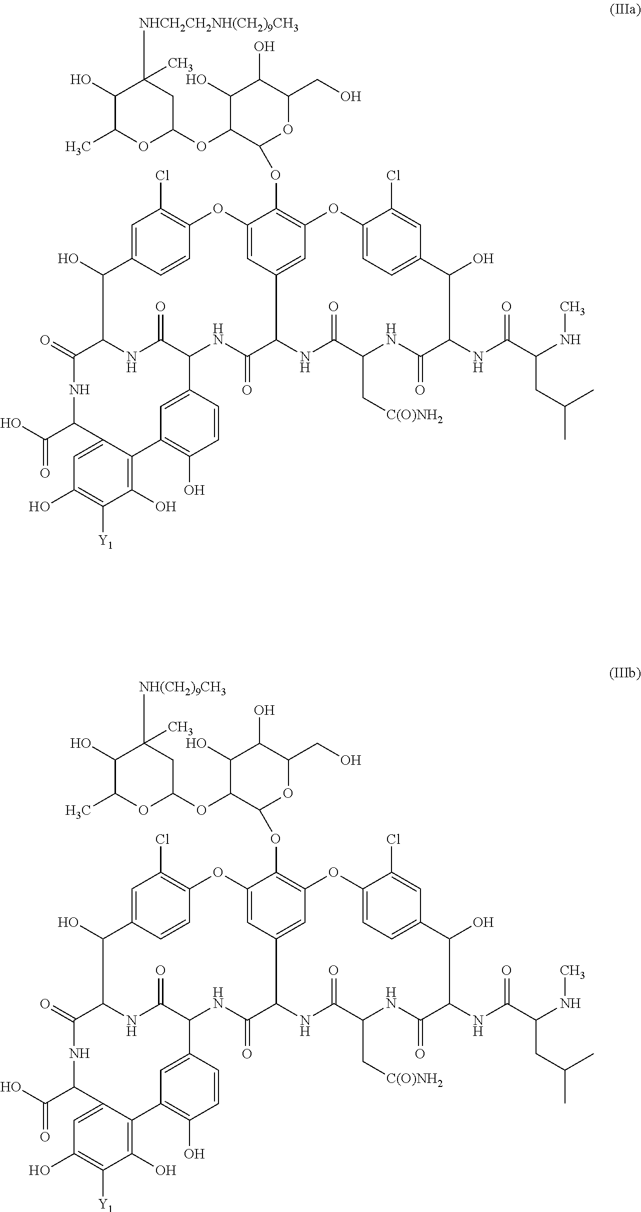

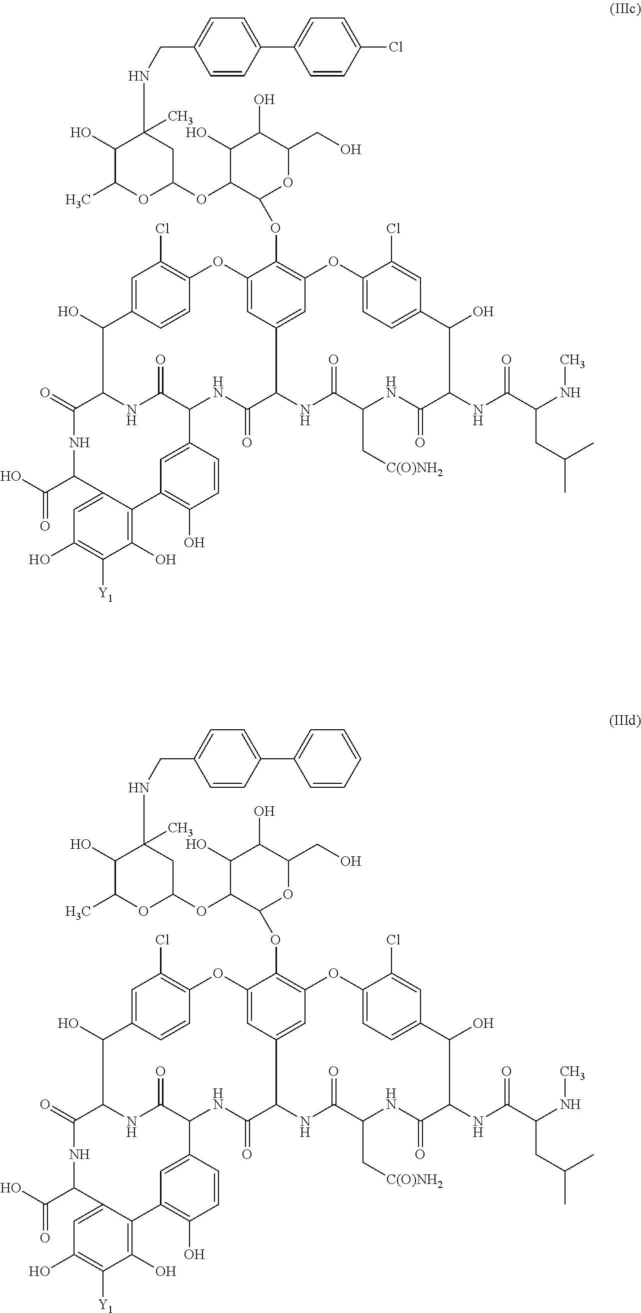

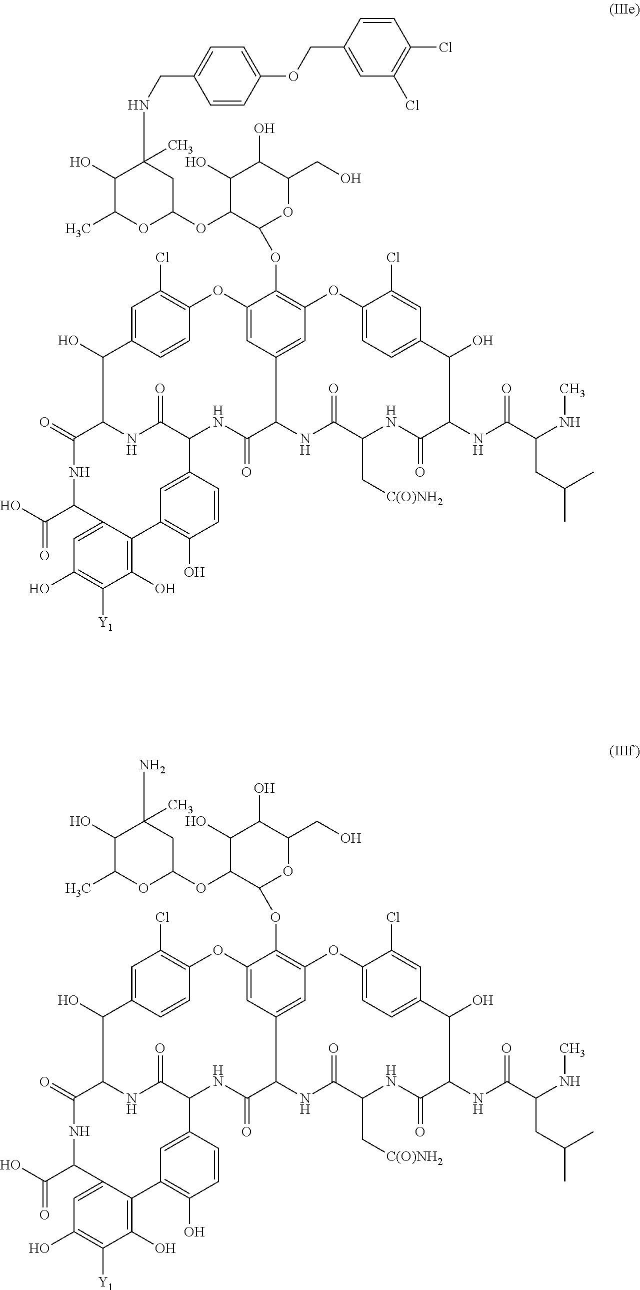

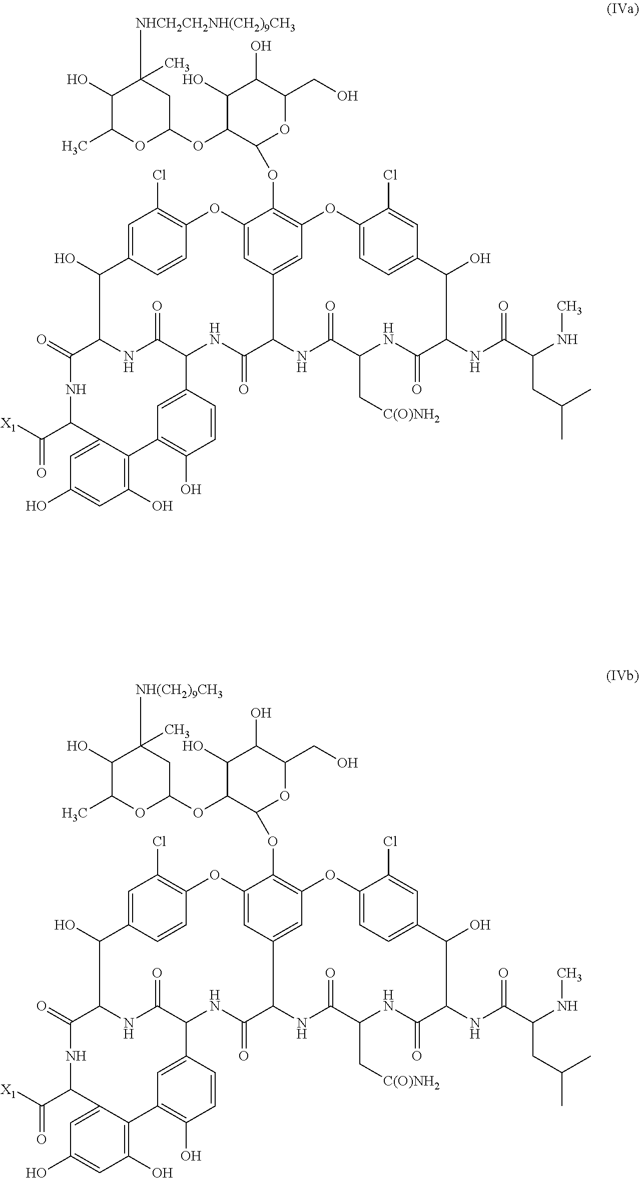

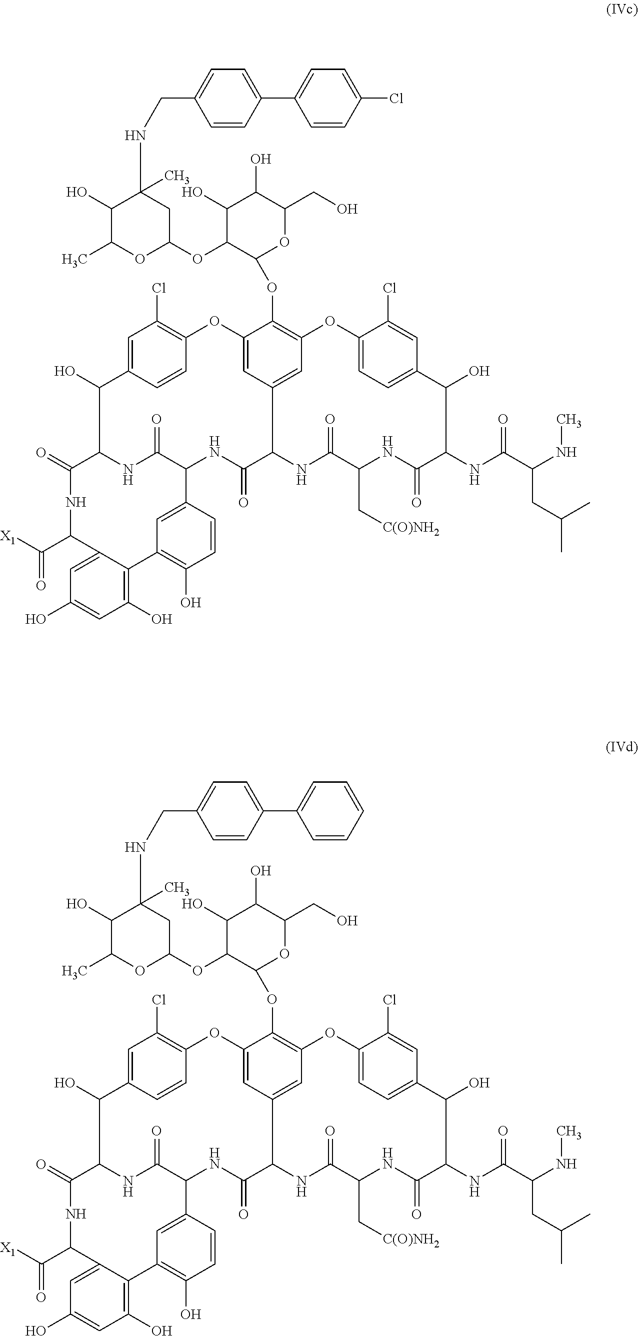

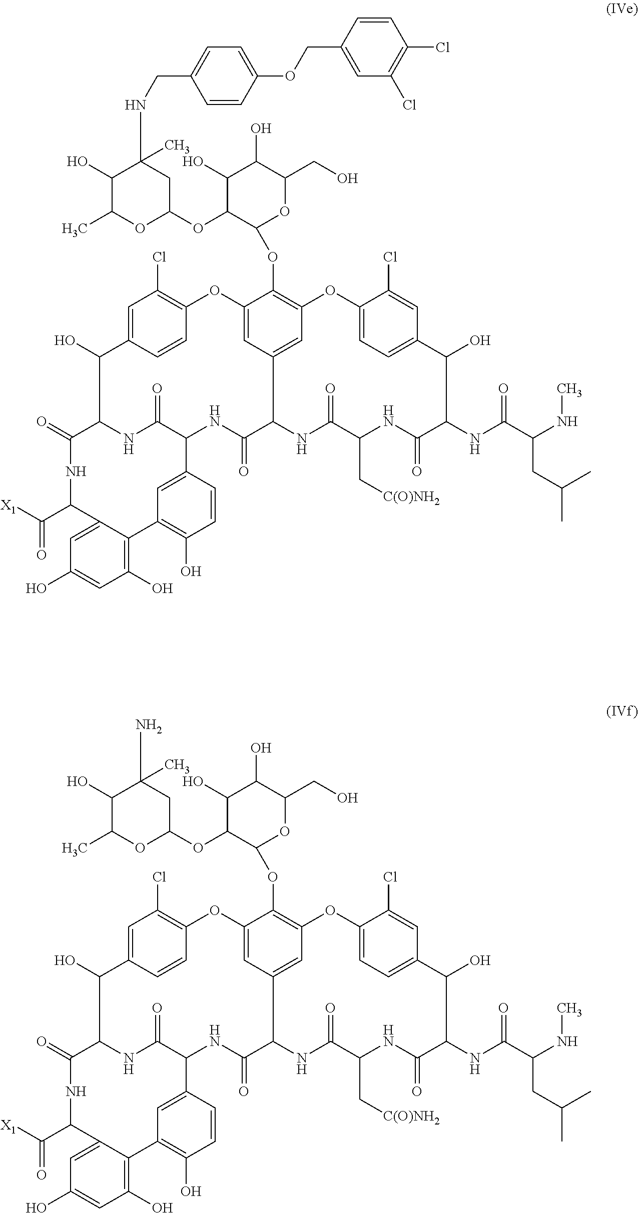

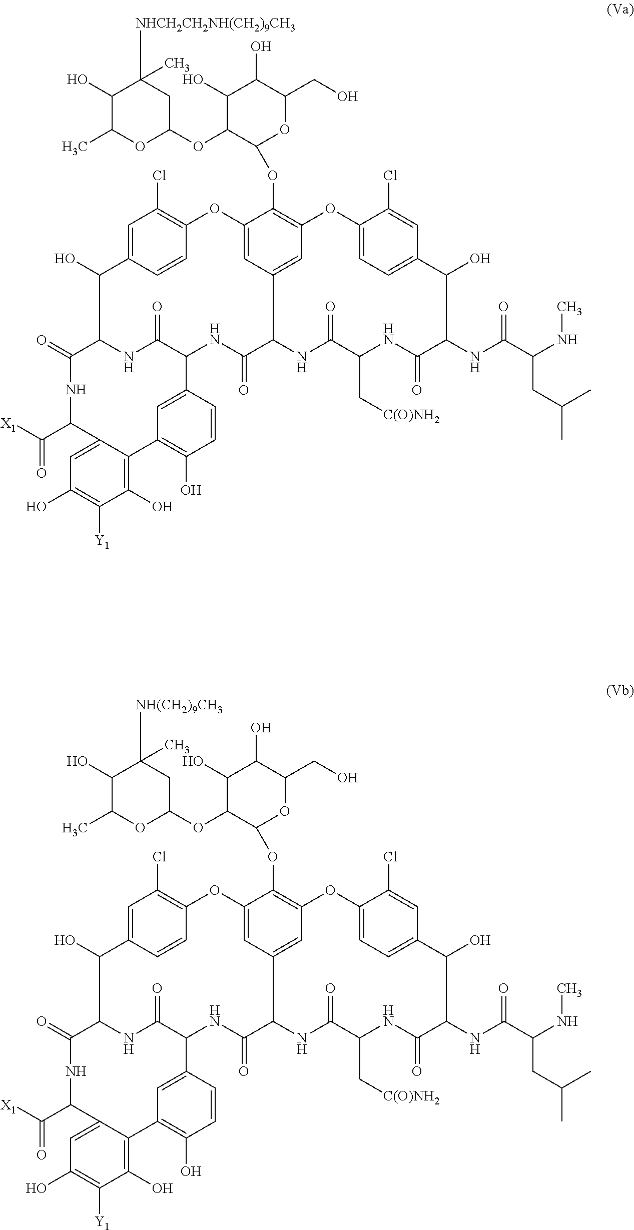

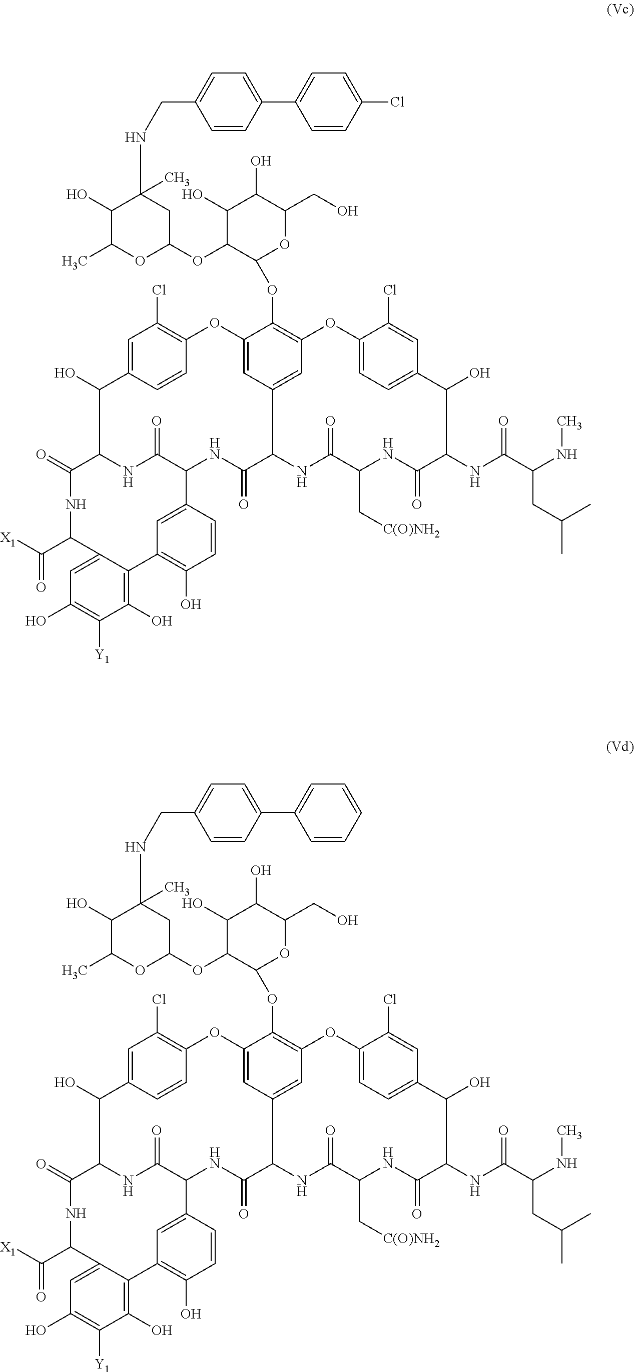

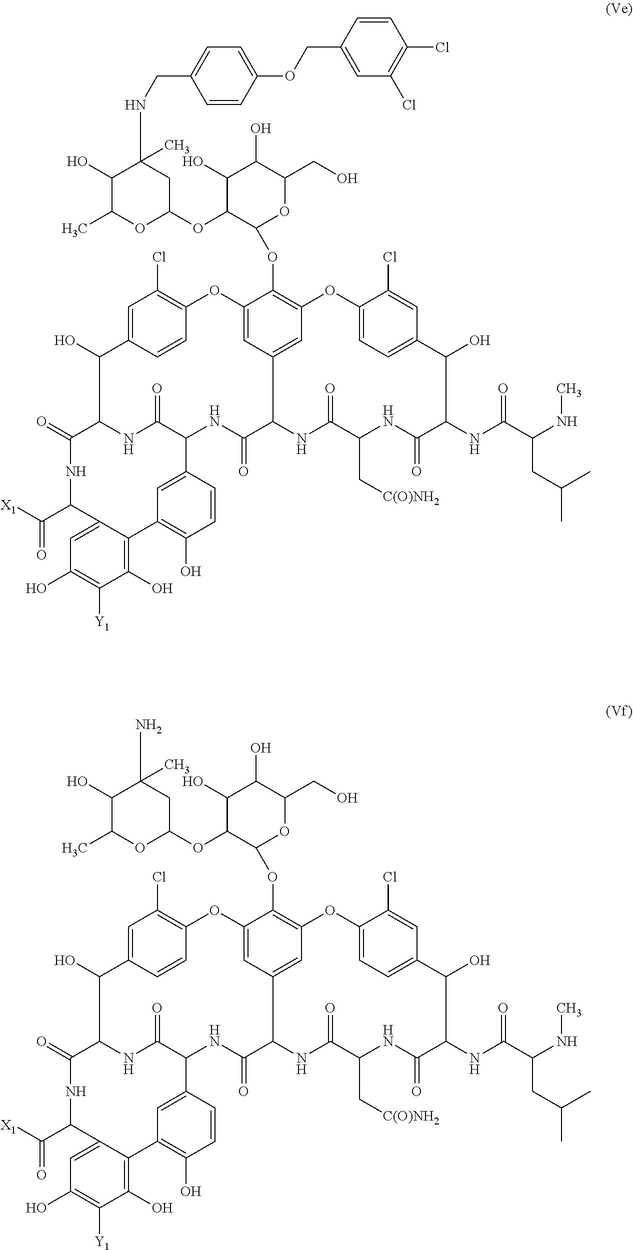

In formulas (IIIa)-(IIIf), (IVa)-(IVf), and (Va)-(Vf), X₁ and Y₁ are as defined in formula (I). Typically, the semi-synthetic vancomycin class compounds of the invention are made by modifying the naturally occurring vancomycin scaffold. For example, starting from vancomycin, the amine bearing saccharide group, vancosamine, can be alkylated via a reductive amination of a substituent (e.g., an alkyl, heteroalkyl, or aryl group). Alternatively, the C-terminal carboxylate (i.e., position X₁) can be amidated using standard amide coupling synthetic methods. Substitutions can also be made at the resorcinol ring (i.e., position Y₁) using Mannich chemistry to incorporate an aminomethyl substituent which may then be further modified.

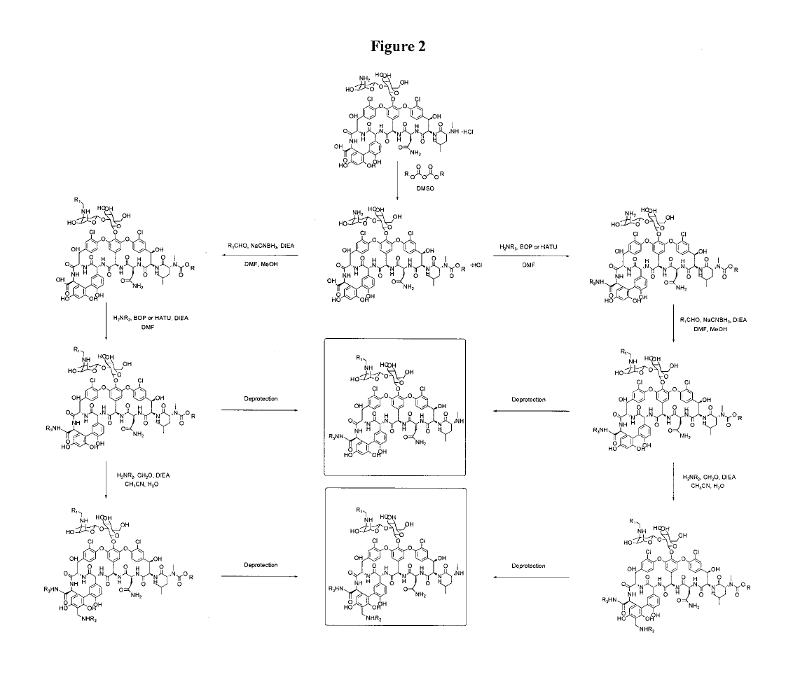

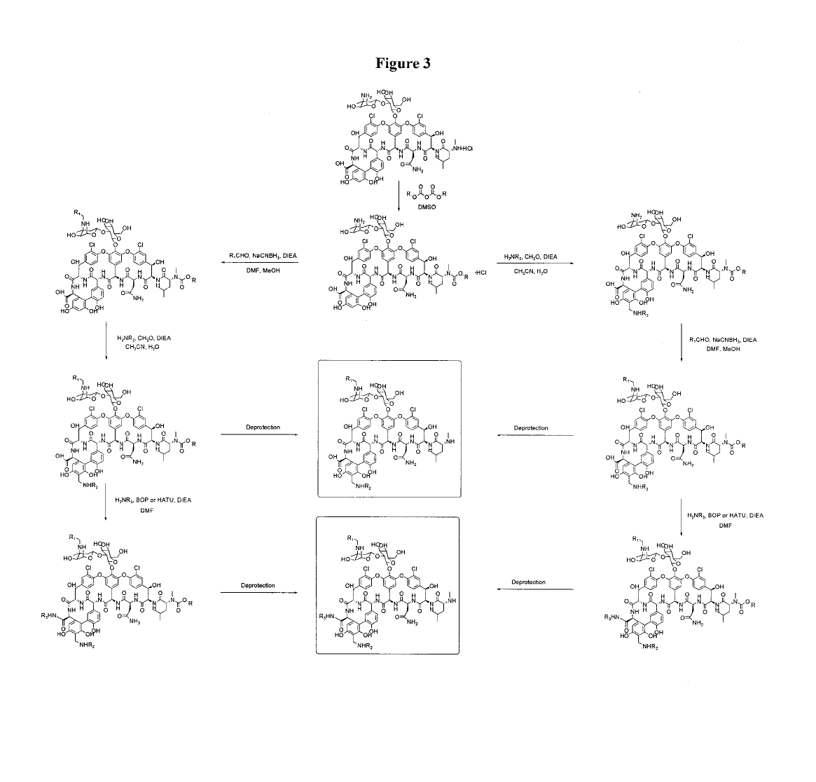

The compounds of the invention can be made using the general synthetic schemes depicted in FIGS. 2 and 3, and using methods analogous to those described for compound 1.

For semi-synthetic approaches to vancomycin class compounds of the invention, the stereochemistry of the glycopeptide will be dictated by the starting material. Thus, the stereochemistry of vancomycin derivatives will typically have the same stereochemistry as the naturally occurring vancomycin scaffold. Accordingly, the vancomycin class compounds can be prepared from naturally occurring starting materials or their derivatives (e.g., vancomycin, oritavancin, eremomycin, telavancin, and chloroeremomycin) and share the same stereochemical configuration at each of the saccharide groups and amino acid residues found in the naturally occurring glycopeptides from which the compounds of the invention are synthesized.

### Therapy and Formulation

The invention features pharmaceutical formulations for oral administration of a vancomycin class compound. The formulations can include an additive selected from sugar esters, alkyl saccharides, acyl carnitines, glycerides, polyethylene glycol alkyl ethers, chitosan and derivatives thereof, amido fatty acids, fatty acids and salts or esters thereof, poly-D-lysine, N-acetyl-L-cystine, and combinations thereof. These additives can increase the oral bioavailability of vancomycin class compounds. Further details are provided below. In some instances, the commercial product and supplier for a particular additive is provided in parentheses following the identification of the additive. Typically the additive, or combination of additives, is from 10 to 90% (w/w) of the oral dosage form.

Sugar Esters

Sugar Esters that can be used in the oral dosage forms of the invention include, without limitation, sucrose distearate (Crodesta F-10/Croda); sucrose distearate/monostearate (Crodesta F-110/Croda); sucrose dipalmitate; sucrose monostearate (Crodesta F-160/Croda); sucrose monopalmitate (SUCRO ESTER 15/Gattefosse); sucrose monocaprate, and sucrose monolaurate (saccharose monolaurate 1695/Mitsubisbi-Kasei). In particular embodiments, the vancomycin class compound is formulated with a C₈₋₁₂ fatty acid ester of a sugar, such as n-decanoylsucrose (EMD).

Alkyl Saccharides

Alkyl saccharides can be used in the oral dosage forms of the invention. Alkyl saccharides are sugar ethers of a hydrophobic alkyl group (e.g., typically from 9 to 24 carbon atoms in length). Alkyl saccharides include alkyl glycosides and alkyl glucosides. In particular embodiments, the vancomycin class compound is formulated with a C₈₋₁₄ alkyl ether of a sugar. Alkyl glycosides that can be used in the oral dosage forms of the invention include, without limitation, C₈₋₁₄ alkyl (e.g., octyl-, nonyl-, decyl-, undecyl-, dodecyl-, tridecyl-, or tetradecyl-) ethers of α or β-D-maltoside, -glucoside or -sucroside, alkyl thiomaltosides, such as heptyl, octyl, dodecyl-, tridecyl-, and tetradecyl-β-D-thiomaltoside; alkyl thioglucosides, such as heptyl- or octyl 1-thio α- or β-D-glucopyranoside; alkyl thiosucroses; and alkyl maltotriosides. For example, the vancomycin class compound can be formulated with octyl maltoside, dodecyl maltoside, tridecyl maltoside, tetradecyl maltoside, sucrose mono-dodecanoate, sucrose mono-tridecanoate, or sucrose mono-tetradecanoate. Alkyl glucosides that can be used in the oral dosage forms of the invention include, without limitation, C₈₋₁₄ alkyl (e.g., octyl-, nonyl-, decyl-, undecyl-, dodecyl-, tridecyl-, or tetradecyl-) ethers of glucoside, such as dodecyl glucoside or decyl glucoside.

Acyl Carnitines

Acyl carnitines can be used in the oral dosage forms of the invention, in either their zwitter ion form or salt form. Acyl carnitines can be derived carnitine (D or L form, or a mixture thereof) and a fatty acid including, without limitation, fatty acids having 16 carbon atoms and 0, 1 or 2 double bonds (C16:0; C16:1 and C16:2), those with 18 carbon atoms and 1, 2 or 3 double bonds (C18:1; C18:2; and C18:3), those with 20 carbon atoms and 1, 2 or 4 double bonds (C20:1; C20:2; and C20:4) and those with 22 carbon atoms and 4, 5 or 6 double bonds (C22:4; C22:5 and C22:6). Exemplary acyl carnitines which are useful additives in the formulations of the invention include octyl carnitine, oleoyl carnitine, palmitoyl carnitine, decanoyl carnitine, dodecanoyl carnitine, myristoyl carnitine, and stearoyl carnitine.

Glycerides

Glycerides can be used in the oral dosage forms of the invention. Glycerides are fatty acid mono-, di-, and tri-esters of glycerol. A variety of glycerides can be used in the oral dosage forms of the invention. Glycerides include saturated and unsaturated monoglycerides, diglyceridies (1,2- and 1,3-diglycerides), and triglycerides, with mixed and unmixed fatty acid composition. Each glyceride is herein designated as (Cn:m), where n is the length of the fatty acid side chain and m is the number of double bonds (cis- or trans-) in the fatty acid side chain. Examples of commercially available monoglycerides include: monocaprylin (C8; i.e., glyceryl monocaprylate) (Larodan), monocaprin (C10; i.e., glyceryl monocaprate) (Larodan), monolaurin (C12; i.e., glyceryl monolaurate) (Larodan), monopalmitolein (C16:1) (Larodan), glyceryl monomyristate (C14) (Nikkol MGM, Nikko), glyceryl monooleate (C18:1) (PECEOL, Gattefosse), glyceryl monooleate (Myverol, Eastman), glycerol monooleate/linoleate (OLICINE, Gattefosse), glycerol monolinoleate (Maisine, Gattefosse), and monoelaidin (C18:1) (Larodan). Examples commercially available diglycerides include: glyceryl laurate (Imwitor® 312, Huls), glyceryl caprylate/caprate (Capmul® MCM, ABITEC), caprylic acid diglycerides (Imwitor® 988, Huls), caprylic/capric glycerides (Imwitor® 742, Huls), dicaprylin (C8) (Larodan), dicaprin (C10) (Larodan), dilaurin (C12) (Larodan), glyceryl dilaurate (C12) (Capmul® GDL, ABITEC). Examples commercially available triglycerides include: tricaprylin (C8; i.e., glyceryl tricaprylate) (Larodan), tricaprin (C10; i.e., glyceryl tricaprate) (Larodan), trilaurin (C12; i.e., glyceryl trilaurate) (Larodan), dimyristin (C14) (Larodan), dipalmitin (C16) (Larodan), distearin (Larodan), glyceryl dilaurate (C12) (Capmul® GDL, ABITEC), glyceryl dioleate (Capmul® GDO, ABITEC), glycerol esters of fatty acids (GELUCIRE 39/01, Gattefosse), dipalmitolein (C16:1) (Larodan), 1,2 and 1,3-diolein (C18:1) (Larodan), dielaidin (C18:1) (Larodan), and dilinolein (C18:2) (Larodan). Glycerides which can be used in the oral dosage forms of the invention include, for example, Capmul MCM C10 (Mono/Di C10 glycerides) and Captex 1000 (C10 tri glycerides 95%), branched fatty acid glycerides, and cyclic glycerides.

Polyethylene Glycol Alkyl Ethers

Ethers of polyethylene glycol and alkyl alcohols can be used in the oral dosage forms of the invention. Preferred polyethylene glycol alkyl ethers include Laureth 9, Laureth 12 and Laureth 20. Other polyethylene glycol alkyl ethers include, without limitation, PEG-2 oleyl ether, oleth-2 (Brij 92/93, Atlas/ICI); PEG-3 oleyl ether, oleth-3 (Volpo 3, Croda); PEG-5 oleyl ether, oleth-5 (Volpo 5, Croda); PEG-10 oleyl ether, oleth-10 (Volpo 10, Croda, Brij 96/97 12, Atlas/ICI); PEG-20 oleyl ether, oleth-20 (Volpo 20, Croda, Brij 98/99 15, Atlas/ICI); PEG-4 lauryl ether, laureth-4 (Brij 30, Atlas/ICI); PEG-9 lauryl ether; PEG-23 lauryl ether, laureth-23 (Brij 35, Atlas/ICI); PEG-2 cetyl ether (Brij 52, ICI); PEG-10 cetyl ether (Brij 56, ICI); PEG-20 cetyl ether (Brij 58, ICI); PEG-2 stearyl ether (Brij 72, ICI); PEG-10 stearyl ether (Brij 76, ICI); PEG-20 stearyl ether (Brij 78, ICI); and PEG-100 stearyl ether (Brij 700, ICI).

Chitosan and Derivatives Thereof

Chitosan and derivatives thereof can be used in the oral dosage forms of the invention. Chitosan is prepared by the deacetylation of chitin. For use in the formulations of the invention, the degree of deacetylation, which represents the proportion of N-acetyl groups which have been removed through deacetylation, should be in the range of from about 40 to about 100%, (e.g., 60 to about 96% or 70 to 95%). Desirably, the chitosan, or chitosan derivative, should have a molecular weight of from about 5,000 to about 1,000,000 Da (e.g., from about 10,000 to about 800,000 Da, from about 15,000 to about 600,000 Da, or from 30,000 or 50,000 to about 600,000 Da). Chitosan derivatives include pharmaceutically acceptable organic and inorganic salts (e.g., nitrate, phosphate, acetate, hydrochloride, lactate, citrate and glutamate salts, among others). Chitosan derivatives can be prepared by bonding moieties to the hydroxyl or amino groups of chitosan and may confer the polymer with changes in properties such as solubility characteristics and charge density. Examples include O-alkyl ethers of chitosan and O-acyl esters of chitosan. Other examples of chitosan derivatives include carboxymethyl chitosan (see Thanou et al, J. Pharm. Sci., 90:38 (2001)) and N-carboxymethyl chitosan derivatives, trimethylchitosan (see Thanou et al, Pharm. Res., 17:27 (2000)), thiolated chitosans (see Bernkop-Schnurch et al, Int. J. Pharm., 260:229 (2003)), piperazine derivatives (see PCT Publication No. WO2007/034032 and Holappa et al, Macromol. Biosci., 6:139 (2006)), PEG-conjugated chitosan (see PCT Publication No. WO 99/01498), and those derivatives disclosed in Roberts, Chitin Chemistry, MacMillan Press Ltd., London (1992). Exemplary chitosan and chitosan derivatives which are useful additives in the formulations of the invention include chitosan, trimethylchitosan, and chitosan-4-thio-butylamidine (see Sreenivas et al., International Journal of PharmTech Research 1:670 (2009)).

Amido Fatty Acids

Amido fatty acids can be used in the oral dosage forms of the invention. Amido fatty acids are long chain amino acid amides of formula (XX), and salts thereof:

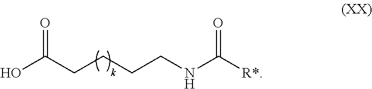

In formula (XX), k is an integer from 4 to 10 and R* is C₅₋₈ alkyl, C₆₋₁₂ aryl, C₇₋₁₆ alkaryl, C₃₋₁₀ alkheterocyclyl, and C₂₋₁₀ heterocyclyl. Amido fatty acids include those described in U.S. Pat. No. 5,650,386, incorporated herein by reference. Exemplary amido fatty acids which are useful additives in the formulations of the invention include sodium N-[8-(2-hydroxybenzoyl)amino]caprylate.

The invention features compositions and methods for treating or preventing a disease or condition associated with a bacterial infection by administering a compound of the invention. Compounds of the present invention may be administered by any appropriate route for treatment or prevention of a disease or condition associated with a bacterial infection. These may be administered to humans, domestic pets, livestock, or other animals with a pharmaceutically acceptable diluent, carrier, or excipient. When administered orally, these may be in unit dosage form. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, sublingual, buccal, aerosol, by suppositories, or oral administration.

Fatty Acids

Fatty acids which can be used in the oral dosage forms of the invention, in either their acid form, salt form, monoester form, or glyceride form, include caprylic acid (octanoic acid), pelargonic acid (nonanoic acid), capric acid (decanoic acid) and lauric acid (dodecanoic acid), and their primary hydroxyl forms 8-hydroxy octanoic acid, 9-hydroxy nonanoic acid, 10-hydroxy decanoic acid, and 12-hydroxy dodecanoic acid.

Fatty acids are commonly derived from natural fats, oils, and waxes by hydrolysis of esters and the removal of glycerol. Fatty acids can be titrated with sodium hydroxide solution using phenophthalein as an indicator to a pale-pink endpoint. This analysis is used to determine the free fatty acid content of fats; i.e., the proportion of the triglycerides that have been hydrolyzed.

Short-chain fatty acids such as acetic acid (pKa=4.76 in water) are miscible with water and dissociate to form acids. As its chain length increases, fatty acids do not substantially increase in pKa. However, as the chain length increases the solubility of fatty acids in water decreases very rapidly. However, most fatty acids that are insoluble in water will dissolve in warm ethanol.

Any alcohol can be used to produce a corresponding fatty acid ester. The alcohols may be polyalcohols such as ethylene glycol or glycerol. The alcohol may carry a permanent positive charge, which makes the ester mucoadhesive (that is, adhesive to musoca). Methods of esterification are well-known in the art (e.g., Fischer esterification in acid). Fatty acid esters include fatty acid ethyl esters and fatty acid methyl esters.

Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” (20th ed., ed. A. R. Gennaro, 2000, Lippincott Williams & Wilkins) Formulations for parenteral administration may, for example, contain excipients, sterile water, or saline, polyalkylene glycols such as polyethylene glycol, oils of vegetable origin, or hydrogenated napthalenes. Formulations for inhalation may contain excipients, for example, lactose, or may be aqueous solutions containing, for example, polyoxyethylene-9-lauryl ether, glycholate and deoxycholate, or may be oily solutions for administration in the form of nasal drops, or as a gel. The concentration of the compound in the formulation will vary depending upon a number of factors, including the dosage of the drug to be administered, and the route of administration.

The compound or combination may be optionally administered as a pharmaceutically acceptable salt, such as a non-toxic acid addition salts, alkali and alkaline earth salts (e.g., sodium, lithium, potassium, magnesium, or calcium salts), or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc). Formulations for oral use may also be provided in unit dosage form as chewable tablets, tablets, caplets, or capsules (i.e., as hard gelatin capsules wherein the active ingredient is mixed with an inert solid diluent, or as soft gelatin capsules wherein the active ingredient is mixed with water or an oil medium).

Formulations for oral use include liquid dosage forms, such as suspensions or sachets for reconstitution prior to oral administration.

The formulations can be administered to human subjects in therapeutically effective amounts. Typical dose ranges are from about 0.01 μg/kg to about 800 mg/kg of body weight per day. The preferred dosage of drug to be administered is likely to depend on such variables as the type and extent of the disorder, the overall health status of the particular subject, the specific compound being administered, the excipients used to formulate the compound, and its route of administration.

The compounds of the invention can be used to treat, for example, respiratory tract infections, acute bacterial otitis media, bacterial pneumonia, urinary tract infections, complicated infections, noncomplicated infections, pyelonephritis, intra-abdominal infections, deep-seated abcesses, bacterial sepsis, skin and skin structure infections, soft tissue infections, bone and joint infections, central nervous system infections, bacteremia, wound infections, peritonitis, meningitis, infections after burn, urogenital tract infections, gastro-intestinal tract infections, pelvic inflammatory disease, endocarditis, and other intravascular infections, complicated skin and skin structure infection, complicated intra-abdominal infection, hospital acquired pneumonia, ventilator associated pneumonia, pseudomembranous colitis, enterocolitis, infections associated with prosthetics or dialysis, preoperative antimicrobial prophylaxisand, and any other infection described herein.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

Analytical HPLC was performed using the following column(s) and conditions: Phenomenex Luna C18(2), 5 μm, 100 Å, 2.0×150 mm, 1-99% CH₃CN (0.1% TFA) in H₂O (0.1% TFA)/15 min. Preparative HPLC was performed using the following columns: Phenomenex Luna, 100 Å particle size, 10 micron pore size or Waters Nova-Pak HR C18, 6 μm, 60 Å, 19×300 mm. The following abbreviations are used in the examples below: min (minutes), hr (hours), mmol (millimole), μm (micron), Å (angstrom), THF (tetrahydrofuran), DMF (dimethylformamide), TLC (thin layer chromatography), HPLC (high performance liquid chromatography), LC/MS (liquid chromatography/mass spectrometry), TR (retention time on HPLC), ° C. (degrees celsius).

Compounds in the examples are identified by reference to the following structure, along with a description of groups R, X, and Y.

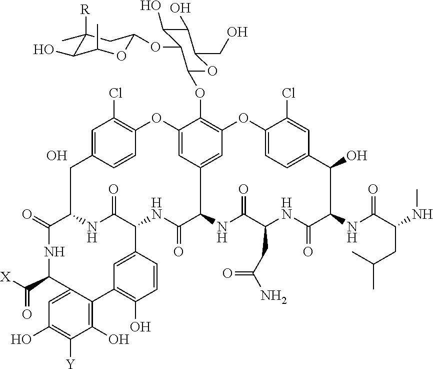

The compounds of the invention can be made using the general synthetic schemes depicted in FIGS. 2 and 3, and using methods analogous to those described for compound 1.

### Example 1

### Compounds of Formulas (IIIa) and (IVa)

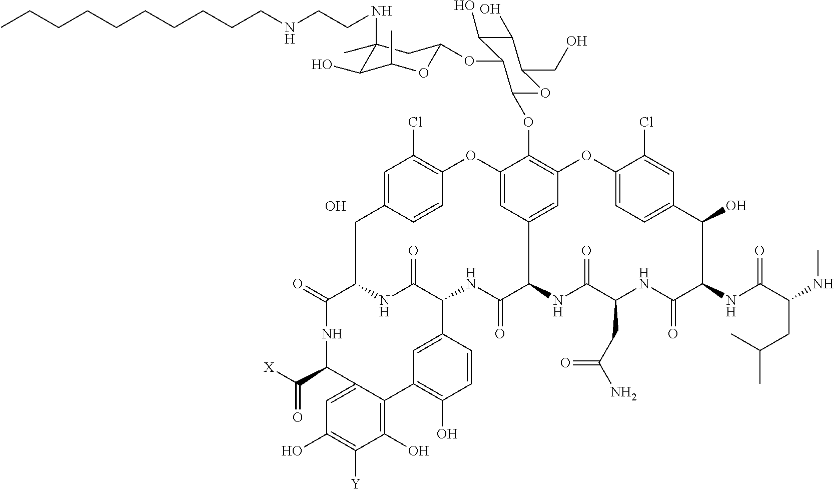

TABLE A

| Compound | X | Y |
|---|---|---|
| 1 | OH | CH₂NHCH₂CH₂(OCH₂CH₂)₂N(CH₃)₃ |
| 2 | NHCH₂CH₂(OCH₂CH₂)₂NH₂ | H |
| 3 | OH | CH₂NHCH₂CH₂(OCH₂CH₂)₂NH₂ |

### Example 2

### Synthesis of Compound 1

Compound 1 was synthesized as follows.

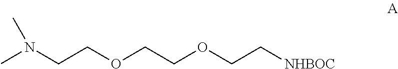

A solution of N-Boc-2,2′-(ethylenedioxy)diethylamine (19.06 g, 36.27 mmol) in dichloromethane (100 mL) was cooled under Argon with an ice/water bath to 0-5 C. Aqueous formaldehyde (37 wt %, 10.8 mL, 145.1 mmol) was added followed by sodium triacetoxyborohydride (30.7 g, 145 mmol) in portions and then allowed to stir for an additional 3 hrs. The reaction was diluted with water and quenched by dropwise addition of 10N NaOH to pH>12. The mixture was diluted with brine and transferred to a reparatory funnel. After separating the layers, the aqueous layer was back-extracted four times with dichloromethane. The combined organic layers were dried over anhydrous sodium sulfate, filtered, evaporated in vacuo and dried under high vacuum to provide a clear oil (21.90 g).

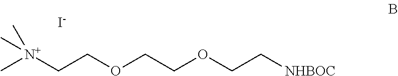

A solution of intermediate A (17.04 g, 61.6 mmol) in tetrahydrofuran (75 mL) was treated with methyl iodide (10 mL, 160 mmol) and heated at reflux under Argon for 16 hrs. The resulting slurry was cooled to ambient temperature, filtered, washed with cold THF and dried under high vacuum to provide a yellow solid (23.02 g).

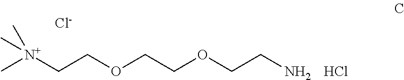

To a solution of hydrogen chloride (4.0 N in dioxane, 50 mL) under Argon was added Intermediate B (10.00 g, 31.4 mmol) using a water bath for cooling. The mixture was stirred for an additional 2 hrs., evaporated in vacuo and dried under high vacuum to provide a dark tacky semi-solid (8.49 g).

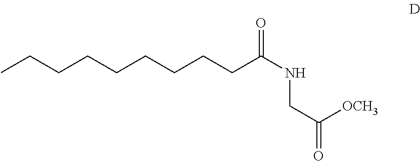 [SMILES: CCCCCCCCCC(=O)NCC(=O)OC]

A solution of decanoyl chloride (45 mL, 216.8 mmol) in DCM (340 mL) was cooled to 0-5° C. and treated with a solution of H-Gly-OMe-HCl (32.67 g, 260 mmol) and DIEA (83.1 mL, 2.2 eq) dissolved in DCM (340 mL). The reaction mixture was warmed to room temperature and stirred overnight. The reaction mixture was washed with 1M NaHSO₄ and NaHCO₃. The combined organic extracts were dried over MgSO₄, filtered, and concentrated to dryness under reduced pressure. The resultant white solid was slurried in hexanes and filtered. The filtrate was concentrated under reduced pressure and the resultant solid was slurried with hexanes and filtered with the rest of the solid. The bright white solid was dried under reduced pressure to provide of a bright white flaky solid (50.2 g).

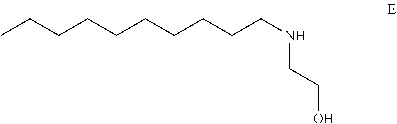

THF (250 mL) was slowly added to an argon purged flask containing LAH (16.8 g, 446 mmol). This suspension was brought to reflux and a solution of D (49.2 g, 202.5 mmol) in THF (200 mL) was added via addition funnel over one hour. After stiffing at reflux overnight, the reaction mixture was cooled with an ice bath. A solution of H₂O (˜17 mL) in THF (˜100 mL) was added dropwise while maintaining an internal temperature below 20° C. Additional THF (300 mL) was added in portions to maintain consistent stiffing. A 3 M solution of NaOH (˜17 mL) was added dropwise followed by the addition of water (˜52 mL). The reaction mixture was brought to reflux for about an hour at which point the solid in suspension turned completely white. The mixture was filtered through a Buchner funnel and the filtrate concentrated under reduced pressure to an oil. The residue is taken up in 300 mL EtOAc, dried over MgSO₄, and filtered. The solution was concentrated under reduced pressure to provide a clear oil (39 g) which turned to a white solid on standing.

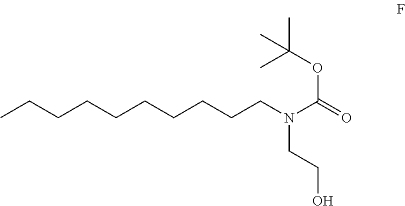 [SMILES: CCCCCCCCCCN(CCO)C(=O)OC(C)(C)C]

To a solution of the E (38 g, 188.7 mmol) in DCM (340 mL) at 0° C. was added DIEA (36.2 mL, 198 mmol). A solution of Boc₂O in DCM (100 mL) was added via addition funnel and stirred overnight. The reaction mixture was quenched by the addition of a 1M solution of NaHSO₄ (500 mL) and washed with NaHSO₄ (500 mL) and NaHCO₃. The combined organic extracts were dried over MgSO₄, filtered, and concentrated under reduced pressure to provide a clear liquid (58.3 g).

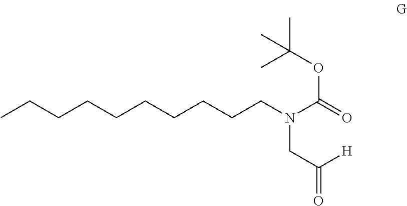 [SMILES: CCCCCCCCCCN(CC=O)C(=O)OC(C)(C)C]

To a stiffing solution of oxalyl chloride (48.6 mL, 566 mmol) in DCM (200 mL) at −50° C. was added a solution of DMSO (53.6 mL, 755 mmol) in DCM (55 mL) via addition funnel. After stiffing 15 minutes, a solution of F (56.9 g) in DCM (200 mL) was added to the reaction mixture over 30 minutes. The reaction was held at −50° C. to −45° C. for two hours. The reaction mixture was diluted with DCM (100 mL) and TEA (118.0 mL, 849 mmol) was added slowly via addition funnel. Additional DCM (150 mL) was added to aid in stirring and the temperature maintained at −25° C. for 30 minutes. The reaction was quenched by the addition of 1M NaHSO₄. The color of the mixture turned from clear to a medium/dark purple color over about 5 minutes, and then to a clear biphasic mixture over the next 20 min. The organic extracts were further washed with 2×1M NaHSO₄, NaHCO₃, and brine. The combined organic extracts were dried over MgSO₄, filtered, and concentrated under reduced pressure to provide a clear liquid (62.3 g).

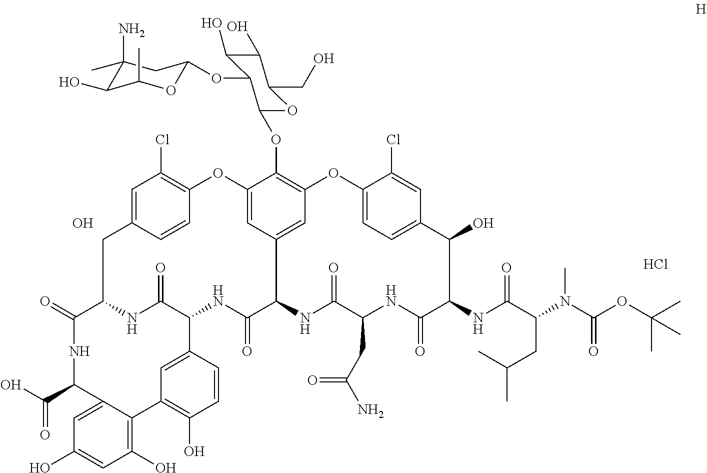

To a solution of vancomycin hydrochloride (10.00 g, 6.73 mmol) in dry DMSO (25 mL) under Argon at ambient temperature was added di-tert-butyl dicarbonate (1.91 g, 8.75 mmol). The reaction mixture was stirred at ambient temperature for 16 hrs. and added dropwise to dichloromethane (500 mL). The resulting slurry was filtered, washed with dichloromethane and dried under high vacuum to provide BOC-vancomycin HCl (13.48 g).

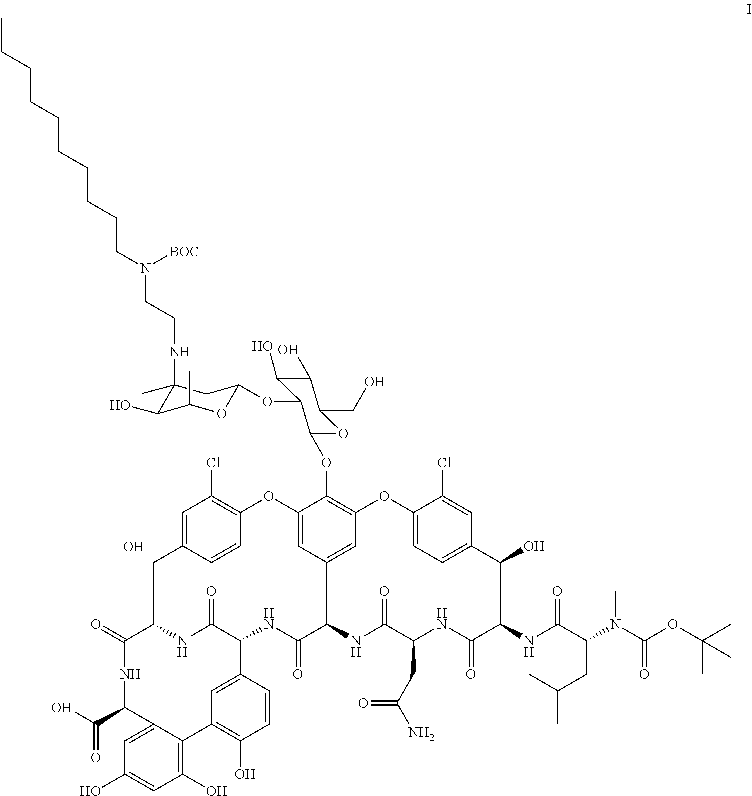

A mixture of BOC-vancomycin HCl (H, 1.500 g, 0.946 mmol), Intermediate G (425 mg, 1.419 mmol), sodium cyanoborohydride (238 mg, 3.78 mmol) and diisopropylethyl amine (330 mL, 1.89 mmol) in DMF (6 mL) and methanol (2 mL) was heated under Argon at 70 C for 16 hrs. The reaction was cooled to ambient temperature and added dropwise to a 1:1 mixture of acetone: diethyl ether (˜150 mL). The resulting slurry was filtered, washed with diethyl ether and dried under high vacuum to provide a white solid (1.67 g).

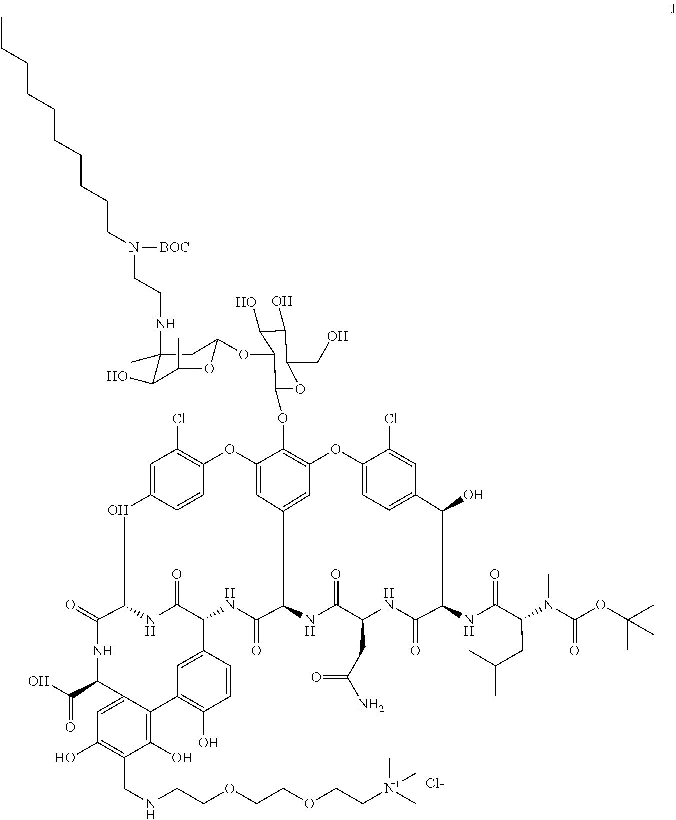 [SMILES: CCCCCCCCCCN(CCNC1(C)CC(OC2C(Oc3c4cc5cc3Oc3ccc(cc3Cl)[C@@H](O)[C@@H](NC(=O)[C@@H](CC(C)C)N(C)C(=O)OC(C)(C)C)C(=O)N[C@@H](CC(N)=O)C(=O)N[C@H]5C(=O)N[C@H]3C(=O)N[C@@H](Oc5ccc(c(Cl)c5)O4)C(=O)N[C@H](C(=O)O)c4cc(O)c(CNCCOCCOCC[N+](C)(C)C)c(O)c4-c4cc3ccc4O)OC(CO)C(O)C2O)OC(C)C1O)C(=O)OC(C)(C)C ; [Cl-]]

Intermediate I (505 mg, 0.276 mmol), diisopropylethyl amine (1.2 mL, 6.89 mmol) and Intermediate C (586 mg, 2.23 mmol) were dissolved in acetonitrile (3 mL) and water (2 mL). The mixture was cooled to 4 C and aqueous formaldehyde (204 L, 40.5 mg/mL, 0.276 mmol) was added. The reaction was stirred at 4 C for 16 hrs. and evaporated in vacuo. The residue was triturated with acetone, filtered and dried under high vacuum to provide an off-white solid (470 mg).

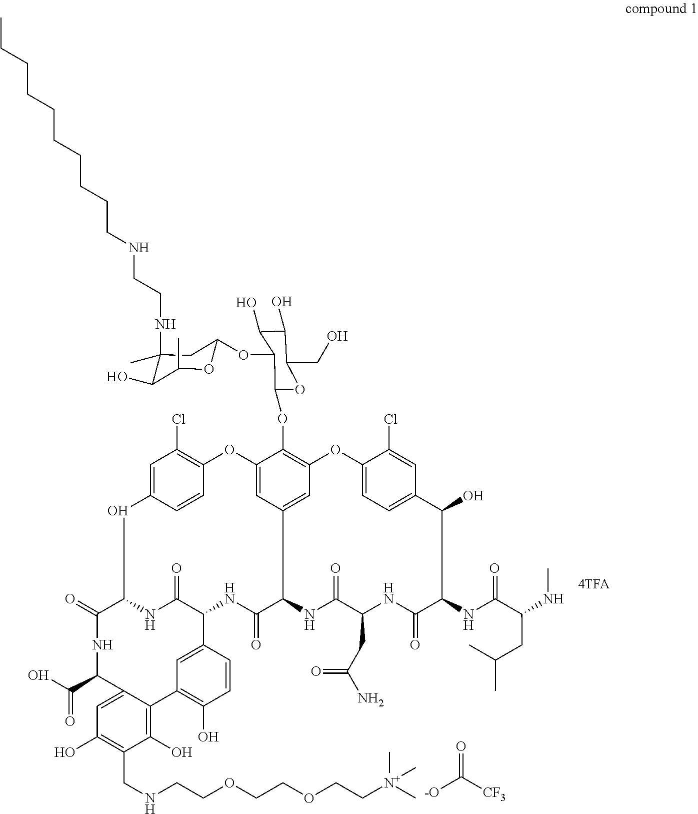

Intermediate J (450 mg, 0.217 mmol) was suspended in dichloromethane (4.0 mL) and cooled to 4 C. Trifluoroacetic acid (700 L, 9.4 mmol) was added and the reaction was stirred for approximately 1.5 hrs. Diethyl ether (˜15 mL) was added over several minutes and the resulting slurry was filtered and dried under high vacuum. The crude product was purified by RP-HPLC providing 164 mgs of white lyophilisate as a TFA salt.

### Example 3

### Compounds of Formulas (IIIb) and (IVb)

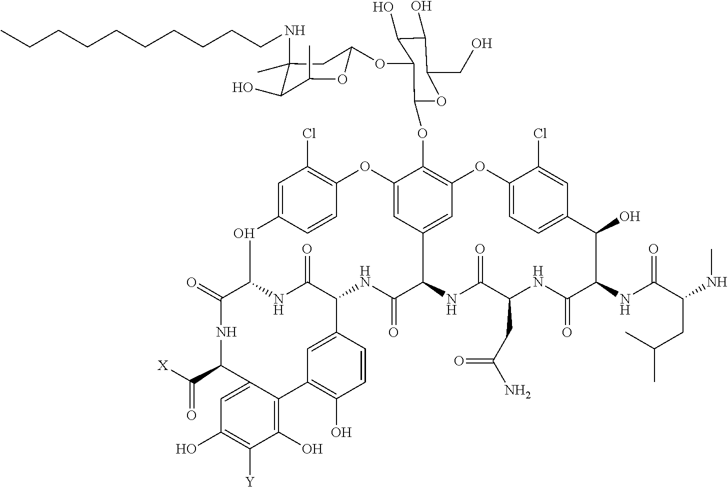

TABLE B

| Compound | X | Y |
|---|---|---|
| 4 | NHCH₂CH₂(OCH₂CH₂)₂NH₂ | H |
| 5 | OH | CH₂NHCH₂CH₂(OCH₂CH₂)₂NH₂ |
| 6 | OH | CH₂NHCH₂CH₂(OCH₂CH₂)₃N(CH₃)₃ |

### Example 4

### Compounds of Formulas (IIIc), (IVc), and (Vc)

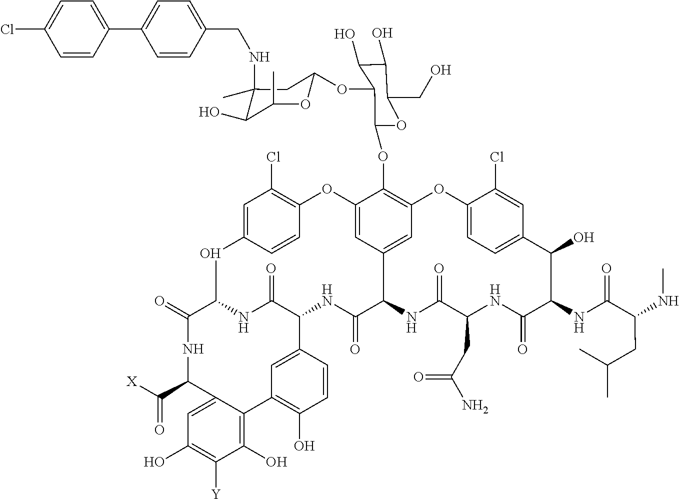

TABLE C

| Compound | X | Y |
|---|---|---|
| 7 | NHCH₂CH₂(OCH₂CH₂)₂N(CH₃)₂ | CH₂NHCH₂CH₂(OCH₂CH₂)₂N(CH₃)₂ |
| 8 | NHCH₂CH₂(OCH₂CH₂)₂N(CH₃)₃ | H |
| 9 | NHCH₂CH₂(OCH₂CH₂)₂NH₂ | H |
| 10 | NHCH₂CH₂(OCH₂CH₂)₂N(CH₃)₂ | H |
| 11 | OH | CH₂NHCH₂CH₂(OCH₂CH₂)₂NH₂ |
| 12 | OH | CH₂NHCH₂CH₂(OCH₂CH₂)₂N(CH₃)₂ |
| 13 | OH | CH₂NHCH₂CH₂(OCH₂CH₂)₂N(CH₃)₃ |
| 14 | OH | CH₂NHCH₂CH₂(OCH₂CH₂)₃N(CH₃)₂ |
| 15 | OH | CH₂NHCH₂CH₂(OCH₂CH₂)₃NH₂ |
| 16 | OH | CH₂NHCH₂CH₂(OCH₂CH₂)₃N(CH₃)₃ |
| 17 | NHCH₂CH₂(OCH₂CH₂)₃N(CH₃)₂ | H |
| 18 | NHCH₂CH₂(OCH₂CH₂)₃N(CH₃)₃ | H |
| 19 | NHCH₂CH₂(OCH₂CH₂)₃NH₂ | H |
| 20 | NHCH₂CH₂(OCH₂CH₂)₂N(CH₃)₃ | H |
| 21 | NHCH₂CH₂(OCH₂CH₂)₃N(CH₃)₃ | CH₂NHCH₂CH₂(OCH₂CH₂)₃N(CH₃)₃ |
| 22 | NHCH₂CH₂(OCH₂CH₂)₂NH₂ | CH₂NHCH₂CH₂(OCH₂CH₂)₂NH₂ |
| 23 | NHCH₂CH₂(OCH₂CH₂)₃N(CH₃)₂ | CH₂NHCH₂CH₂(OCH₂CH₂)₃N(CH₃)₂ |
| 24 | NHCH₂CH₂(OCH₂CH₂)₃NH₂ | CH₂NHCH₂CH₂(OCH₂CH₂)₃NH₂ |

### Example 5

### Compounds of Formula (IVd)

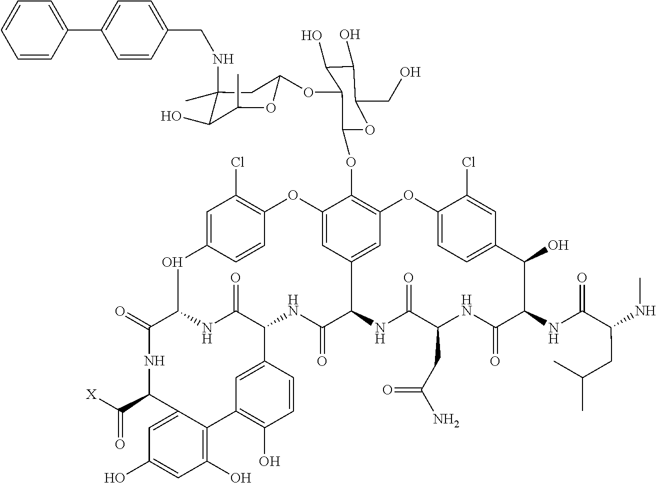

TABLE D

| Compound | X |
|---|---|
| 25 | NHCH₂CH₂(OCH₂CH₂)₂N(CH₃)₂ |
| 26 | NHCH₂CH₂(OCH₂CH₂)₂N(CH₃)₃ |

### Example 6

### Compounds of Formula (IIIe) and (IVe)

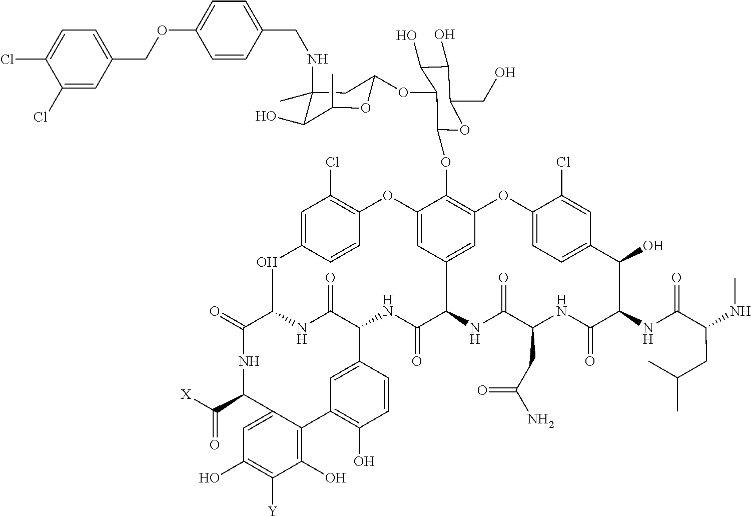

TABLE E

| Compound | X | Y |
|---|---|---|
| 27 | NHCH₂CH₂ | H |
| | (OCH₂CH₂)₂NH₂ | |
| 28 | OH | CH₂NHCH₂CH₂(OCH₂CH₂)₂NH₂ |
| 29 | OH | CH₂NHCH₂CH₂(OCH₂CH₂)₂N(CH₃)₃ |

### Example 7

### Compounds of Formula (IVf)

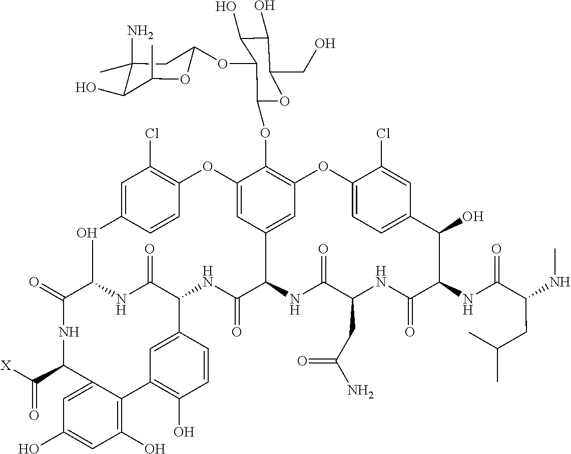

TABLE F

| Compound | X |
|---|---|
| 30 | NHCH₂CH₂(OCH₂CH₂)₂NH₂ |
| 31 | NHCH₂CH₂(OCH₂CH₂)₁₉NH₂ |

### Example 8

### Spectrum of Activity and Potency Against Gram-Positive Pathogens with Defined Resistance Phenotypes

Compounds of the invention were screened for antimicrobial activity against Gram-positive isolates having well defined and clinically relevant antimicrobial resistance phenotypes. Bacterial clinical isolates included in this investigation were (number tested): (i) Staphylococcus aureus (65 strains; 22 wildtype methicillin-susceptible (MSSA); 22 methicillin-resistant (MRSA); 5 vancomycin-intermediate (VISA); 10 heterogeneous VISA (hVISA); and 6 vancomycin-resistant (VRSA)), (ii) Staphylococcus epidermidis (43 strains; 21 wildtype methicillin-susceptible (MSCoNS); and 22 methicillin-resistant (MRCoNS)), (iii) Enterococcus faecium (41 strains; 21 wildtype strains, 10 VanA-type (vancomycin-resistant enterococci; VRE); and 10 VanB-type (VRE)), (iv) Enterococcus faecalis (46 strains; 23 wildtype strains; 11 VanA-type (VRE); and 12 VanB-type (VRE)), (v) vanC-carrying enterococci (22 strains; 11 Enterococcus casseliflavus, and 11 Enterococcus gallinarum), (vi) Streptococcus pneumoniae (22 strains; 11 wildtype strains; and 11 multidrug-resistant (MDR) strains), and (vii) β-hemolytic streptococci (23 strains; 11 Streptococcus pyogenes and 12 Streptococcus agalactiae). Resistance phenotypes were determined by reference broth microdilution tests followed by confirmational techniques as required or specified by Clinical and Laboratory Standards Institute (CLSI; M07-A8, 2009) criteria (Methods for dilution antimicrobial susceptibility tests for bacteria that grow aerobically. Document M07-A8. Wayne, Pa.: CLSI). VISA and VRSA strains were provided by the Network on Antimicrobial Resistance in S. aureus.

Antimicrobial Susceptibility Testing:

Solvent, diluents and dilution procedures utilized for all tested compounds followed the Clinical and Laboratory Standards Institute (CLSI) recommendations for water-insoluble agents (Performance standards for antimicrobial susceptibility testing, 20^{th} information supplement M100-520. Wayne, Pa.: CLSI; Table 7A; M100-S20-U, 2010). Stock solutions were prepared by dissolving each dry powder in glass container using DMSO (100%) to obtain a final concentration of 1,600 μg/mL. Stock solutions were serial diluted (1:2) in DMSO (100%) using glass macropipettes. A final dilution step (1:50) was performed using Mueller-Hinton broth (MHB) containing 0.004% of polysorbate (P-80). A total of 100 μL of final concentrations of test compounds containing P-80 (0.004%; final testing concentration, 0.002%) were dispensed in 96-well plates. MHB supplemented with 2-5% lysed horse blood was used for testing fastidious streptococci; MHB also contained P-80 (0.002%). Validation of the minimum inhibitory concentration (MIC) values obtained for test compounds and comparator compounds were performed by concurrent testing of CLSI-recommended (M100-S20-U, 2010) quality control (QC) American Type Culture Collection (ATCC) strains: S. aureus ATCC 29213, E. faecalis ATCC 29212 and S. pneumoniae ATCC 49619. Test compounds (0.008-16 μg/mL) and comparator agents (0.03-64 μg/mL) were tested to 12 log₂ dilution steps, except for linezolid (11 log₂ dilution steps; 0.03-32 μg/mL). Interpretation of MIC values were performed according to published CLSI (M100-S20-U, 2010) and European Committee on Antimicrobial Susceptibility Testing (EUCAST, 2010) breakpoints, when available. QC MIC results obtained for comparators were interpreted according to published criteria per CLSI M100-S20-U (2010).

Results:

Activity of test compounds tested against S. aureus and resistance subsets. Overall, the investigational compounds displayed MIC₅₀ results of 0.03 μg/mL (compound 3), 0.06 μg/mL (compounds 1, 2, 10, 27, 28, and 29) and 0.12 μg/mL (compounds 9, 11, 12, 13, and 14; Table 1). The most active test compounds (MIC₅₀, 0.03-0.06 μg/mL and MIC₉₀, 0.12 μg/mL) tested against S. aureus were four- to eight-fold more potent than daptomycin (MIC_{50/90}, 0.25/1 μg/mL), eight- to 64-fold more potent than teicoplanin (MIC_{50/90}, 0.5/8 μg/mL), 16- to 32-fold more potent than vancomycin (MIC_{50/90}, 1/4 μg/mL) and eight- to 32-fold more potent than linezolid (MIC_{50/90}, 1/1 μg/mL; Table 1). Each compound tested exhibited equivalent MIC₅₀ and modal MIC values when tested against MSSA and MRSA strains; except for compound 2, where MIC₅₀ and modal MIC values (0.03 μg/mL for both) against MSSA were two-fold lower compared with MRSA (0.06 μg/mL for both; Table 2). MIC₅₀ values for the test compounds gradually increased when tested against hVISA (MIC₅₀, 0.06-0.12 μg/mL), VISA (MIC₅₀, 0.12-0.5 μg/mL) and VRSA (MIC₅₀, 1-8 μg/mL; Table 3). Compounds 2 and 3 (MIC₅₀, 1 μg/mL, for both) showed the lowest MIC₅₀ results when tested against a rare collection of VRSA (Table 3).

Activity of test compounds tested against S. epidermidis. Overall, compound 27 (MIC_{50/90}, 0.03/0.03 μg/mL), compound 28 (MIC_{50/90}, 0.03/0.03 μg/mL) and compound 3 (MIC_{50/90}, 0.03/0.03 μg/mL) were equally active when tested against S. epidermidis (Table 4). Compounds 3, 27, and 28 (MIC_{50/90}, 0.03/0.03 μg/mL) were eight- to 16-fold more potent than daptomycin (MIC_{50/90}, 0.25/0.5 μg/mL), 16- to 32-fold more potent than linezolid (MIC_{50/90}, 0.5/1 μg/mL) and 32- to 128-fold more potent than vancomycin (MIC_{50/90}, 1/2 μg/mL) and teicoplanin (MIC_{50/90}, 2/4 μg/mL) tested against S. epidermidis (Table 4).

Activity of test compounds tested against E. faecalis. Compound 3 (MIC_{50/90}, 0.06/0.06 μg/mL) was the most active agent tested against vancomycin-susceptible E. faecalis strains, followed by compounds 1, 2, and 28 (all MIC_{50/90}, 0.06/0.12 μg/mL) and compounds 10, 27, and 29 (all MIC_{50/90}, 0.12/0.12 μg/mL; Table 5). When tested against VanB vancomycin-resistant E. faecalis (Table 6), investigational agents showed similar potencies (≦two-fold differences in the MIC₅₀ and MIC₉₀ results) compared with their respective susceptible counterpart (Table 5). The comparator agents, daptomycin (MIC₅₀, 0.5-1 μg/mL and MIC₉₀, 1-2 μg/mL) and linezolid (MIC_{50/90}, 1/1 μg/mL) showed similar activities when tested against E. faecalis, regardless of vancomycin susceptibility (Tables 5 and 6). Overall, all test compounds exhibited higher (16- to 128-fold) MIC₅₀ (2-8 μg/mL) and MIC₉₀ (2-16 μg/mL) results when tested against VanA-type E. faecalis compared with wildtype strains (Tables 5 and 6). Among the test compounds, compound 27 (MIC_{50/90}, 2/2 μg/mL) was the least affected (16-fold increase when compared with susceptible strains) agent when tested against VanA-type E. faecalis and inhibited all strains at ≦2 μg/mL (Table 6). Compound 27 (MIC_{50/90}, 2/2 μg/mL) tested against VanA vancomycin-resistant E. faecalis demonstrated similar MIC_{50/90} results compared with linezolid (MIC_{50/90}, 1/1 μg/mL) and daptomycin (MIC_{50/90}, 1/2 μg/mL; Table 6).

Activity of compounds tested against E. faecium. Compounds 2 and 3 (MIC_{50/90}, 0.015/0.03 μg/mL, for both) were the most active agents tested against vancomycin-susceptible E. faecium, followed by compounds 1, 27, 28, and 29 (all MIC_{50/90}, 0.03/0.06 μg/mL; Table 7). These test compounds were 16- to 32-fold more active than vancomycin (MIC_{50/90}, 0.5/1 μg/mL; Table 7) when tested against vancomycin-susceptible E. faecium. MIC_{50/90} result comparisons demonstrated that each agent displayed comparable potencies (≦two-fold differences in the MIC₅₀ and MIC₉₀ results) when tested against vancomycin-susceptible and -resistant (VanB) E. faecium (Tables 7 and 9). Among the investigational compounds tested against VanA-type E. faecium, compound 27 (MIC_{50/90}, 0.5/1 μg/mL) and compound 28 (MIC_{50/90}, 0.5/1 μg/mL) were the most active (Table 8). In addition, these agents were up to four-fold more potent than daptomycin (MIC_{50/90}, 2/2 μg/mL) and linezolid (MIC_{50/90}, 1/1 μg/mL).

Activity of compounds tested against enterococcal strains intrinsically harboring the vanC gene (Table 10). E. casseliflavus and E. gallinarum were very susceptible to compounds 2 and 3 (MIC_{50/90}, 0.06/0.12 μg/mL, for both), and compounds 1, 27 and 28 (all MIC_{50/90}, 0.06/0.25 μg/mL). The compounds above described were eight- to 16-fold more potent than the comparator agents teicoplanin, daptomycin and linezolid (all MIC_{50/90}, 1/2 μg/mL) and 32- to 64-fold more active than vancomycin (MIC_{50/90}, 4/4 μg/mL).

Activity of compounds tested against β-hemolytic streptococci and S. pneumoniae (Table 11). When tested against β-hemolytic streptococci, compounds 9, 11, 12, 13, 14, and 27 (all MIC₅₀, 0.06 μg/mL) were two-fold less active than compounds 1, 2, 3, 10, 28, and 29 (all MIC₅₀, 0.03 μg/mL). Compounds 1, 2, 3, 10, 28, and 29 (all MIC₅₀, 0.03 μg/mL and MIC₉₀, 0.06-0.12 μg/mL) were four- to eight-fold more potent than vancomycin (MIC_{50/90}, 0.25/0.5 μg/mL) and two- to four-fold more potent than teicoplanin (MIC_{50/90}, 0.12/0.25 μg/mL) and daptomycin (MIC_{50/90}, 0.12/0.25 μg/mL). When tested against S. pneumoniae strains, the compounds 1, 2, and 3 exhibited the lowest MIC_{50/90} results (all 0.015/0.03 μg/mL), followed by compounds 10, 27, 28 and 29 (all MIC_{50/90}, 0.03/0.06 μg/mL). Compounds 1, 2, and 3 (MIC_{50/90}, 0.015/0.03 μg/mL) were four- to 16-fold more active than vancomycin (MIC_{50/90}, 0.25/0.5 μg/mL), teicoplanin (MIC_{50/90}, 0.12/0.12 μg/mL) and daptomycin (MIC_{50/90}, 0.12/0.25 μg/mL), and 32-fold more potent than and linezolid (MIC_{50/90}, 0.5/1 μg/mL).

Summary of Results:

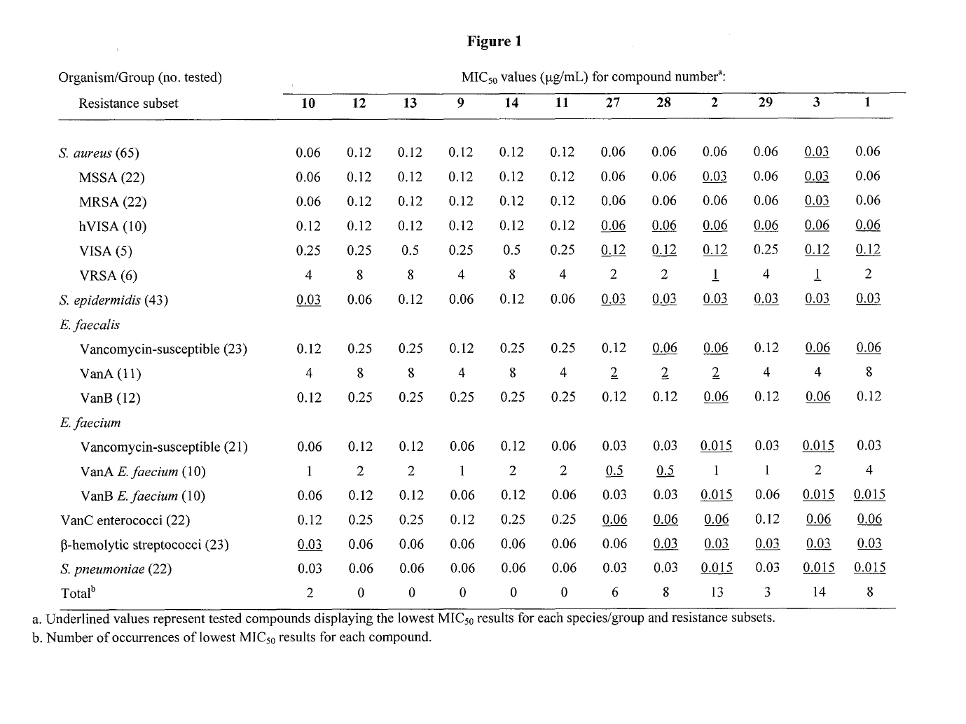

Overall, compound 3 exhibited the lowest MIC₅₀ results when tested against staphylococcal strains and respective resistance subsets (Tables 1, 2, 3, 4 and FIG. 1). In addition, compounds 2 and 3 were the most active (MIC₅₀ results) compounds tested against VRSA (Table 3 and FIG. 1).

When tested against vancomycin-susceptible E. faecium, test compounds were two- to four-fold more potent compared with vancomycin-susceptible E. faecalis strains (Tables 5, 7 and FIG. 1).

In general, test compounds demonstrated comparable MIC results when tested against the vancomycin-susceptible and VanB vancomycin-resistant enterococcal species (Tables 5, 6, 7, 9 and FIG. 1). However, these agents were less active against VanA vancomycin-resistant enterococci compared with their respective susceptible counterparts.

Compound 27 (MIC_{50/90}, 2/2 μg/mL), compound 28 (MIC_{50/90}, 2/4 μg/mL) and compound 2 (MIC_{50/90}, 2/4 μg/mL) were the most active agents tested against VanA vancomycin-resistant E. faecalis, while compounds 27 and 28 (MIC_{50/90}, 0.5/1 μg/mL, for both) were the most potent tested against VanA vancomycin-resistant E. faecium (Tables 6, 8 and FIG. 1).

Enterococcal species carrying the intrinsic vanC gene were very susceptible to several compounds (MIC₅₀, 0.06 μg/mL and MIC₉₀, 0.12-0.25 μg/mL) and inhibited all strains at ≦0.25 μg/mL, except for compound 29 (Table 10 and FIG. 1).

When tested against β-hemolytic streptococci, compounds 1, 2, 3, 10, 28, and 29 (MIC₅₀, 0.03 μg/mL and MIC₉₀, 0.06-0.12 μg/mL) demonstrated the lowest MIC results, whereas compounds 1, 2 and 3 (MIC_{50/90}, 0.015/0.03 μg/mL) were the most potent against S. pneumoniae (Table 11 and FIG. 1). Comparison of MIC₅₀ results demonstrated that compounds 2 and 3 exhibited the overall highest potency when tested against this collection of Gram-positive organisms (FIG. 1). While compound 3 appears to be slightly more active against MRSA strains, compound 2 seems to be more potent against VanA vancomycin-resistant strains

TABLE 1

| Organism | | |
|---|---|---|
| (no. tested) | MIC (μg/mL) | |
| Compound | 50% | 90% |
| S. aureus (65) | | |
| 1 | 0.06 | 0.12 |
| 2 | 0.06 | 0.12 |
| 3 | 0.03 | 0.12 |
| 9 | 0.12 | 0.5 |
| 10 | 0.06 | 0.25 |
| 11 | 0.12 | 0.5 |
| 12 | 0.12 | 0.5 |
| 13 | 0.12 | 0.5 |
| 14 | 0.12 | 0.5 |
| 27 | 0.06 | 0.25 |
| 28 | 0.06 | 0.12 |
| 29 | 0.06 | 0.25 |
| Vancomycin | 1 | 4 |
| Teicoplanin | 0.5 | 8 |
| Daptomycin | 0.25 | 1 |
| Linezolid | 1 | 1 |

TABLE 2

| Organism | | |
|---|---|---|
| (no. tested) | MIC (μg/mL) | |
| Compound | 50% | 90% |
| MSSA (22) | | |
| 1 | 0.06 | 0.06 |
| 2 | 0.03 | 0.12 |
| 3 | 0.03 | 0.06 |
| 9 | 0.12 | 0.12 |
| 10 | 0.06 | 0.06 |
| 11 | 0.12 | 0.12 |
| 12 | 0.12 | 0.12 |
| 13 | 0.12 | 0.25 |
| 14 | 0.12 | 0.25 |
| 27 | 0.06 | 0.06 |
| 28 | 0.06 | 0.06 |
| 29 | 0.06 | 0.12 |
| Vancomycin | 0.5 | 1 |
| Teicoplanin | 0.5 | 1 |
| Daptomycin | 0.25 | 0.5 |
| Linezolid | 1 | 1 |
| MRSA (22) | | |
| 2 | 0.06 | 0.12 |
| 3 | 0.03 | 0.06 |
| 9 | 0.12 | 0.12 |
| 10 | 0.06 | 0.12 |
| 11 | 0.12 | 0.12 |
| 12 | 0.12 | 0.12 |
| 13 | 0.12 | 0.12 |
| 14 | 0.12 | 0.12 |
| 27 | 0.06 | 0.06 |
| 28 | 0.06 | 0.06 |
| 29 | 0.06 | 0.12 |
| VA078 | 0.06 | 0.06 |
| Vancomycin | 0.5 | 1 |
| Teicoplanin | 0.5 | 0.5 |
| Daptomycin | 0.25 | 0.5 |
| Linezolid | 1 | 1 |

TABLE 3

| Organism | | |
|---|---|---|
| (no. tested) | MIC (μg/mL) | |
| Compound | 50% | 90% |
| hVISA (10) | | |
| 1 | 0.06 | 0.06 |
| 2 | 0.06 | 0.12 |
| 3 | 0.06 | 0.06 |
| 9 | 0.12 | 0.12 |
| 10 | 0.12 | 0.12 |
| 11 | 0.12 | 0.12 |
| 12 | 0.12 | 0.12 |
| 13 | 0.12 | 0.12 |
| 14 | 0.12 | 0.12 |
| 27 | 0.06 | 0.06 |
| 28 | 0.06 | 0.06 |
| 29 | 0.06 | 0.12 |
| VISA (5) | | |
| 1 | 0.12 | — |
| 2 | 0.12 | — |
| 3 | 0.12 | — |
| 9 | 0.25 | — |
| 10 | 0.25 | — |
| 11 | 0.25 | — |
| 12 | 0.25 | — |
| 13 | 0.5 | — |
| 14 | 0.5 | — |
| 27 | 0.12 | — |
| 28 | 0.12 | — |
| 29 | 0.25 | — |
| VRSA (6) | | |
| 1 | 2 | — |
| 2 | 1 | — |
| 3 | 1 | — |
| 9 | 4 | — |
| 10 | 4 | — |
| 11 | 4 | — |
| 12 | 8 | — |
| 13 | 8 | — |
| 14 | 8 | — |
| 27 | 2 | — |
| 28 | 2 | — |
| 29 | 4 | — |

TABLE 4

| Organism | MIC | |
|---|---|---|
| (no. tested) | (μg/mL) | |
| Compound | 50% | 90% |
| S. epidermidis (43) | | |
| 1 | 0.03 | 0.06 |
| 2 | 0.03 | 0.06 |
| 3 | 0.03 | 0.03 |
| 9 | 0.06 | 0.06 |
| 10 | 0.03 | 0.06 |
| 11 | 0.06 | 0.06 |
| 12 | 0.06 | 0.12 |
| 13 | 0.12 | 0.12 |
| 14 | 0.12 | 0.12 |
| 27 | 0.03 | 0.03 |
| 28 | 0.03 | 0.03 |
| 29 | 0.03 | 0.06 |
| Vancomycin | 1 | 2 |
| Teicoplanin | 2 | 4 |
| Daptomycin | 0.25 | 0.5 |
| Linezolid | 0.5 | 1 |

TABLE 5

| Organism (no. tested) | MIC (μg/mL) | |
|---|---|---|
| Compound | 50% | 90% |
| Vancomycin-susceptible (23) | | |
| 1 | 0.06 | 0.12 |
| 2 | 0.06 | 0.12 |
| 3 | 0.06 | 0.06 |
| 9 | 0.12 | 0.25 |
| 10 | 0.12 | 0.12 |
| 11 | 0.25 | 0.25 |
| 12 | 0.25 | 0.25 |
| 13 | 0.25 | 0.25 |
| 14 | 0.25 | 0.25 |
| 27 | 0.12 | 0.12 |
| 28 | 0.06 | 0.12 |
| 29 | 0.12 | 0.12 |
| Vancomycin | 1 | 2 |
| Teicoplanin | 0.5 | 0.5 |
| Daptomycin | 1 | 2 |
| Linezolid | 1 | 1 |

TABLE 6

| Organism | MIC | |
|---|---|---|
| (no. tested) | (μg/mL) | |
| Compound | 50% | 90% |
| VanA (11) | | |
| 1 | 8 | 8 |
| 2 | 2 | 4 |
| 3 | 4 | 8 |
| 9 | 4 | 8 |
| 10 | 4 | 8 |
| 11 | 4 | 8 |
| 12 | 8 | 16 |
| 13 | 8 | 16 |
| 14 | 8 | 16 |
| 27 | 2 | 2 |
| 28 | 2 | 4 |
| 29 | 4 | 4 |
| Vancomycin | >64 | >64 |
| Teicoplanin | 64 | >64 |
| Daptomycin | 1 | 2 |
| Linezolid | 1 | 1 |
| VanB (12) | | |
| 1 | 0.12 | 0.12 |
| 2 | 0.06 | 0.12 |
| 3 | 0.06 | 0.12 |
| 9 | 0.25 | 0.25 |
| 10 | 0.12 | 0.25 |
| 11 | 0.25 | 0.25 |
| 12 | 0.25 | 0.25 |
| 13 | 0.25 | 0.5 |
| 14 | 0.25 | 0.25 |
| 27 | 0.12 | 0.12 |
| 28 | 0.12 | 0.12 |
| 29 | 0.12 | 0.12 |
| Vancomycin | >64 | >64 |
| Teicoplanin | 0.5 | 1 |
| Daptomycin | 0.5 | 1 |
| Linezolid | 1 | 1 |

TABLE 7

| Organism (no. tested) | MIC (μg/mL) | |
|---|---|---|
| Compound | 50% | 90% |
| Vancomycin-susceptible (21) | | |
| 1 | 0.03 | 0.03 |
| 2 | 0.015 | 0.03 |
| 3 | 0.015 | 0.03 |
| 9 | 0.06 | 0.12 |
| 10 | 0.06 | 0.06 |
| 11 | 0.06 | 0.12 |
| 12 | 0.12 | 0.12 |
| 13 | 0.12 | 0.12 |
| 14 | 0.12 | 0.12 |
| 27 | 0.03 | 0.06 |
| 28 | 0.03 | 0.06 |
| 29 | 0.03 | 0.06 |
| Vancomycin | 0.5 | 1 |
| Teicoplanin | 1 | 1 |
| Daptomycin | 2 | 2 |
| Linezolid | 1 | 2 |

TABLE 8

| Organism | MIC | |
|---|---|---|
| (no. tested) | (μg/mL) | |
| Compound | 50% | 90% |
| VanA (10) | | |
| 1 | 4 | 8 |
| 2 | 1 | 2 |
| 3 | 2 | 4 |
| 9 | 1 | 2 |
| 10 | 1 | 2 |
| 11 | 2 | 4 |
| 12 | 2 | 4 |
| 13 | 2 | 4 |
| 14 | 2 | 4 |
| 27 | 0.5 | 1 |
| 28 | 0.5 | 1 |
| 29 | 1 | 2 |
| Vancomycin | >64 | >64 |
| Teicoplanin | 64 | >64 |
| Daptomycin | 2 | 2 |
| Linezolid | 1 | 1 |

TABLE 9

| Organism | MIC | |
|---|---|---|
| (no. tested) | (μg/mL) | |
| Compound | 50% | 90% |
| VanB (10) | | |
| 1 | 0.015 | 0.03 |
| 2 | 0.015 | 0.03 |
| 3 | 0.015 | 0.015 |
| 9 | 0.06 | 0.12 |
| 10 | 0.06 | 0.06 |
| 11 | 0.06 | 0.12 |
| 12 | 0.12 | 0.12 |
| 13 | 0.12 | 0.12 |
| 14 | 0.12 | 0.12 |
| 27 | 0.03 | 0.03 |
| 28 | 0.03 | 0.06 |
| 29 | 0.06 | 0.06 |
| Vancomycin | 64 | >64 |
| Teicoplanin | 1 | 1 |
| Daptomycin | 2 | 2 |
| Linezolid | 1 | 1 |

TABLE 10

| Organism | MIC | |
|---|---|---|
| (no. tested) | (μg/mL) | |
| Compound | 50% | 90% |
| VanC enterococci (22) | | |
| 1 | 0.06 | 0.25 |
| 2 | 0.06 | 0.12 |
| 3 | 0.06 | 0.12 |
| 9 | 0.12 | 0.5 |
| 10 | 0.12 | 0.25 |
| 11 | 0.25 | 0.5 |
| 12 | 0.25 | 0.5 |
| 13 | 0.25 | 1 |
| 14 | 0.25 | 0.5 |
| 27 | 0.06 | 0.25 |
| 28 | 0.06 | 0.25 |
| 29 | 0.12 | 0.5 |
| Vancomycin | 4 | 4 |
| Teicoplanin | 1 | 2 |
| Daptomycin | 1 | 2 |
| Linezolid | 1 | 2 |

TABLE 11

| Organism (no. tested) | MIC (μg/mL) | |
|---|---|---|
| Compound | 50% | 90% |
| β-hemolytic streptococci (23) | | |
| 1 | 0.03 | 0.06 |
| 2 | 0.03 | 0.12 |
| 3 | 0.03 | 0.12 |
| 9 | 0.06 | 0.12 |
| 10 | 0.03 | 0.12 |
| 11 | 0.06 | 0.12 |
| 12 | 0.06 | 0.06 |
| 13 | 0.06 | 0.06 |
| 14 | 0.06 | 0.06 |
| 27 | 0.06 | 0.12 |
| 28 | 0.03 | 0.06 |
| 29 | 0.03 | 0.12 |
| Vancomycin | 0.25 | 0.5 |
| Teicoplanin | 0.12 | 0.25 |
| Daptomycin | 0.12 | 0.25 |
| Linezolid | 1 | 1 |
| S. pneumoniae (22) | | |
| 1 | 0.015 | 0.03 |
| 2 | 0.015 | 0.03 |
| 3 | 0.015 | 0.03 |
| 9 | 0.06 | 0.06 |
| 10 | 0.03 | 0.06 |
| 11 | 0.06 | 0.06 |
| 12 | 0.06 | 0.06 |
| 13 | 0.06 | 0.12 |
| 14 | 0.06 | 0.12 |
| 27 | 0.03 | 0.06 |
| 28 | 0.03 | 0.06 |
| 29 | 0.03 | 0.06 |
| Vancomycin | 0.25 | 0.5 |
| Teicoplanin | 0.12 | 0.12 |
| Daptomycin | 0.12 | 0.25 |
| Linezolid | 0.5 | 1 |

### Example 9

### Comparative In Vivo Efficacy Against S. aureus in the Neutropenic Murine Thigh Infection Model

As described above, test compounds demonstrate in vitro activity against gram positive bacteria, including methicillin-resistance S. aureus. We used the neutropenic murine thigh infection model to determine and compare the in vivo activity of four compounds from this series against S. aureus. The viable burden of organisms in the thighs of treated and control animals were measured before and at several time points after antimicrobial administration. Three escalating intraperitoneal dose levels of the test compound doses included 1, 4, and 16 mg/kg. Mice had 10^{6.63} cfu/thigh of S. aureus ATCC 25923 in mice prior to the administration of drug treatment, respectively. The organism burden increased 10^{2.57} cfu/thighs in untreated control mice.

Methods:

Bacteria, media, and antibiotic. A strain of S. aureus ATCC 25923 was used. The organism was grown, subcultured, and quantified in Mueller-Hinton broth (Difco Laboratories, Detroit, Mich.) and Mueller-Hinton agar (Difco Laboratories, Detroit, Mich.). Compounds 1, 2, 3, 9, 10, 11, 12, 13, 14, 27, 28, and 29 were tested.

Murine infection model. The neutropenic mouse thigh infection model has been used extensively for determination of pharmacokinetic/pharmacodynamic indice determination and prediction of antibiotic efficacy in patients Animals were maintained in accordance with the American Association for Accreditation of Laboratory Animal Care criteria. Six-week-old, specific-pathogen-free, female ICR/Swiss mice weighing 23 to 27 g were used for all studies (Harlan Sprague-Dawley, Indianapolis, Ind.). Mice were rendered neutropenic (neutrophils, <100/mm³) by injecting them with cyclophosphamide (Mead Johnson Pharmaceuticals, Evansville, Ind.) intraperitoneally 4 days (150 mg/kg) and 1 day (100 mg/kg) before thigh infection. Previous studies have shown that this regimen produces neutropenia in this model for 5 days. Broth cultures of freshly plated bacteria were grown to logarithmic phase overnight to an absorbance at 580 nm of 0.3 (Spectronic 88; Bausch and Lomb, Rochester, N.Y.). After a 1:10 dilution into fresh Mueller-Hinton broth, bacterial counts of the inoculum were 10^{7.19±0.50} CFU/ml for S. aureus. Thigh infections with each of the isolates were produced by injection of 0.1 ml of inoculum into the thighs of isoflurane-anesthetized mice 2 h before therapy.

Treatment protocol. Groups of two mice per dose and time point were infected with S. aureus in each thigh. Two hours after infection, neutropenic mice were treated with single intraperitoneal doses of 1, 4, and 16, mg/kg of each compound. An untreated control group of mice was used for each study. Groups of two mice per time point were euthanized at the start of therapy and 3, 6, 8, 12, and 24 h after therapy. The thighs were removed from these mice and processed immediately for CFU determination (four data points per dose-time point).

Data Analysis:

Area under the time kill curve was calculated for each treatment group and the untreated controls. The AUC from each treatment group was subtracted from the AUC from the untreated controls to estimate in vivo efficacy over the entire study period. The AUCc-t was compared among compounds.

Results:

In vivo time kill study. At the start of therapy, mice had 10^{6.63} cfu/thigh of S. aureus. The organism burden increased 10^{2.57} cfu/thigh of S. aureus in untreated control mice. Table 12 shows the maximal organism reduction for each compound compared to the burden at the start of therapy. The table also reports the entire time course efficacy compared to untreated control mice. The time course activity is estimated by calculating the area under the time kill curve using the trapezoidal rule for treated and untreated mice. The AUC in for each dose is subtracted from the AUC for untreated mice. The larger the AUC difference represents greater in vivo efficacy over time.

TABLE 12

| | 16 mg/kg | | 4 mg/kg | | 1 mg/kg | |
|---|---|---|---|---|---|---|
| Compound | AUCc-t* | Max Kill** | AUCc-t* | Max Kill** | AUCc-t* | Max Kill** |
| 1 | 60.4 | −1.07 | 32.7 | −1.14 | 28 | 0.15 |
| 2 | 55 | −1.13 | 40.1 | −1.01 | 29 | −0.28 |
| 3 | 52.9 | −1.08 | 32.3 | −0.41 | 28.4 | −0.2 |
| 9 | 24.6 | −0.01 | 17.2 | 0.5 | 7.4 | 0.98 |
| 10 | 25.6 | −0.02 | 22.4 | −0.06 | 10.7 | 0.43 |
| 11 | 34.7 | −0.39 | 10.8 | 0.46 | 12.9 | 0.59 |
| 12 | 29.3 | 0.0005 | 17.6 | −0.03 | 6.1 | 1.1 |
| 13 | 29.5 | 0.02 | 20.6 | 0.28 | 12.6 | 0.9 |
| 14 | 36.1 | 0.03 | 19.4 | 0.19 | 11.4 | 0.83 |
| 27 | 36.9 | −0.35 | 18.8 | −0.008 | 19.8 | 0.45 |
| 28 | 38.2 | −0.02 | 17.3 | 0.75 | 19.1 | 0.61 |
| 29 | 45.2 | −1.05 | 22.6 | 0.07 | 17.7 | 0.08 |

*AUC difference (Log₁₀ CFU/thigh)/hr between uninfected control and treated animals
**Maximum decrease (Log₁₀ CFU/thigh) from initial infection level

Many of the test compounds produced a reduction in organism burden in thighs compared to that at the start of therapy at the highest dose level examined. Four of the compounds (1, 2, 3, and 29) produced more than a 1 log₁₀ reduction in burden at this dose level. Therapy with two compounds (2 and 3) resulted in an organism reduction over the entire dose range. For the majority of compounds and doses, maximal activity was observed at the 6 hour time point. The area under the time kill curve was calculated for each treatment and control group. The largest AUC values (representing efficacy over the entire study period) were observed for compounds 1, 2, 3, and 29).

Conclusions:

Each of the compounds demonstrated in vivo efficacy against S. aureus in this neutropenic soft tissue infection model. Several of the compounds produced bactericidal characteristics and prolonged in vivo activity (1, 2, 3, and 29). Gross toxicity was not observed with any of the compounds over the dose range studied.

### Example 10

### Susceptibility Testing of Test Compounds, Vancomycin, and Linezolid Versus a Variety of Gram-Positive Bacteria

Organisms:

The test organisms were originally received from either the American Type Culture Collection (ATCC) or from clinical sources. Upon receipt, the isolates were streaked onto Trypticase soy agar (TSA) or TSA+5% sheep blood for streptococci. Colonies were harvested from these plates and a cell suspension was prepared in appropriate broth medium containing cryoprotectant. Aliquots were then frozen at −80° C. Prior to assay, the frozen seeds of the organisms were thawed and streaked for isolation onto TSA or TSA+5% sheep blood agar plates and incubated overnight at 35° C.

Test Media:

The medium employed for the MIC assay for most of the organisms was Mueller Hinton II Broth, prepared at 105% to offset the presence of 5% drug in the final test plate. Streptococcus isolates were tested in MHB II supplemented with 2% lysed horse blood (Cleveland Scientific H13913). The above media were used without further supplements for testing S. aureus ATCC 29213 (MMX100), and S. pneumoniae ATCC 49619 (MMX 1195), to determine whether the MIC values for vancomycin and linezolid in the assay were within CLSI quality control guidelines. Each of the assay organisms was tested in Tween 80-supplemented medium appropriate to the organism and also in Tween 80-supplemented medium plus 50% human serum. A stock solution of Tween 80 (Sigma P5188, Lot 025K005715) was prepared at 2% and autoclaved. The media for all the assay organisms were supplemented with Tween 80 at 0.002%.

Test Procedure:

The MIC assay method followed the procedure described by the Clinical and Laboratory Standards Institute and employed automated liquid handlers to conduct serial dilutions and liquid transfers. One-half volume of DMSO was added to each of the compounds (1, 2, 3, 23, 24, and 29), vancomycin, and linezolid and the solutions, followed by adding the other half volume as sterile deionized water (final DMSO concentration was 50% for the stock solutions). Stock concentrations of all test compounds were prepared at 640 μg/mL, which yielded a test concentration range of 16-0.015 μg/mL. The drug solutions were then serially-diluted in ‘mother plates’ on the Biomek 2000 (Beckman Coulter, Fullerton, Calif.). DMSO was the diluent in the mother plates. Using the Multimek 96 (Beckman Coulter, Fullerton, Calif.), 5 μL was transferred from each well of a mother plate into the corresponding well of a ‘daughter plate’, 96-well microplates containing 85 μL of one of the media described previously. From the overnight agar cultures of the isolates, standardized cell suspensions of each organism were prepared and diluted 1:19 in organism-appropriate medium. These diluted suspensions were used to inoculate the daughter plates using the Biomek 2000, 10 μL per well. Plates were stacked three high, covered with a lid, and bagged. Incubation was at 35° C. for 19 hours for Staphylococcus and Bacillus anthracis, and 20 hours for Streptococcus pneumoniae. Following incubation, the microplates were removed from the incubator and viewed from the bottom using a ScienceWare plate reader. A solubility control plate was observed for evidence of drug precipitation. The MIC was read and recorded as the lowest concentration of drug that inhibited visible growth of the organism.

Results:

No precipitation was observed in any of the uninoculated solubility control plates. Activity against B. anthracis was overall greater than that observed for S. aureus, while S. pneumoniae was the most sensitive organism tested. The following MICs (μg/mL) were observed against B. anthracis Sterne 105: compound 23 (0.12), compound 24 (0.03), compound 2 (0.03), compound 29 (0.03), compound 3 (≦0.015), and compound 1 (≦0.015).

### OTHER EMBODIMENTS

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

This application claims benefit of the U.S. Provisional Application Ser. No. 61/467,082, filed Mar. 24, 2011, which is incorporated herein by reference.

Other embodiments are within the claims.

What is claimed is:

1. A compound of formula (I), or a salt or prodrug thereof: 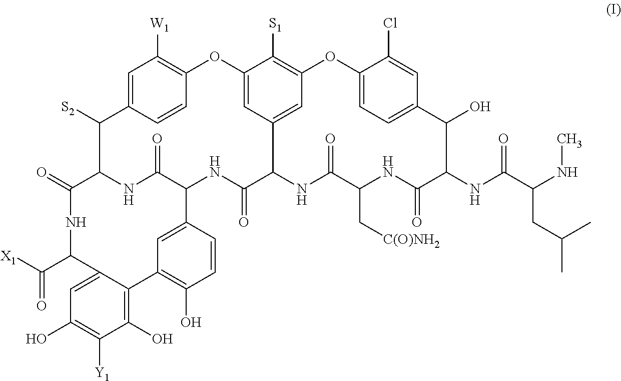
wherein,
W₁ is H or Cl;
X₁ is selected from N(R^{A})(CH₂CH₂O)ₐCH₂CH₂Z₁, OH, NH₂, NHR^{A1}, NR^{A1}R^{A2}, and OR^{A1};
Y₁ is selected from CH₂N(R^{B})(CH₂CH₂O)_{b}CH₂CH₂Z₂, H, CH₂NH₂, CH₂NHCOR^{B1}, CH₂NHCONHR^{B1}, CH₂NHCONR^{B1}R^{B2}, CH₂NHC(O)OR^{B1}, CH₂NHR^{B1}, CH₂NR^{B1}R^{B2}; CH₂NHSO₂R^{B1}, CH₂NHSO₂NHR^{B1}, CH₂NHSO₂NR^{B1}R^{B2}, and CH₂NHCH₂PO(OH)₂;
S₁ is a saccharide group selected from: 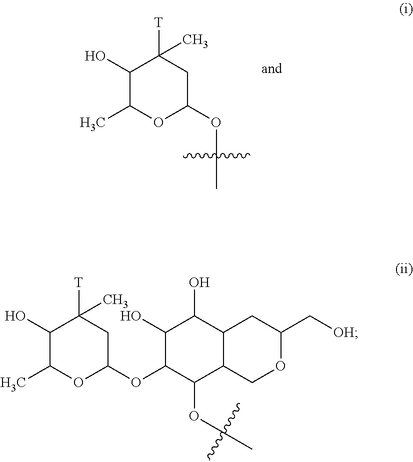
T is selected from —NH₂, —NH(CH₂)_{c}NHR^{T1}, —NHCO(CH₂)_{c}NHR^{T1}, —NHR^{T1}, —NH(CH₂)_{c}R^{T1}, and —NHCH₂—(C₆H₄), —O—R^{T1};
S₂ is OH or 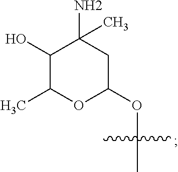 [SMILES: *OC1CC(C)(N)C(O)C(C)O1]
a is an integer from 1 to 20;
b is an integer from 1 to 20;
c is an integer from 1 to 3;
each of R^{A} and R^{B} is, independently, selected from H and C₁₋₄ alkyl;
each of R^{A1} and R^{A2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl;
each of R^{B1} and R^{B2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl;
R^{T1} is selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl;
each of Z₁ and Z₂ is, independently, selected from NH₂, NHR^{C1}, NR^{C1}R^{C2}, and NR^{C1}R^{C2}R^{C3};
each of R^{C1}, R^{C2}, and R^{C3} is, independently, selected from C₁₋₄ alkyl, C₂₋₄ alkenyl, and C₂₋₄ alkynyl,
provided that either X₁ is N(R^{A})(CH₂CH₂O)ₐCH₂CH₂Z₁ or Y₁ is CH₂N(R^{B})(CH₂CH₂O)_{b}CH₂CH₂Z₂.

2. The compound of claim 1, wherein T is selected from —NH₂, —NH(CH₂)₉CH₃, —NHCH₂CH₂NH(CH₂)₉CH₃, p-(p-chlorophenyl)benzyl-NH—, 4-phenylbenzyl-NH—, and 4-[(3,4-dichlorophenyl)methoxy]benzyl-NH—.

3. The compound of claim 1, wherein Z₁ or Z₂ is a quaternary amine.

4. The compound of claim 1, wherein Z₁ or Z₂ is NH₂, —N(CH₃)₂, or —N(CH₃)₃.

5. The compound of claim 1, wherein said compound is further described by formula (II), or a salt or prodrug thereof: 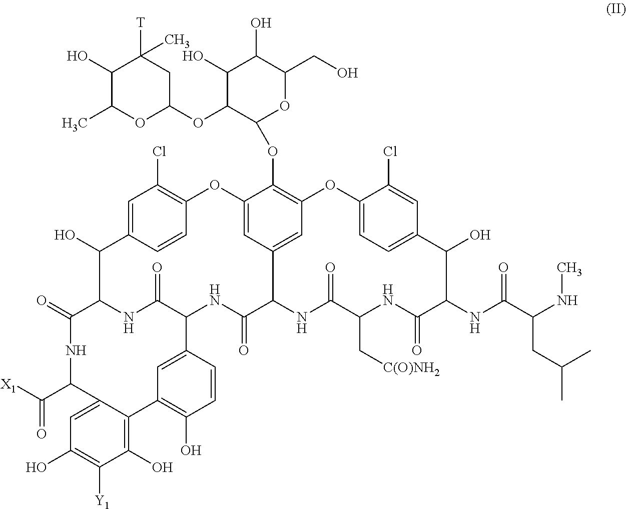
wherein X₁, Y₁, and T are as defined in formula (I).

6. The compound of claim 5, wherein T is —NH₂, X₁ is OH, NH₂, NHR^{A1}, NR^{A1}R^{A2}, and OR^{A1}; Y₁ is CH₂N(R^{B})(CH₂CH₂O)_{b}CH₂CH₂Z₂; each of R^{A1} and R^{A2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{B} is H or C₁₋₄ alkyl; b is an integer from 1 to 10; Z₂ is NH₂, NHR^{C1}, NR^{C1}R^{C2}, or NR^{C1}R^{C2}R^{C3}; and each of R^{C1}, R^{C2}, and R^{C3} is, independently, selected from C₁₋₄ alkyl, C₂₋₄ alkenyl, and C₂₋₄ alkynyl, or a salt or prodrug thereof.

7. The compound of claim 5, wherein T is —NH(CH₂)₉CH₃, X₁ is OH, NH₂, NHR^{A1}, NR^{A1}R^{A2}, and OR^{A1}; Y₁ is CH₂N(R^{B})(CH₂CH₂O)_{b}CH₂CH₂Z₂; each of R^{A1} and R^{A2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{B} is H or C₁₋₄ alkyl; b is an integer from 1 to 10; Z₂ is NH₂, NHR^{C1}, NR^{C1}R^{C2}, or NR^{C1}R^{C2}R^{C3}; and each of R^{C1}, R^{C2}, and R^{C3} is, independently, selected from C₁₋₄ alkyl, C₂₋₄ alkenyl, and C₂₋₄ alkynyl, or a salt or prodrug thereof.

8. The compound of claim 5, wherein T is —NHCH₂CH₂NH(CH₂)₉CH₃, X₁ is OH, NH₂, NHR^{A1}, NR^{A1}R^{A2}, and OR^{A1}; Y₁ is CH₂N(R^{B})(CH₂CH₂O)_{b}CH₂CH₂Z₂; each of R^{A1} and R^{A2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{B} is H or C₁₋₄ alkyl; b is an integer from 1 to 10; Z₂ is NH₂, NHR^{C1}; NR^{C1}R^{C2}; or NR^{C1}R^{C2}R^{C3}; and each of R^{C1}, R^{C2}, and R^{C3} is, independently, selected from C₁₋₄ alkyl, C₂₋₄ alkenyl, and C₂₋₄ alkynyl, or a salt or prodrug thereof.

9. The compound of claim 5, wherein T is p-(p-chlorophenyl)benzyl-NH—, X₁ is OH, NH₂, NHR^{A1}, NR^{A1}R^{A2}, and OR^{A1}; Y¹ is CH₂N(R^{B})(CH₂CH₂O)_{b}CH₂CH₂Z₂; each of R^{A1} and R^{A2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{B} is H or C₁₋₄ alkyl; b is an integer from 1 to 10; Z₂ is NH₂, NHR^{C1}; NR^{C1}R^{C2}; or NR^{C1}R^{C2}R^{C3}; and each of R^{C1}, R^{C2}, and R^{C3} is, independently, selected from C₁₋₄ alkyl, C₂₋₄ alkenyl, and C₂₋₄ alkynyl, or a salt or prodrug thereof.

10. The compound of claim 5, wherein T is 4-phenylbenzyl-NH—, X₁ is OH, NH₂, NHR^{A1}, NR^{A1}R^{A2}, and OR^{A1}; Y₁ is CH₂N(R^{B})(CH₂CH₂O)_{b}CH₂CH₂Z₂; each of R^{A1} and R^{A2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{B} is H or C₁₋₄ alkyl; b is an integer from 1 to 10; Z₂ is NH₂, NHR^{C1}; NR^{C1}R^{C2}; or NR^{C1}R^{C2}R^{C3}; and each of R^{C1}, R^{C2}, and R^{C3} is, independently, selected from C₁₋₄ alkyl, C₂₋₄ alkenyl, and C₂₋₄ alkynyl, or a salt or prodrug thereof.

11. The compound of claim 5, wherein T is 4-[(3,4-dichlorophenyl)methoxy]benzyl-NH—, X₁ is OH, NH₂, NHR^{A1}, NR^{A1}R^{A2}, and OR^{A1}; Y₁ is CH₂N(R^{B})(CH₂CH₂O)_{b}CH₂CH₂Z₂; each of R^{A1} and R^{A2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{B} is H or C₁₋₄ alkyl; b is an integer from 1 to 10; Z₂ is NH₂, NHR^{C1}, NR^{C1}R^{C2}, or NR^{C1}R^{C2}R^{C3}; and each of R^{C1}, R^{C2}, and R^{C3} is, independently, selected from C₁₋₄ alkyl, C₂₋₄ alkenyl, and C₂₋₄ alkynyl, or a salt or prodrug thereof.

12. The compound of claim 5, wherein T is —NH₂, X₁ is N(R^{A})(CH₂CH₂O)ₐCH₂CH₂Z₁; Y₁ is selected from H, CH₂NH₂, CH₂NHCOR^{B1}, CH₂NHCONHR^{B1}, CH₂NHCONR^{B1}R^{B2}, CH₂NHC(O)OR^{B1}, CH₂NHR^{B1}, CH₂NR^{B1}R^{B2}; CH₂NHSO₂R^{B1}, CH₂NHSO₂NHR^{B1}, CH₂NHSO₂NR^{B1}R^{B2}, and CH₂NHCH₂PO(OH)₂; each of R^{B1} and R^{B2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{A} is H or C₁₋₄ alkyl; a is an integer from 1 to 10; Z₂ is NH₂, NHR^{C1}, NR^{C1}R^{C2}, or NR^{C1}R^{C2}R^{C3}; and each of R^{C1}, R^{C2} and R^{C3} is, independently, selected from C₁₋₄ alkyl, C₂₋₄ alkenyl, and C₂₋₄ alkynyl, or a salt or prodrug thereof.

13. The compound of claim 5, wherein T is —NH(CH₂)₉CH₃, X₁ is N(R^{A})(CH₂CH₂O)ₐCH₂CH₂Z₁; Y₁ is selected from H, CH₂NH₂, CH₂NHCOR^{B1}, CH₂NHCONHR^{B1}, CH₂NHCONR^{B1}R^{B2}, CH₂NHC(O)OR^{B1}, CH₂NHR^{B1}, CH₂NR^{B1}R^{B2}, CH₂NHSO₂R^{B1}, CH₂NHSO₂NHR^{B1}, CH₂NHSO₂NR^{B1}R^{B2}, and CH₂NHCH₂PO(OH)₂; each of R^{B1} and R^{B2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{A} is H or C₁₋₄ alkyl; a is an integer from 1 to 10; Z₂ is NH₂, NHR^{C1}, NR^{C1}R^{C2}, or NR^{C1}R^{C2}R^{C3}; and each of R^{C1}, R^{C2} and R^{C3} is, independently, selected from C₁₋₄ alkyl, C₂₋₄ alkenyl, and C₂₋₄ alkynyl, or a salt or prodrug thereof.

14. The compound of claim 5, wherein T is —NHCH₂CH₂NH(CH₂)₉CH₃, X₁ is N(R^{A})(CH₂CH₂O)ₐCH₂CH₂Z₁; Y₁ is selected from H, CH₂NH₂, CH₂NHCOR^{B1}, CH₂NHCONHR^{B1}, CH₂NHCONR^{B1}R^{B2}, CH₂NHC(O)OR^{B1}, CH₂NHR^{B1}, CH₂NR^{B1}R^{B2}; CH₂NHSO₂R^{B1}, CH₂NHSO₂NHR^{B1}, CH₂NHSO₂NR^{B1}R^{B2}, and CH₂NHCH₂PO(OH)₂; each of R^{B1} and R^{B2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{A} is H or C₁₋₄ alkyl; a is an integer from 1 to 10; Z₂ is NH₂, NHR^{C1}, NR^{C1}R^{C2}, or NR^{C1}R^{C2}R^{C3}; and each of R^{C1}, R^{C2} and R^{C3} is, independently, selected from C₁₋₄ alkyl, C₂₋₄ alkenyl, and C₂₋₄ alkynyl, or a salt or prodrug thereof.

15. The compound of claim 5, wherein T is p-(p-chlorophenyl)benzyl-NH—, X₁ is N(R^{A})(CH₂CH₂O)ₐCH₂CH₂Z₁; Y₁ is selected from H, CH₂NH₂, CH₂NHCOR^{B1}, CH₂NHCONHR^{B1}, CH₂NHCONR^{B1}R^{B2}, CH₂NHC(O)OR^{B1}, CH₂NHR^{B1}, CH₂NR^{B1}R^{B2}; CH₂NHSO₂R^{B1}, CH₂NHSO₂NHR^{B1}, CH₂NHSO₂NR^{B1}R^{B2}, and CH₂NHCH₂PO(OH)₂; each of R^{B1} and R^{B2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{A} is H or C₁₋₄ alkyl; a is an integer from 1 to 10; Z₂ is NH₂, NHR^{C1}, NR^{C1}R^{C2}, or NR^{C1}R^{C2}R^{C3}; and each of R^{C1}, R^{C2} and R^{C3} is, independently, selected from C₁₋₄ alkyl, C₂₋₄ alkenyl, and C₂₋₄ alkynyl, or a salt or prodrug thereof.

16. The compound of claim 5, wherein T is 4-phenylbenzyl-NH—, X₁ is N(R^{A})(CH₂CH₂O)ₐCH₂CH₂Z₁; Y₁ is selected from H, CH₂NH₂, CH₂NHCOR^{B1}, CH₂NHCONHR^{B1}, CH₂NHCONR^{B1}R^{B2}, CH₂NHC(O)OR^{B1}, CH₂NHR^{B1}, CH₂NR^{B1}R^{B2}; CH₂NHSO₂R^{B1}, CH₂NHSO₂NHR^{B1}, CH₂NHSO₂NR^{B1}R^{B2}, and CH₂NHCH₂PO(OH)₂; each of R^{B1} and R^{B2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{A} is H or C₁₋₄ alkyl; a is an integer from 1 to 10; Z₂ is NH₂, NHR^{C1}, NR^{C1}R^{C2}, or NR^{C1}R^{C2}R^{C3}; and each of R^{C1}, R^{C2} and R^{C3} is, independently, selected from C₁₋₄ alkyl, C₂₋₄ alkenyl, and C₂₋₄ alkynyl, or a salt or prodrug thereof.

17. The compound of claim 5, wherein T is 4-[(3,4-dichlorophenyl)methoxy]benzyl-NH—, X₁ is N(R^{A})(CH₂CH₂O)ₐCH₂CH₂Z₁; Y₁ is selected from H, CH₂NH₂, CH₂NHCOR^{B1}, CH₂NHCONHR^{B1}, CH₂NHCONR^{B1}R^{B2}, CH₂NHC(O)OR^{B1}, CH₂NHR^{B1}, CH₂NR^{B1}R^{B2}; CH₂NHSO₂R^{B1}, CH₂NHSO₂NHR^{B1}, CH₂NHSO₂NR^{B1}R^{B2}, and CH₂NHCH₂PO(OH)₂; each of R^{B1} and R^{B2} is, independently, selected from C₁₋₁₂ alkyl, C₂₋₁₂ alkenyl, C₂₋₁₂ alkynyl, C₆₋₁₂ aryl, C₇₋₁₆ alkaryl, C₃₋₁₀ alkheterocyclyl, and C₁₋₁₂ heteroalkyl; R^{A} is H or C₁₋₄ alkyl; a is an integer from 1 to 10; Z₂ is NH₂, NHR^{C1}, NR^{C1}R^{C2}, or NR^{C1}R^{C2}R^{C3}; and each of R^{C1}, R^{C2} and R^{C3} is, independently, selected from C₁₋₄ alkyl, C₂₋₄ alkenyl, and C₂₋₄ alkynyl, or a salt or prodrug thereof.

18. The compound of claim 5, wherein T is —NH₂, X₁ is N(R^{A})(CH₂CH₂O)ₐCH₂CH₂Z₁, Y₁ is CH₂N(R^{B})(CH₂CH₂O)_{b}CH₂CH₂Z₂, each of R^{A} and R^{B} is, independently, selected from H and C₁₋₄ alkyl, a is an integer from 1 to 10, b is an integer from 1 to 10, each of Z₁ and Z₂ is, independently, selected from NH₂, NHR^{C1}, NR^{C1}R^{C2}, and NR^{C1}R^{C2}R^{C3}, and each of R^{C1}, R^{C2}, and R^{C3} is, independently, selected from C₁₋₄ alkyl, C₂₋₄ alkenyl, and C₂₋₄ alkynyl, or a salt or prodrug thereof.

19. The compound of claim 5, wherein T is —NH(CH₂)₉CH₃, X₁ is N(R^{A})(CH₂CH₂O)ₐCH₂CH₂Z₁, Y₁ is CH₂N(R^{B})(CH₂CH₂O)_{b}CH₂CH₂Z₂, each of R^{A} and R^{B} is, independently, selected from H and C₁₋₄ alkyl, a is an integer from 1 to 10, b is an integer from 1 to 10, each of Z₁ and Z₂ is, independently, selected from NH₂, NHR^{C1}, NR^{C1}R^{C2}, and NR^{C1}R^{C2}R^{C3}, and each of R^{C1}, R^{C2}, and R_{C3} is, independently, selected from C₁₋₄ alkyl, C₂₋₄ alkenyl, and C₂₋₄ alkynyl, or a salt or prodrug thereof.

20. The compound of claim 5, wherein T is —NHCH₂CH₂NH(CH₂)₉CH₃, X₁ is N(R^{A})(CH₂CH₂O)ₐCH₂CH₂Z₁, Y₁ is CH₂N(R^{B})(CH₂CH₂O)_{b}CH₂CH₂Z₂, each of R^{A} and R^{B} is, independently, selected from H and C₁₋₄ alkyl, a is an integer from 1 to 10, b is an integer from 1 to 10, each of Z₁ and Z₂ is, independently, selected from NH₂, NHR^{C1}, NR^{C1}R^{C2}, and NR^{C1}R^{C2}R^{C3}, and each of R^{C1}, R^{C2}, and R^{C3} is, independently, selected from C₁₋₄ alkyl, C₂₋₄ alkenyl, and C₂₋₄ alkynyl, or a salt or prodrug thereof.

21. The compound of claim 5, wherein T is p-(p-chlorophenyl)benzyl-NH—, X₁ is N(R^{A})(CH₂CH₂O)ₐCH₂CH₂Z₁, Y₁ is CH₂N(R^{B})(CH₂CH₂O)_{b}CH₂CH₂Z₂, each of R^{A} and R^{B} is, independently, selected from H and C₁₋₄ alkyl, a is an integer from 1 to 10, b is an integer from 1 to 10, each of Z₁ and Z₂ is, independently, selected from NH₂, NHR^{C1}, NR^{C1}R^{C2}, and NR^{C1}R^{C}2R^{C3}, and each of R^{C1}, R^{C2}, and R^{C3} is, independently, selected from C₁₋₄ alkyl, C₂₋₄ alkenyl, and C₂₋₄ alkynyl, or a salt or prodrug thereof.

22. The compound of claim 5, wherein T is 4-phenylbenzyl-NH—, X₁ is N(R^{A})(CH₂CH₂O)ₐCH₂CH₂Z₁, Y₁ is CH₂N(R^{B})(CH₂CH₂O)_{b}CH₂CH₂Z₂, each of R^{A} and R^{B} is, independently, selected from H and C₁₋₄ alkyl, a is an integer from 1 to 10, b is an integer from 1 to 10, each of Z₁ and Z₂ is, independently, selected from NH₂, NHR^{C1}, NR^{C1}R^{C2}, and NR^{C1}R^{C2}R^{C3}, and each of R^{C1}, R^{C2}, and R^{C3} is, independently, selected from C₁₋₄ alkyl, C₂₋₄ alkenyl, and C₂₋₄ alkynyl, or a salt or prodrug thereof.

23. The compound of claim 5, wherein T is 4-[(3,4-dichlorophenyl)methoxy]benzyl-NH—, X₁ is N(R^{A})(CH₂CH₂O)ₐCH₂CH₂Z₁, Y₁ is CH₂N(R^{B})(CH₂CH₂O)_{b}CH₂CH₂Z₂, each of R^{A} and R^{B} is, independently, selected from H and C₁₋₄ alkyl, a is an integer from 1 to 10, b is an integer from 1 to 10, each of Z₁ and Z₂ is, independently, selected from NH₂, NHR^{C1}, NR^{C1}R^{C2}, and NR^{C1}R^{C2}R^{C3}, and each of R^{C1}, R^{C2}, and R^{C3} is, independently, selected from C₁₋₄ alkyl, C₂₋₄ alkenyl, and C₂₋₄ alkynyl, or a salt or prodrug thereof.

24. The compound of claim 1, wherein said compound is further described by formula (VI), or a salt or prodrug thereof: 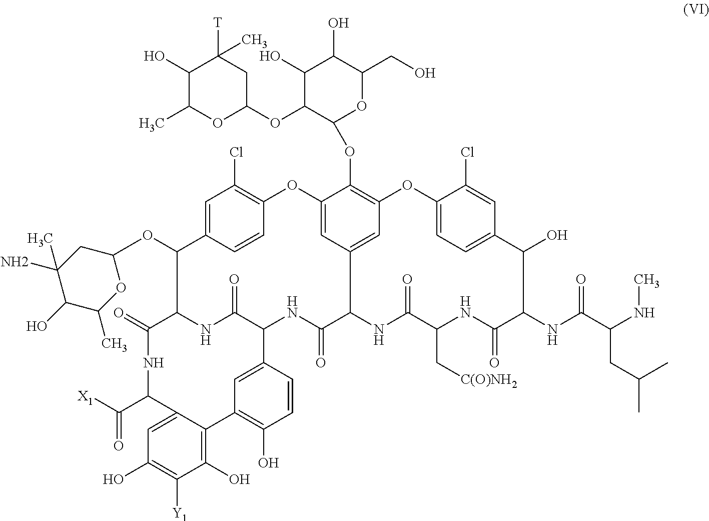
wherein X₁, Y₁, and T are as defined in formula (I).

25. The compound of claim 24, wherein T is selected from —NH₂, —NH(CH₂)₉CH₃, —NHCH₂CH₂NH(CH₂)₉CH₃, p-(p-chlorophenyl)benzyl-NH—, 4-phenylbenzyl-NH—, and 4-[(3,4-dichlorophenyl)methoxy]benzyl-NH—.

26. The compound of claim 1, wherein said compound is further described by formula (VII), or a salt or prodrug thereof: 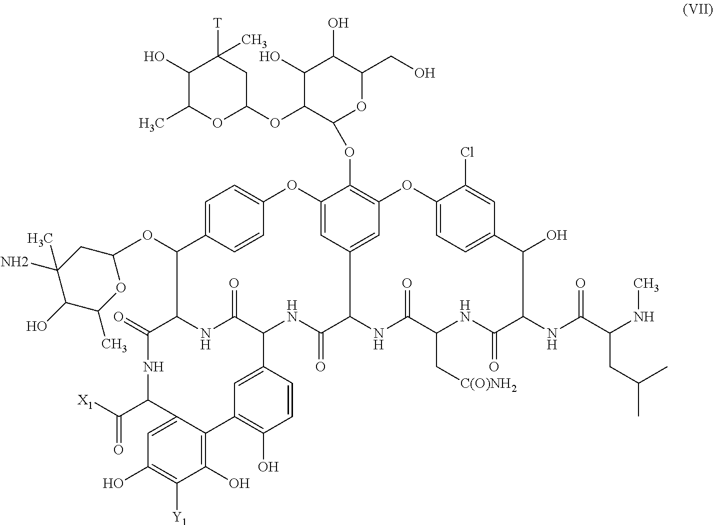
wherein X₁, Y₁, and T are as defined in formula (I).

27. The compound of claim 26, wherein T is selected from —NH₂, —NH(CH₂)₉CH₃, —NHCH₂CH₂NH(CH₂)₉CH₃, p-(p-chlorophenyl)benzyl-NH—, 4-phenylbenzyl-NH—, and 4-[(3,4-dichlorophenyl)methoxy]benzyl-NH—.

28. The compound of claim 1, wherein said compound is further described by formula (VIII), or a salt or prodrug thereof: 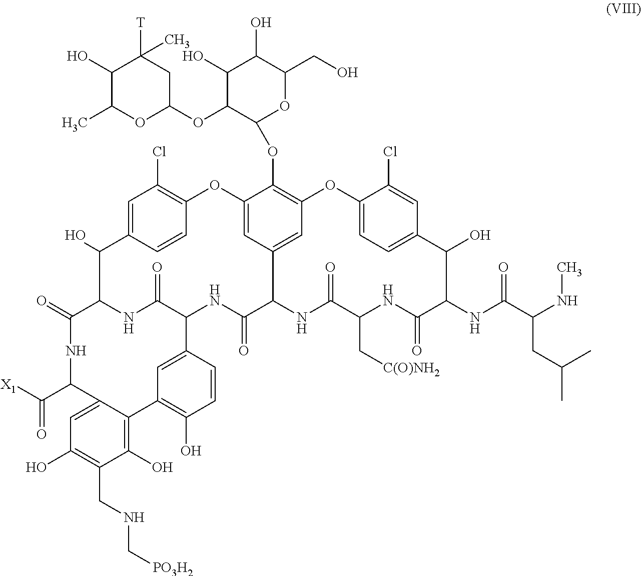
wherein X₁ and T are as defined in formula (I).

29. The compound of claim 28, wherein T is selected from —NH₂, —NH(CH₂)₉CH₃, —NHCH₂CH₂NH(CH₂)₉CH₃, p-(p-chlorophenyl)benzyl-NH—, 4-phenylbenzyl-NH—, and 4-[(3,4-dichlorophenyl)methoxy]benzyl-NH—.

30. A pharmaceutical composition comprising a compound of claim 1, or a salt or prodrug thereof, and a pharmaceutically acceptable excipient.

31. A method of treating a bacterial infection in a subject, said method comprising administering to said subject a compound of claim 1, or a salt or prodrug thereof, in an amount sufficient to treat said infection.

32. The method of claim 31, wherein said infection is selected from community-acquired pneumonia, upper and lower respiratory tract infection, skin and soft tissue infection, bone and joint infection, hospital-acquired lung infection, acute bacterial otitis media, bacterial pneumonia, complicated infection, noncomplicated infection, pyelonephritis, intra-abdominal infection, deep-seated abcess, bacterial sepsis, central nervous system infection, bacteremia, wound infection, peritonitis, meningitis, infections after burn, urogenital tract infection, gastro-intestinal tract infection, pelvic inflammatory disease, endocarditis, intravascular infection, complicated skin and skin structure infection, complicated intra-abdominal infection, hospital acquired pneumonia, ventilator associated pneumonia, pseudomembranous colitis, enterocolitis, and infections associated with prosthetics or dialysis; or said compound is administered for prophylaxis against an infection associated with a surgical procedure or implantation of a prosthetic device.

33. The method of claim 31, wherein said compound is administered orally, or intravenously.

34. A method of killing a bacterial cell, said method comprising contacting said cell with a compound claim 1, or a salt or prodrug thereof, in an amount sufficient to kill said bacterial cell.

35. The method of claim 34, wherein said bacterial cell is selected from Staphylococcus spp; Streptococcus spp; Enterococcus spp; Clostridium spp; Bacillus spp; Staphylococcus aureus, including methicillin-susceptible (MSSA), methicillin-resistant (MRSA), vancomycin-intermediate (VISA), heterogeneous VISA (hVISA), and vancomycin-resistant (VRSA) strains; Staphylococcus epidermidis, including methicillin susceptible and resistant strains; Enterococcus faecium, including VanA-type (VRE) and VanB-type (VRE) resistant strains; Enterococcus faecalis, including VanA-type (VRE) and VanB-type (VRE) resistant strains; Enterococcus casseliflavus and Enterococcus gallinarum, including vanC-carrying strains; Streptococcus pneumoniae, including multi-drug resistant strains; Streptococcus pyogenes and Streptococcus agalactiae, including β-hemolytic strains; and Bacillus anthracis.

36. A pharmaceutical composition in oral dosage form comprising a vancomycin class compound, or a salt or prodrug thereof, and an additive selected from sugar esters, alkyl saccharides, acyl carnitines, glycerides, chitosan and derivatives thereof, amido fatty acids, fatty acids and salts or esters thereof, polyethylene glycol alkyl ethers, poly-D-lysine, N-acetyl-L-cystine, and combinations thereof, wherein said additive is present in an amount sufficient to increase the oral bioavailability of said vancomycin class compound.

37-53. (canceled)

54. A method of synthesizing the acid addition salt of a compound of formula (X): 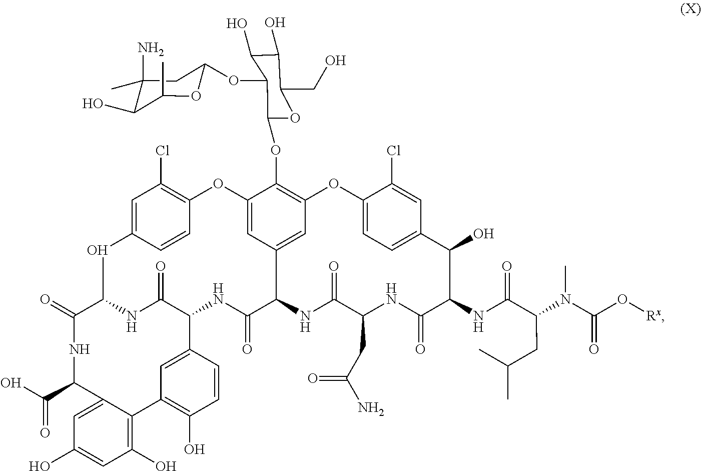
said method comprising reacting the mono acid addition salt of vancomycin with a dicarbonate in an organic solvent to form an acid addition salt of a compound of formula (X), said dicarbonate having the formula R^{X}—OC(O)—O—C(O)O—R^{X}, wherein R^{X} is selected from C₁₋₁₂ alkyl, C₂₋₁₂ alkenyl, C₂₋₁₂ alkynyl, and C₇₋₁₆ alkaryl, and
wherein the ratio of acid to vancomycin is from about 0.85:1 to 1.15:1.

55. The method of claim 54, further comprising (i) dissolving vancomycin, or an acid addition salt thereof, in an organic solvent and (ii) adjusting the pH of the solution with base or acid to produce a ratio of acid to vancomycin of from about 0.95:1 to 1.05:1 prior to reaction with said dicarbonate.

56. The method of claim 54, wherein said acid addition salt of vancomycin is selected from vancomycin hydrochloride, vancomycin hydrobromide, vancomycin hydroiodide, vancomycin sulfate, vancomycin phosphate and vancomycin methansulfonate.

57. The method of any of claim 54, wherein said dicarbonate is selected from di-tert-butyl dicarbonate, dibenzyl dicarbonate, and diallyl dicarbonate.

58. A method of synthesizing a vancomycin class compound, said method comprising (i) performing the method of claim 54 to produce a carbamate-protected vancomycin of formula (X), or a salt thereof, (ii) alkylating the amine bearing saccharide group of said carbamate-protected vancomycin, coupling an amine to the C-terminal carboxylate of said carbamate-protected vancomycin, and/or adding an aminomethyl substituent the resorcinol ring of said carbamate-protected vancomycin via a Mannich reaction, and (iii) removing the carbamate protecting group to produce a vancomycin class compound having antibacterial activity.

59. The method of claim 58, wherein said vancomycin class compound is telavancin.

60. The method of claim 58, wherein said vancomycin class compound is a compound of claim 1.
